(19) United States
(12) Patent Application Publication
(10) Pub. No.: US 2008/0275029 A1
(43) Pub. Date: Nov. 6, 2008

(54) Compounds for Treating Protein-Kinase Mediated Disorders

(75) Inventors: Valerio Berdini, Cambridge (GB); Robert George Boyle, Cambridge (GB); Gordon Saxty, Cambridge (GB); Marinus Leendert Verdonk, Burwell (GB); Steven John Woodhead, Cambridge (GB); Paul Graham Wyatt, Perth (GB); Hannah Fiona Sore, Cambridge (GB); David Winter Walker, Cambridge (GB); John Caldwell, Sutton (GB); Ian Collins, Redhill (GB)

Correspondence Address: HESLIN ROTHENBERG FARLEY & MESITI PC, 5 COLUMBIA CIRCLE, ALBANY, NY, 12203, US

(73) Assignees: ASTEX THERAPEUTICS LIMITED, Cambridge (GB); CANCER RESEARCH TECHNOLOGY LIMITED, London (GB); THE INSTITUTE OF CANCER RESEARCH: ROYAL CANCER HOS, London (GB)

(21) Appl. No.: 11/718,943

(22) PCT Filed: Nov. 9, 2005
(86) PCT No.: PCT/GB2005/004323

§ 371 Date: Jun. 10, 2008

Related U.S. Application Data
(60) Provisional application No. 60/626,403, filed on Nov. 9, 2004.

(30) Foreign Application Priority Data
Nov. 9, 2004 (GB) 0424742.5

(51) Int. Cl. A61K 31/517; C07D 239/88; C07D 401/12; C07D 401/04; A61K 31/496; A61K 31/551; A61P 35/00; C12N 5/06; A61K 31/5377; C07D 401/06; C07D 403/12; C07D 413/14

(52) U.S. Cl. 514/218; 514/266.3; 544/283; 514/266.22; 514/266.2; 514/252.17; 540/575; 514/234.5; 544/116; 435/375

(57) ABSTRACT

The invention provides a compound of the formula (I) or a salt, solvate, tautomer or N-oxide thereof for use in the treatment or prophylaxis of a disease state or condition mediated by protein kinase A and/or protein kinase B;

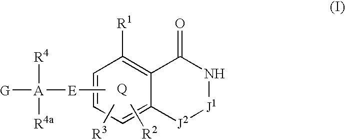

wherein the ring Q is a benzene ring; J²-J¹ is N═CR⁷ or R^{1a}N—CO; G is OH or NR⁵R⁶; E is CONR⁷, NR⁷CO, C(R⁸)═C(R⁸) or (X)ₘ(CR⁸R^{8a})ₙ where X is O, S or NR⁷; provided that when J²-J¹ is R^{1a}N—CO, E is other than NR⁷CO; m and n are each 0 or 1, where m+n=1 or 2; A is a bond and R⁴ and R^{4a} are absent or A is a saturated optionally substituted C₁₋₇ hydrocarbon linker group having a maximum chain length of 5 atoms extending between E and G, one carbon atom in the linker group A being optionally replaced by O or N; R¹, R^{1a}, R², and R³ are each H; halogen; C₁₋₆ hydrocarbyl optionally substituted by halogen, OH or C₁₋₂ alkoxy; CN; CONHR⁸; NH₂; NHCOR¹⁰ or NHCONHR¹⁰; R⁴ is H or C₁₋₄ alkyl; R^{4a} is H, C₁₋₄ alkyl or a group R⁹; R⁵ and R⁶ are each selected from H, R⁹ and C₁₋₄ hydrocarbyl optionally substituted by halogen, C₁₋₂ alkoxy or R⁹; or NR⁵R⁶ forms a saturated 4-7 membered monocyclic heterocyclic group; R⁷ is H or C₁₋₄ alkyl; R⁸ and R^{8a} each H or saturated C₁₋₄ hydrocarbyl optionally substituted by fluorine; R⁹ is a monocyclic or bicyclic carbocyclic or heterocyclic group containing up to 3 ring heteroatoms selected from N, O and S; or R⁴, R^{4a} and A together form a saturated monocyclic 4-7 membered heterocycle; or NR⁵R⁶, R⁴ and A form a saturated 4-7 membered monocyclic heterocycle; or R⁴, together with R⁷ or R⁸ and A and E form a 4-7 membered saturated monocyclic heterocycle; or NR⁵R⁶ and R⁷ or R⁸ together with A and E form a 4-7 membered saturated monocyclic heterocycle; and R¹⁰ is optionally substituted phenyl or benzyl.

### RELATED APPLICATIONS

This application is related to U.S. provisional patent application U.S. 60/626,403 (filed 9 Nov. 2004), the contents of which are incorporated herein by reference.

### TECHNICAL FIELD

This invention relates to quinazolinone compounds that inhibit or modulate the activity of protein kinase A (PKA) and protein kinase B (PKB), to the use of the compounds in the treatment or prophylaxis of disease states or conditions mediated by PKA and PKB, and to novel compounds having PKA and PKB inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J, 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

Apoptosis or programmed cell death is an important physiological process which removes cells no longer required by an organism. The process is important in early embryonic growth and development allowing the non-necrotic controlled breakdown, removal and recovery of cellular components. The removal of cells by apoptosis is also important in the maintenance of chromosomal and genomic integrity of growing cell populations. There are several known checkpoints in the cell growth cycle at which DNA damage and genomic integrity are carefully monitored. The response to the detection of anomalies at such checkpoints is to arrest the growth of such cells and initiate repair processes. If the damage or anomalies cannot be repaired then apoptosis is initiated by the damaged cell in order to prevent the propagation of faults and errors. Cancerous cells consistently contain numerous mutations, errors or rearrangements in their chromosomal DNA.

It is widely believed that this occurs in part because the majority of tumours have a defect in one or more of the processes responsible for initiation of the apoptotic process. Normal control mechanisms cannot kill the cancerous cells and the chromosomal or DNA coding errors continue to be propagated. As a consequence restoring these pro-apoptotic signals or suppressing unregulated survival signals is an attractive means of treating cancer.

The signal transduction pathway containing the enzymes phosphatidylinositol 3-kinase (PI3K), PDK1 and PKB amongst others, has long been known to mediate increased resistance to apoptosis or survival responses in many cells. There is a substantial amount of data to indicate that this pathway is an important survival pathway used by many growth factors to suppress apoptosis. The enzymes of the PI3K family are activated by a range of growth and survival factors e.g. EGF, PDGF and through the generation of polyphosphatidylinositols, initiates the activation of the downstream signalling events including the activity of the kinases PDK1 and protein kinase B (PKB) also known as akt. This is also true in host tissues, e.g. vascular endothelial cells as well as neoplasias. PKB is a protein ser/thr kinase consisting of a kinase domain together with an N-terminal PH domain and C-terminal regulatory domain. The enzyme PKBₐₗₚₕₐ (akt1) itself is phosphorylated on Thr 308 by PDK1 and on Ser 473 by a kinase referred to as PDK2, whereas PKB_{beta} (akt2) is phosphorylated on Thr 309 and on Ser 474, and PKB_{gamma} (akt3) is phosphorylated on Thr 305 and on Ser 472.

At least 10 kinases have been suggested to function as a Ser 473 kinase including mitogen-activated protein (MAP) kinase-activated protein kinase-2 (MK2), integrin-linked kinase (ILK), p38 MAP kinase, protein kinase Calpha (PKCalpha), PKCbeta, the NIMA-related kinase-6 (NEK6), the mammalian target of rapamycin (mTOR), the double-stranded DNA-dependent protein kinase (DNK-PK), and the ataxia telangiectasia mutated (ATM) gene product. Available data suggest that multiple systems may be used in cells to regulate the activation of PKB. Full activation of PKB requires phosphorylation at both sites whilst association between PIP3 and the PH domain is required for anchoring of the enzyme to the cytoplasmic face of the lipid membrane providing optimal access to substrates.

Activated PKB phosphorylates a range of substrates contributing to the overall survival response. Whilst we cannot be certain that we understand all of the factors responsible for mediating the PKB dependent survival response, some important actions are believed to be phosphorylation and inactivation of the pro-apoptotic factor BAD and caspase 9, phosphorylation of Forkhead transcription factors e.g. FKHR leading to their exclusion from the nucleus, and activation of the NfkappaB pathway by phosphorylation of upstream kinases in the cascade, as well as the phosphorylation of ASK-1 (apoptosis signal regulating kinase 1) thereby deactivating it and hence preventing the transmission of apoptotic signals.

In addition to the anti-apoptotic and pro-survival actions of the PKB pathway, the enzyme also plays an important role in promoting cell proliferation. This action is again likely to be mediated via several actions, some of which are thought to be phosphorylation and inactivation of the cyclin dependent kinase inhibitor of p21^{CiP1/WAF1}, and phosphorylation and activation of mTOR, a kinase controlling several aspects of cell size, growth and protein translation.

The phosphatase PTEN which dephosphorylates and inactivates polyphosphatidylinositols is a key tumour suppressor protein which normally acts to regulate the PI3K/PKB survival pathway. The significance of the PI3K/PKB pathway in tumourigenesis can be judged from the observation that PTEN is one of the most common targets of mutation in human tumours, with mutations in this phosphatase having been found in ˜50% or more of melanomas (Guldberg et al 1997, Cancer Research 57, 3660-3663) and advanced prostate cancers (Cairns et al 1997 Cancer Research 57, 4997). These observations and others suggest that a wide range of tumour types are dependent on the enhanced PKB activity for growth and survival and would respond therapeutically to appropriate inhibitors of PKB.

There are 3 closely related isoforms of PKB called alpha, beta and gamma, which genetic studies suggest have distinct but overlapping functions. Evidence suggests that they can all independently play a role in cancer. For example PKB beta has been found to be over-expressed or activated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330), PKB alpha is amplified in human gastric, prostate and breast cancer (Staal 1987, PNAS 84, 5034-5037; Sun et al 2001, Am. J. Pathol. 159, 431-437) and increased PKB gamma activity has been observed in steroid independent breast and prostate cell lines (Nakatani et al 1999, J. Biol. Chem. 274, 21528-21532).

The PKB pathway also functions in the growth and survival of normal tissues and may be regulated during normal physiology to control cell and tissue function.

Thus disorders associated with undesirable proliferation and survival of normal cells and tissues may also benefit therapeutically from treatment with a PKB inhibitor. Examples of such disorders are disorders of immune cells associated with prolonged expansion and survival of cell population leading to a prolonged or up regulated immune response. For example, T and B lymphocyte response to cognate antigens or growth factors such as interferon gamma activates the PI3K/PKB pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PKB pathway contributes an important survival signal preventing the normal mechanisms by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding inappropriately to foreign antigens is a feature of another set of conditions such as allergic responses and asthma. In summary inhibition of PKB could provide a beneficial treatment for immune disorders.

Other examples of inappropriate expansion, growth, proliferation, hyperplasia and survival of normal cells in which PKB may play a role include but are not limited to atherosclerosis, cardiac myopathy and glomerulonephritis.

In addition to the role in cell growth and survival, the PKB pathway functions in the control of glucose metabolism by insulin. Available evidence from mice deficient in the alpha and beta isoforms of PKB suggests that this action is mediated by the beta isoform primarily. As a consequence, modulators of PKB activity may also find utility in diseases in which there is a dysfunction of glucose metabolism and energy storage such as diabetes, metabolic disease and obesity.

Cyclic AMP-dependent protein kinase (PKA) is a serine/threonine protein kinase that phosphorylates a wide, range of substrates and is involved in the regulation of many cellular processes including cell growth, cell differentiation, ion-channel conductivity, gene transcription and synaptic release of neurotransmitters. In its inactive form, the PKA holoenzyme is a tetramer comprising two regulatory subunits and two catalytic subunits.

PKA acts as a link between G-protein mediated signal transduction events and the cellular processes that they regulate. Binding of a hormone ligand such as glucagon to a transmembrane receptor activates a receptor-coupled G-protein (GTP-binding and hydrolyzing protein). Upon activation, the alpha subunit of the G protein dissociates and binds to and activates adenylate cyclase, which in turn converts ATP to cyclic-AMP (cAMP). The cAMP thus produced then binds to the regulatory subunits of PKA leading to dissociation of the associated catalytic subunits. The catalytic subunits of PKA, which are inactive when associated with the regulatory sub-units, become active upon dissociation and take part in the phosphorylation of other regulatory proteins.

For example, the catalytic sub-unit of PKA phosphorylates the kinase Phosphorylase Kinase which is involved in the phosphorylation of Phosphorylase, the enzyme responsible for breaking down glycogen to release glucose. PKA is also involved in the regulation of glucose levels by phosphorylating and deactivating glycogen synthase. Thus, modulators of PKA activity (which modulators may increase or decrease PKA activity) may be useful in the treatment or management of diseases in which there is a dysfunction of glucose metabolism and energy storage such as diabetes, metabolic disease and obesity.

PKA has also been established as an acute inhibitor of T cell activation. Anndahl et al, have investigated the possible role of PKA type I in HIV-induced T cell dysfunction on the basis that T cells from HIV-infected patients have increased levels of cAMP and are more sensitive to inhibition by cAMP analogues than are normal T cells. From their studies, they concluded that increased activation of PKA type I may contribute to progressive T cell dysfunction in HIV infection and that PKA type I may therefore be a potential target for immunomodulating therapy.—Aandahl, E. M., Aukrust, P., Skålhegg, B. S., Müller, F., Frøland, S. S., Hansson, V., Tásken, K. Protein kinase A type I antagonist restores immune responses of T cells from HIV-infected patients. FASEB J. 12, 855-862 (1998).

It has also been recognised that mutations in the regulatory sub-unit of PKA can lead to hyperactivation in endocrine tissue.

Because of the diversity and importance of PKA as a messenger in cell regulation, abnormal responses of cAMP can lead to a variety of human diseases such as irregular cell growth and proliferation (Stratakis, C. A.; Cho-Chung, Y. S.; Protein Kinase A and human diseases. Trends Endrocri. Metab. 2002, 13, 50-52). Over-expression of PKA has been observed in a variety of human cancer cells including those from ovarian, breast and colon patients. Inhibition of PKA would therefore be an approach to treatment of cancer (Li, Q.; Zhu, G-D.; Current Topics in Medicinal Chemistry, 2002, 2, 939-971).

For a review of the role of PKA in human disease, see for example, Protein Kinase A and Human Disease, Edited by Constantine A. Stratakis, Annals of the New York Academy of Sciences, Volume 968, 2002, ISBN 1-57331-412-9.

### PRIOR ART

Several classes of compounds have been disclosed as having PKA and PKB inhibitory activity.

For example, a class of isoquinolinyl-sulphonamido-diamines having PKB inhibitory activity is disclosed in WO 01/91754 (Yissum).

WO 93/13072 (Italfarmaco) discloses a class of bis-sulphonamido diamines as protein kinase inhibitors.

WO 2005/061463 (Astex Technology et al.), which was published after the earliest priority date of the present application, discloses pyrazole derivatives as inhibitors of PKA and PKB.

US2003/0220355 (Warner-Lambert) discloses a class of quinazolines having metalloprotease-13 inhibitory activity. The compounds are stated to have a variety of therapeutic uses including the treatment of cancer.

WO 02/102793 (Warner-Lambert) discloses quinazolinediones as antibacterial agents.

WO 2004/014893 (Procter & Gamble) discloses antimicrobial aza-bicyclic compounds.

WO 98/10767 discloses quinazolinones as chemical intermediates in the preparation of 4-phenylaminoquinazolines.

EP 373891 (ICI) and GB 2271111 (Zeneca) each disclose a class of substituted arylaminomethyl quinazolinone compounds as anti-tumour agents.

U.S. Pat. No. 5,294,617 and EP 0497150 (American Cyanamid) each disclose a class of 2-alkylquinazolinones having angiotensin II antagonist activity. The compounds are described as being useful in treating hypertension and congestive heart failure.

JP 01061468 (Otsuka) discloses benzo-fused heterocyclic compounds for use in treating heart disease.

U.S. Pat. No. 5,441,959 describes a class of substituted phenylbenzylquinazolinones as angiotensin II antagonists. Quinazolinones having an alkylureido susitutent at the 6-position of the quinazolinone ring are disclosed as synthetic intermediates.

WO 2004/111009 (Abbott) discloses a class of fused heterocyclic compounds as vanilloid receptor antagonists.

WO 03/055492 (AstraZeneca) discloses quinazolin-4-yl-oxidoles as GSK-3 inhibitors. Quinazolinones are described as synthetic intermediates.

WO 2004/094410 and WO 2004/058781 (both to AstraZeneca) discloses 4-substituted quinazolines as anti-cancer compounds. Quinazolinones are described as synthetic intermediates.

WO 02/16362 (Cor Therapeutics) discloses substituted 4-piperazinylquinazolines as inhibitors of kinase phosphorylation. The compounds are stated to be useful as inter alia anti-cancer agents. Quinazolinones are disclosed as synthetic intermediates.

U.S. Pat. No. 5,994,542 (Sumitomo) describes a process for making quinazolinediones.

EP 1477 481 (Ube) describes a process for making quinazolinones.

WO 02/064572 (Warner-Lambert) discloses quinazolines as MMP-13 inhibitors that may be useful in the treatment of various diseases such as cancer.

### SUMMARY OF THE INVENTION

The invention provides compounds that have protein kinase A (PKA) and/or protein kinase B (PKB) inhibiting or modulating activity, and which it is envisaged will be useful in preventing or treating disease states or conditions mediated by PKA and/or PKB.

Accordingly, in one aspect, the invention provides a compound for use in the treatment or prophylaxis of a disease state or condition mediated by protein kinase A and/or protein kinase B, the compound being a compound of the formula (I):

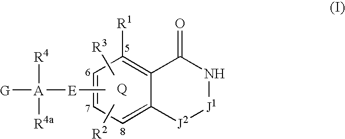

or a salt, solvate, tautomer or N-oxide thereof, wherein:
the ring Q is a benzene ring;
J²-J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷; provided that when J²-J¹ is a group R^{1a}N—CO, E is other than NR⁷CO;
m and n are each 0 or 1, provided that the sum of m and n is 1 or 2;
   A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; NH₂; NHCOR¹⁰ and NHCONHR¹⁰;
R⁴ is hydrogen or C₁₋₄ alkyl;
R^{4a} is hydrogen, C₁₋₄ alkyl or a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N;
R¹⁰ is phenyl or benzyl each optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

In another aspect, the invention provides a compound for use in the treatment or prophylaxis of a disease state or condition mediated by protein kinase B, the compound being a compound of the formula (I⁰):

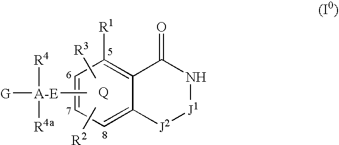

or a salt, solvate, tautomer or N-oxide thereof, wherein:
the ring Q is a benzene ring;
J²-J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷; provided that when J²-J¹ is a group R^{1a}N—CO, E is other than NR⁷CO;
m and n are each 0 or 1, provided that the sum of m and n is 1 or 2;
   A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; NH₂; NHCOR¹⁰ and NHCONHR¹⁰;
R⁴ is hydrogen or C₁₋₄ alkyl;
R^{4a} is hydrogen, C₁₋₄ alkyl or a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N;
R¹⁰ is phenyl or benzyl each optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}; and provided that when A is a bond, G and E combine to form a group R⁶R⁵NC(O)NH— attached to the ring Q at the position marked with the numeral 7; and that when G is OH, A is other than a bond and R^{4a} is R⁹.

The invention also provides novel compounds of the formula (I).

One particular group of novel compounds of the invention is the group of compounds having the formula (Ia):

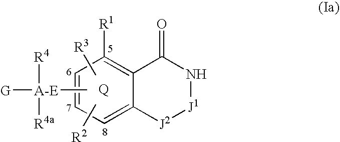

or salts, solvates, tautomers or N-oxides thereof, wherein:
the ring Q is a benzene ring;
J²-J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷; whereby when J²-J¹ is a group R^{1a}N—CO, E is other than NR⁷CO;
m and n are each 0 or 1, provided that the sum of m and n is 1 or 2;
A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
the moiety A-E having a minimum chain length of 2 atoms extending between the ring Q and the nitrogen or oxygen atom of the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; and NH₂; provided that when A is a bond and E is CONR⁷, R² is attached to the carbon atom designated by the numeral 8 on the benzene ring Q;
R⁴ is hydrogen or C₁₋₄ alkyl;
R^{4a} is a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N; and provided that:
(a) when J²-J¹ is a group R^{1a}N—CO, E is a linking atom or group E′ selected from CH═CH, (X′)ₘ(CH₂)ₙ where X is selected from O and S; and/or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
(b) when A is a bond, G and E combine to form a group R⁶R⁵NC(O)NH-attached to the ring Q at the position marked with the numeral 7, wherein at least one of R⁵ and R⁶ is other than hydrogen;
(c) when R⁴ together with R⁷ and the intervening atoms of the groups A and E form a piperidine ring and G is NR⁵R⁶ attached directly to the 3-position of the piperidine ring, then R^{4a} is other than cycloalkyl;
(d) when J²-J¹ is a group N═C(Me), the moiety R⁶R⁵N-A(R⁴)(R^{4a})-E- is other than a 2-phenyl-3-hydroxypropyl group attached to the ring Q at the carbon atom marked by the numeral 6;
(e) when G is OH and J²-J¹ is a group N═CR⁷, then R⁷ is other than an alkyl group having three or more carbon atoms;
(f) when one of R⁵ and R⁶ together with the nitrogen atom to which they are attached and R⁷ and the intervening atoms of the groups A and E form a saturated monocyclic heterocyclic group, then J²-J¹ is other than a group HN—CO;
(g) when E is (X)ₘ(CR⁸R^{8a})ₙ, m is 0 and n is 1; then J²-J¹ is other than a group HN—CO; and
(h) when the moiety R⁶R⁵N-A(R⁴)(R^{4a})-E- is a 2-morpholinoethoxy group, then J²-J¹ is other than a group HN—CO.

The invention also provides:
A compound per se of the formula (II), (III), (IV), (V) and (VI) or any other sub-group or embodiment of the formula (I) or (Ia) as defined herein.
A compound of the formula (Ia), (II), (III), (IV), (V) and (VI) or any sub-group or embodiment thereof as defined herein for use in the prophylaxis or treatment of a disease state or condition mediated by protein kinase B.
The use of a compound of formula (I), (I⁰), (Ia), (II), (III), (IV), (V) and (VI) or any sub-group or embodiment thereof as defined herein for the manufacture of a medicament for the prophylaxis or treatment of a disease state or condition mediated by protein kinase B.
A method for the prophylaxis or treatment of a disease state or condition mediated by protein kinase B, which method comprises administering to a subject in need thereof a compound of the formula (I), (10), (Ia), (II), (III), (IV), (V) and (VI) or any sub-group or embodiment thereof as defined herein.
A method for treating a disease or condition comprising or arising from abnormal cell growth or abnormally arrested cell death in a mammal, the method comprising administering to the mammal a compound of the formula (I), (I⁰), (Ia), (II), (III), (IV), (V) and (VI) or any sub-group or embodiment thereof as defined herein in an amount effective to inhibit protein kinase B activity.
A method of inhibiting protein kinase B, which method comprises contacting the kinase with a kinase-inhibiting compound of the formula (I), (I⁰), (Ia), (II), (III), (IV), (V) and (VI) or any sub-group or embodiment thereof as defined herein.
A method of modulating a cellular process (for example cell division) by inhibiting the activity of a protein kinase B using a compound of the formula (I), (I⁰), (Ia), (II), (III), (IV), (V) and (VI) or any sub-group or embodiment thereof as defined herein.
A compound of the formula (I), (I⁰), (Ia), (II), (III), (IV), (V) and (VI) or any sub-group or embodiment thereof as defined herein for use in the prophylaxis or treatment of a disease state or condition mediated by protein kinase A.
The use of a compound of formula (I), (I⁰), (Ia), (II), (III), (IV), (V) and (VI) or any sub-group or embodiment thereof as defined herein for the manufacture of a medicament for the prophylaxis or treatment of a disease state or condition mediated by protein kinase A.
A method for the prophylaxis or treatment of a disease state or condition mediated by protein kinase A, which method comprises administering to a subject in need thereof a compound of the formula (I), (I⁰), (Ia), (II), (III), (IV), (V) and (VI) or any sub-group or embodiment thereof as defined herein.
A method for treating a disease or condition comprising or arising from abnormal cell growth or abnormally arrested cell death in a mammal, the method comprising administering to the mammal a compound of the formula (I), (I⁰), (Ia), (II), (III), (IV), (V) and (VI) or any sub-group or embodiment thereof as defined herein in an amount effective to inhibit protein kinase A activity.
A method of inhibiting protein kinase A, which method comprises contacting the kinase with a kinase-inhibiting compound of the formula (I), (I⁰), (Ia), (II), (III), (IV), (V) and (VI) or any sub-group or embodiment thereof as defined herein.
A method of modulating a cellular process (for example cell division) by inhibiting the activity of a protein kinase A using a compound of the formula (I), (I⁰), (Ia), (II), (III), (IV), (V) and (VI) or any sub-group or embodiment thereof as defined herein.
The use of a compound of the formula (I), (I⁰), (Ia), (II), (III), (IV), (V) and (VI) or any sub-group or embodiment thereof as defined herein for the manufacture of a medicament for the prophylaxis or treatment of a disease state or condition arising from abnormal cell growth or abnormally arrested cell death.
A method for treating a disease or condition comprising or arising from abnormal cell growth or abnormally arrested cell death in a mammal, which method comprises administering to the mammal a compound of the formula (I), (I⁰), (Ia), (II), (III), (IV), (V) and (VI) or any sub-group or embodiment thereof as defined herein in an amount effective in inhibiting abnormal cell growth.
A method for alleviating or reducing the incidence of a disease or condition comprising or arising from abnormal cell growth or abnormally arrested cell death in a mammal, which method comprises administering to the mammal a compound of the formula (I), (I⁰), (Ia), (II), (III), (IV), (V) and (VI) or any sub-group or embodiment thereof as defined herein in an amount effective in inhibiting abnormal cell growth.
A pharmaceutical composition comprising a novel compound of the formula (I), (I⁰), (Ia), (II), (III), (IV), (V) and (VI) or any sub-group or embodiment thereof as defined herein and a pharmaceutically acceptable carrier.
A compound of the formula (I), (Ia), (II), (III), (IV), (V) and (VI) or any sub-group or embodiment thereof as defined herein for use in medicine.
The use of a compound of the formula (I), (I⁰), (Ia), (II), (III), (IV), (V) and (VI) or any sub-group or embodiment thereof as defined herein for the manufacture of a medicament for the prophylaxis or treatment of any one of the disease states or conditions disclosed herein.
A method for the treatment or prophylaxis of any one of the disease states or conditions disclosed herein, which method comprises administering to a patient (e.g. a patient in need thereof) a compound (e.g. a therapeutically effective amount) of the formula (I), (I⁰), (Ia), (II), (III), (IV), (V) and (VI) or any sub-group or embodiment thereof as defined herein.
A method for alleviating or reducing the incidence of a disease state or condition disclosed herein, which method comprises administering to a patient (e.g. a patient in need thereof) a compound (e.g. a therapeutically effective amount) of the formula (I), (I⁰), (Ia), (II), (III), (IV), (V) and (VI) or any sub-group or embodiment thereof as defined herein.
A method for the diagnosis and treatment of a disease state or condition mediated by protein kinase B, which method comprises (i) screening a patient to determine whether a disease or condition from which the patient is or may be suffering is one which would be susceptible to treatment with a compound having activity against protein kinase B; and (ii) where it is indicated that the disease or condition from which the patient is thus susceptible, thereafter administering to the patient a compound of the formula (I), (I⁰), (Ia), (II), (III), (IV), (V) and (VI) or any sub-group or embodiment thereof as defined herein.
The use of a compound of the formula (I), (I⁰), (Ia), (II), (III), (IV), (V) and (VI) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase B.
A method for the diagnosis and treatment of a disease state or condition mediated by protein kinase A, which method comprises (i) screening a patient to determine whether a disease or condition from which the patient is or may be suffering is one which would be susceptible to treatment with a compound having activity against protein kinase A; and (ii) where it is indicated that the disease or condition from which the patient is thus susceptible, thereafter administering to the patient a compound of the formula (I), (I⁰), (Ia), (II), (III), (IV), (V) and (VI) or any sub-group or embodiment thereof as defined herein.
The use of a compound of the formula (I), (I⁰), (Ia), (II), (III), (IV), (V) and (VI) or any sub-group or embodiment thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against protein kinase A.

Where they do not already apply, any one or more of the following optional provisos may apply, in any combination, to formulae (I), (I⁰), (Ia), (II), (III), (IV), (v) and (VI) or any sub-group or embodiment thereof as defined herein:
(a) When J²-J¹ is a group R^{1a}N—CO, E is a linking atom or group E′ selected from CH═CH, (X′)ₘ(CH₂)ₙ where X is selected from O and S; and/or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N.
(b) When A is a bond, G and E combine to form a group R⁶R⁵NC(O)NH— attached to the ring Q at the position marked with the numeral 7.
(c) When R⁴ together with R⁷ and the intervening atoms of the groups A and E form a piperidine ring and G is NR⁵R⁶ attached directly to the 3-position of the piperidine ring, then R^{4a} is other than cycloalkyl.
(d) When J²-J¹ is a group N═C(Me), the moiety R⁶R⁵N-A(R⁴)(R^{4a})-E- is other than a 2-phenyl-3-hydroxypropyl group attached to the ring Q at the carbon atom marked by the numeral 6.
(e) A when G is OH and J²-J¹ is a group N═CR⁷, then R⁷ is other than an alkyl group having three or more carbon atoms.
(f) when one of R⁵ and R⁶ together with the nitrogen atom to which they are attached and R⁷ and the intervening atoms of the groups A and E form a saturated monocyclic heterocyclic group, then J²-J¹ is other than a group HN—CO.
(g) when E is (X)ₘ(CR⁸R^{8a})ₙ, m is 0 and n is 1; then J²J¹ is other than a group HN—CO.
(h) when the moiety R⁶R⁵N-A(R⁴)(R^{4a})-E- is a 2-morpholinoethoxy group, then J²-J¹ is other than a group HN—CO.
(i) When J²-J¹ is a group R^{1a}N—CO, E is a linking atom or group E′ selected from CH═CH, (X′)ₘ(CH₂)ₙ where X is selected from O and S; and/or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N.
(j) When A is a bond, G and E combine to form a group R⁶R⁵NC(O)NH— attached to the ring Q at the position marked with the numeral 7, wherein at least one of R⁵ and R⁶ is other than hydrogen.
(k) When R⁴ together with R⁷ and the intervening atoms of the groups A and E form a piperidine ring and G is NR⁵R⁶ attached directly to the 3-position of the piperidine ring, then R^{4a} is other than cycloalkyl.
(l) The moiety GA(R⁴)(R^{4a})E does not contain a substituted cyclohexene group
(m) The moiety GA(R⁴)(R^{4a})E is other than a hydroxyalkyl group or a hydroxyalkoxy group.
(n) When G is OH, A is other than a bond and R^{4a} is a group R⁹. (EP 1044969)
(o) When a saturated monocyclic 4-7 membered heterocyclic group is formed by (i) R⁴ together with R⁷ or R⁸ and the intervening atoms of the groups A and E; or (ii) one of R⁵ and R⁶ together with the nitrogen atom to which they are attached and R⁷ or R⁸ and the intervening atoms of the groups A and E; the saturated monocyclic 4-7 membered heterocyclic group is other than a five membered ring containing an oxygen ring member.
(p) When J²-J¹ is a group R^{1a}N—CO, E is (X)ₘ(CR⁸R^{8a})ₙ where m is 1, n is 0 and X is NR⁷, and a saturated monocyclic 4-7 membered heterocyclic group is formed by R⁴ together with R⁷ and the intervening atoms of the groups A and E, then the saturated monocyclic 4-7 membered heterocyclic group is other than an optionally substituted pyrrolidine or azetidine group.

### General Preferences and Definitions

The following general preferences and definitions shall apply to each of the moieties A, E, J¹, J² and R¹ to R¹⁰ and any sub-definition, sub-group or embodiment thereof, unless the context indicates otherwise.

Any references to Formula (I) herein shall be taken also to refer to formulae formula (I⁰), (Ia), (II), (III), (IV), (V) and (VI) and any other sub-group of compounds within formula (I) unless the context requires otherwise.

In this specification, references to “the quinazolinone group”, when used in regard to the point of attachment of the group E shall, unless the context indicates otherwise, be taken to refer to the group:

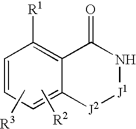

References to “carbocyclic” and “heterocyclic” groups as used herein shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

One sub-group of bicyclic heteroaryl groups consists of groups a) to e) and g) to o) above.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups include unsubstituted or substituted (by one or more groups R¹¹) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1,2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

Examples of monocyclic non-aromatic heterocyclic groups include 5-, 6- and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹¹) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹¹ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

Where the substituent group R¹¹ comprises or includes a carbocyclic or heterocyclic group, the said carbocyclic or heterocyclic group may be unsubstituted or may itself be substituted with one or more further substituent groups R¹¹. In one sub-group of compounds of the formula (I), such further substituent groups R¹¹ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹¹.

The substituents R¹¹ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

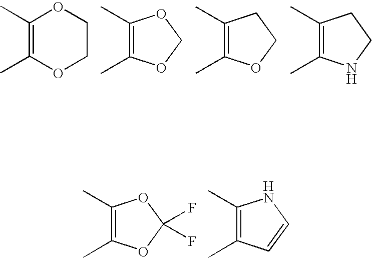

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated.

In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or, where stated, can be substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl(allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl, naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}CRC)O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### Specific Embodiments of and Preferences for A, E, G, J¹, J² and R¹ to R¹¹

In formulae (I) and (Ia), J²-J¹ is a group N═CH or a group R^{1a}N—CO.

In one preferred embodiment, J²-J¹ is a group N═CH and hence the compounds of the formula (I) are quinazolinones.

In another embodiment, J²-J¹ is a group R^{1a}N—CO wherein R^{1a} is selected from hydrogen; C₁₋₆ hydrocarbyl optionally substituted by halogen, hydroxy or C₁₋₂ alkoxy; CONHR⁸; NH₂; NHCOR¹⁰ and NHCONHR¹⁰.

More typically, R^{1a} is selected from hydrogen and C₁₋₃ saturated hydrocarbyl and more particularly from hydrogen, methyl and ethyl. Preferably R^{1a} is selected from hydrogen and methyl, and more preferably is hydrogen.

G is OH or NR⁵R⁶. In one particular group of compounds, G is NR⁵R⁶. In another particular group of compounds, G is OH.

A can be a bond and R⁴ and R^{4a} are absent or A can be a saturated hydrocarbon linker group containing from 1 to 7 carbon atoms, the linker group having a maximum chain length of 5 atoms extending between E and G.

In one sub-group of compounds, A is a saturated hydrocarbon linker group containing from 1 to 7 carbon atoms, the linker group having a maximum chain length of 5 atoms extending between E and G. The moieties G, R⁴, R^{4a} and E can each be attached at any location on the group A.

In formula (I), in one embodiment, the moiety A-E may have a minimum chain length of 2 atoms extending between the ring Q and the nitrogen or oxygen atom of the group G.

In formula (Ia), the moiety A-E has a minimum chain length of 2 atoms extending between the ring Q and the nitrogen atom of the group G.

The terms “maximum chain length” and “minimum chain length” as used herein refers to the number of atoms lying directly between the two moieties in question, and does not take into account any branching in the chain or any hydrogen atoms that may be present. For example, in the structure A shown below:

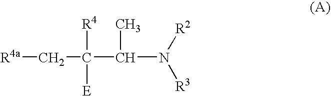

the chain length between E and NR⁵R⁶ is 2 atoms.

In the structure (B) below, the chain length between the ring Q and the nitrogen atom of the group NR⁵R⁶ is 5 atoms.

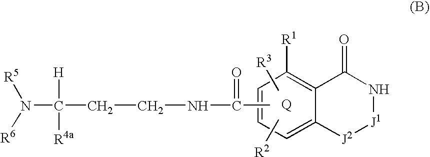

It is preferred that the linker group has a maximum chain length of 4 atoms, more typically 3 atoms, extending between E and G.

When R^{4a} is a group R⁹, the linker group typically has a maximum chain length of 4 atoms (for example up to 3 atoms, e.g. 1, 2, or 3), and more preferably 3 atoms) extending between R⁹ and G.

In one particular group of compounds, the linker group has a chain length of 3 atoms extending between R⁹ and G and a chain length of 3 or 4 atoms (preferably 3 atoms) extending between E and G.

One of the carbon atoms in the linker group may optionally be replaced by an oxygen or nitrogen atom.

When a nitrogen atom or oxygen atom are present, it is preferred that the nitrogen or oxygen atom and the G group are spaced apart by at least two intervening carbon atoms.

In one particular group of compounds within formula (I), the linker atom linked directly to the group E is a carbon atom and the linker group A has an all-carbon skeleton.

In one embodiment, for example, the linker group A, taken together with R⁴, R^{4a}, E and NR⁵R⁶, can have the structure:

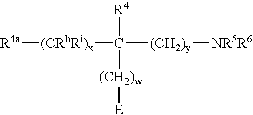

wherein R^{h} and Rⁱ are the same or different and each is selected from hydrogen, methyl and fluorine, w is 0 or 1, x is 0 to 3 and y is 0 to 3, provided that the total of w, x and y added to the number of carbon atoms in R⁴ does not exceed 7; and R⁴ is hydrogen or C₁₋₄ alkyl; or R⁴ and R⁵ are linked so that the moiety R⁴—C—(CH₂)_{y}—NR⁵R⁶ forms a 4-7 membered ring.

The carbon atoms of the linker group A may optionally bear one or more substituents selected from oxo, fluorine and hydroxy, provided that the hydroxy group and oxo group are not located at a carbon atom a with respect to the G group. Typically, the hydroxy group, if present, is located at a position β with respect to the G group. In general, no more than one hydroxy group will be present. Where fluorine atoms are present, they may be present in a difluoromethylene or trifluoromethyl group, for example.

In one embodiment of the invention, no fluorine atoms are present in the linker group A.

In another embodiment of the invention, no hydroxy groups are present in the linker group A.

In a further embodiment, no oxo group is present in the linker group A.

In one group of compounds of the formula (I) neither hydroxy groups nor fluorine atoms are present in the linker group A, e.g. the linker group A is unsubstituted.

Preferably, when a carbon atom in the linker group A is replaced by a nitrogen atom, the group A bears no more than one hydroxy substituent and more preferably bears no hydroxy substituents.

In order to modify the susceptibility of the compounds to metabolic degradation in vivo, the linker group A can have a branched configuration at the carbon atom attached to the G group. For example, the carbon atom attached to the G group can be attached to a pair of gem-dimethyl groups.

In another sub-group of compounds of the invention, A is a bond and R⁴ and R^{4a} are absent.

In a preferred group of compounds of the invention, G is NR⁵R⁶ and R⁵ and R⁶ are each selected from hydrogen, a group R⁹ and C₁₋₄ hydrocarbyl (e.g. saturated hydrocarbyl) optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;

In one group of compounds of the invention, R⁵ and R⁶ are independently selected from hydrogen and saturated C₁₋₄ hydrocarbyl. Typically the hydrocarbyl group is an alkyl group, more usually a C₁, C₂ or C₃ alkyl group, for example a methyl group. In one particular sub-group of compounds, R⁵ and R⁶ are independently selected from hydrogen and methyl and hence NR⁵R⁶ can be an amino, methylamino or dimethylamino group. More particularly, NR⁵R⁶ can be an amino group.

In another group of compounds, R⁵ and R⁶ together with the nitrogen atom to which they are attached form a saturated monocyclic heterocyclic group having 4-7 ring members and optionally containing a second heteroatom ring member selected from O and N.

The saturated monocyclic ring can be an azacycloalkyl group such as an azetidine, pyrrolidine, piperidine or azepane ring, and such rings are typically unsubstituted. Alternatively, the saturated monocyclic ring can contain an additional heteroatom selected from O and N, and examples of such groups include morpholine and piperazine. Where an additional N atom is present in the ring, this can form part of an NH group or an N—C₁₋₄alkyl group such as an N-methyl, N-ethyl, N-propyl or N-isopropyl group.

In another sub-group of compounds of the invention, one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N. Such groups are typically unsubstituted.

Examples of such compounds include compounds wherein R⁴, NR⁵R⁶ and A form a unit of the formula:

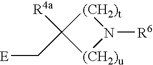

where t and u are each 0, 1, 2 or 3 provided that the sum of t and u falls within the range of 2 to 5, e.g. 2 to 4, and preferably 4.

Further examples of such compounds include compounds wherein R⁴, NR⁵R⁶ and A form a group of the formula:

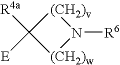

where v and w are each 0, 1, 2 or 3 provided that the sum of v and w falls within the range of 2 to 5. Particular examples of such compounds are those in which v and w are both 2.

In a further group of compounds, one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N. Such groups are typically unsubstituted.

Examples of such compounds include compounds wherein NR⁵R⁶, R⁸, E and A form a group of the formula:

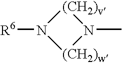

where v′ and w′ are each 2 or 3 provided that the sum of v and w falls within the range of 4 to 5.

In another sub group of compounds, R⁴ and R^{4a} together with the intervening atom or atoms of the group A form a saturated monocyclic heterocyclic group having 4-7 ring members and optionally containing a second heteroatom ring member selected from O and N. Such groups are typically unsubstituted.

Examples of such compounds are compounds where R⁴, R^{4a}, R⁸ and A form a group of the formula:

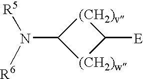

where v″ and w″ are each 0, 1, 2 or 3 provided that the sum of v″ and w″ falls within the range of 1 to 5.

In another group of compounds, R⁴ together with R⁷ or R⁸ and the intervening atoms of the groups A and E form a saturated monocyclic heterocyclic group having 4-7 ring members and optionally containing a second heteroatom ring member selected from O and N. Such groups are typically unsubstituted.

Examples of such compounds are compounds where R⁴, R⁸, E, NR⁵R⁶ and A form a group of the formula:

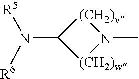

where v″ and w″ are each 0, 1, 2 or 3 provided that the sum of v″ and w″ falls within the range of 2 to 5.

Further examples of such compounds are compounds where R⁴, R⁸, E and A form a group of the formula:

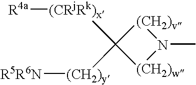

where v″ and w″ are each 0, 1, 2 or 3 provided that the sum of v″ and w″ falls within the range of 2 to 5; y′ is 0, 1 or 2 and x′ is 0, 1 or 2, and R^{j} and R^{k} are the same or different and each is selected from hydrogen, methyl and fluorine.

Preferably, x′ and y′ are each independently 0 or 1 and, in one particular embodiment, x′ and y′ are both 1.

Particular examples of the moiety A-E, together with their points of attachment to the groups R⁴, R^{4a} and G, are shown in Table 1 below. The point of attachment to the quinazolinone group is indicated in the formulae in Table 1 by means of an asterisk.

TABLE 1

| | |
|---|---|
| 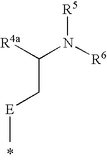 | 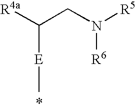 |
| A1 | A2 |
| 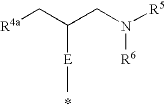 | 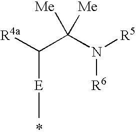 |
| A3 | A4 |
| 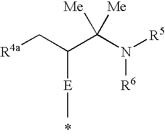 | 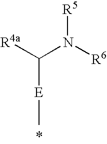 |
| A5 | A6 |
| 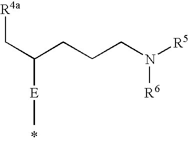 | 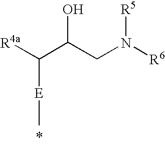 |
| A7 | A8 |
| 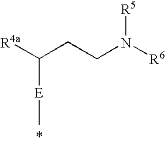 | 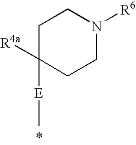 |
| A9 | A10 |
| 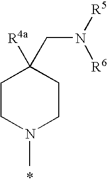 | 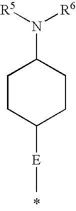 |
| A11 | A12 |
| 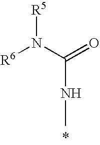 | 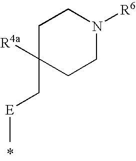 |
| A13 | A14 |
| 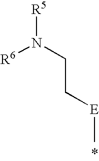 | 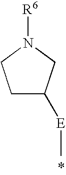 |
| A15 | A16 |
| 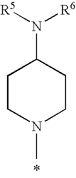 | 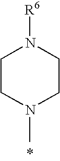 |
| A17 | A18 |
| 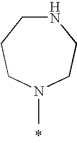 | 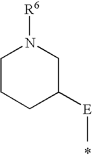 |
| A19 | A20 |
| 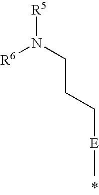 | 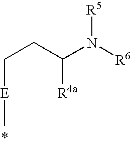 |
| A21 | A22 |
| 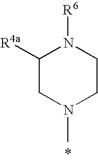 | 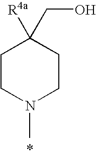 |
| A23 | A24 |
| 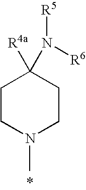 | 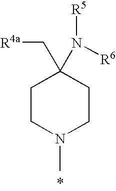 |
| A25 | A26 |
| 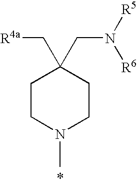 | 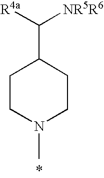 |
| A27 | A28 |

In formulae A11, A17, A18, A19, A23 and A24, the group E forms part of the cyclic structure and in formula A13, the group E forms part of the urea group.

One sub-set of preferred groups includes A9, A10, A11 and A14.

Another subset of preferred groups includes A9, A10, A11, A14 and A27.

The group R⁴ is selected from hydrogen and C₁₋₄ alkyl. In one embodiment, R⁴ is hydrogen. In another embodiment, R⁴ is methyl.

The group R^{4a} is selected from hydrogen, C₁₋₄ alkyl and a group R⁹ where R⁹ is as defined herein.

In one sub-group of compounds, R^{4a} is a group R⁹.

In another sub-group of compounds, R^{4a} is hydrogen or C₁₋₄ alkyl.

When R^{4a} is a group R⁹, the carbocyclic group or heterocyclic group may be selected from the list of such groups set out in the section headed General Preferences and Definitions.

In one embodiment, the carbocyclic group or heterocyclic group is an aryl or heteroaryl group.

Thus, R⁹ can be monocyclic or bicyclic and, in one particular embodiment, is monocyclic. Particular examples of monocyclic aryl and heteroaryl groups are six membered aryl and heteroaryl groups containing up to 2 nitrogen ring members, and five membered heteroaryl groups containing up to 3 heteroatom ring members selected from O, S and N.

Examples of such groups include phenyl, naphthyl, thienyl, furan, pyrimidine and pyridine, with phenyl being presently preferred.

The aryl or heteroaryl group R⁹ can be unsubstituted or substituted by up to 5 substituents, and examples of substituents are those listed in group R¹¹ above. Particular substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R⁹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R⁹ can have one or two substituents selected from fluorine, chlorine, trifluoromethyl, methyl and methoxy. When R⁹ is a phenyl group, particular examples of substituent combinations include mono-chlorophenyl and dichlorophenyl.

When R⁹ is a six membered aryl or heteroaryl group, a substituent may advantageously be present at the para position on the six-membered ring. Where a substituent is present at the para position, it may be, for example, larger in size than a fluorine atom.

The group E is a linking atom or group selected from CONR⁷, NR⁷CO, C(R⁸)═C(R⁸), (X)ₘ(CR⁸R^{8a})ₙ where X is selected from O, S and NR⁷ and m and n are each 0 or 1, provided that the sum of m and n is 1 or 2. In the foregoing list of groups E, the left hand side of each group is attached to the moiety A whereas the right hand side of each group is attached to the benzene ring Q.

In one embodiment, E is selected from CONR⁷ and NR⁷CO. One group of preferred compounds is the group in which R⁷ is hydrogen.

When E is (X)ₘ(CR⁸R^{8a})ₙ, m can be 0 in which case E is CR⁸R^{8a}, or n can be 0 in which case E is X, or m and n are each 1 in which case E is XCR⁸R^{8a}.

In another embodiment, E is NH.

In a further embodiment, E is O.

In a still further embodiment, E is CH₂.

In another embodiment, E is CH═CH, preferably trans CH═CH.

R¹, R^{1a}, R², and R³ are each independently selected from hydrogen; halogen; C₁₋₆ hydrocarbyl (e.g. saturated hydrocarbyl) optionally substituted by halogen, hydroxy or C₁₋₂ alkoxy; cyano; CONH₂; CONHR⁸; CF₃; NH₂; NHCOR¹⁰ and NHCONHR¹⁰.

More typically, R¹ is selected from hydrogen, chlorine, fluorine, C₁₋₃ saturated hydrocarbyl, cyano, CF₃ and CONH₂, and more particularly from hydrogen, chlorine, fluorine, methyl, cyano and CF₃. In one embodiment, R¹ is hydrogen.

More typically R² and R³ are each independently selected from hydrogen, halogen, C₁₋₅ saturated hydrocarbyl, cyano, CF₃, CONH₂, CONHR⁸ and NH₂. For example, R² and R³ may be selected from hydrogen, halogen, C₁₋₅ saturated hydrocarbyl, cyano and CF₃, more typically hydrogen, chlorine, fluorine, C₁₋₃ saturated hydrocarbyl, cyano and CF₃. In one embodiment, one or both of R² and R³ are hydrogen.

In a particular embodiment of the invention, R¹, R² and R³ each are hydrogen.

In formula (I), R⁴ is selected from hydrogen, halogen, C₁₋₅ saturated hydrocarbyl, cyano and CF₃. Preferred values for R⁴ include hydrogen and methyl.

The group R¹⁰ when present is selected from phenyl and benzyl each optionally substituted as defined herein. Particular groups R¹⁰ are phenyl and benzyl groups that are unsubstituted or are substituted with a solubilising group such as an alkyl or alkoxy group bearing an amino, substituted amino, carboxylic acid or sulphonic acid group. Particular examples of solubilising groups include amino-C₁₋₄-alkyl, mono-C₁₋₂-alkylamino-C₁₋₄-alkyl, di-C₁₋₂-alkylamino-C₁₋₄-alkyl, amino-C₁₋₄-alkoxy, mono-C₁₋₂-alkylamino-C₁₋₄-alkoxy, di-C₁₋₂-alkylamino-C₁₋₄-alkoxy, piperidinyl-C₁₋₄-alkyl, piperazinyl-C₁₋₄-alkyl, morpholinyl-C₁₋₄-alkyl, piperidinyl-C₁₋₄-alkoxy, piperazinyl-C₁₋₄-alkoxy and morpholinyl-C₁₋₄-alkoxy.

The group R⁵R⁶N-A(R⁴)(R^{4a})-E- can be linked to any one of the 6, 7 or 8 positions of the quinazolinone group.

One sub-group of compounds within formulae (I) and (Ia) is the group of compounds of formula (II):

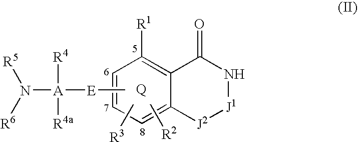

or salts, solvates, tautomers or N-oxides thereof.

Within formula (II) are compounds of the formula (IIa):

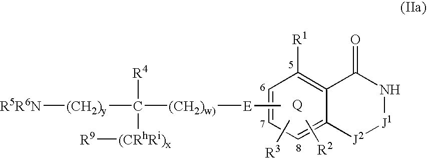

where R^{h} and Rⁱ are the same or different and each is selected from hydrogen, methyl and fluorine, w is 0 or 1, x is 0 to 3 and y is 0 to 3, provided that the total of w, x and y added to the number of carbon atoms in R⁴ does not exceed 7; and R⁴ is hydrogen or C₁₋₄ alkyl; or R⁴ and R⁵ are linked so that the moiety R⁴—C—(CH₂)_{y}—NR⁵R⁶ forms a saturated 4-7 membered ring; and E, R¹, R², R³, R⁹ and

J²-J¹ are as defined herein.

In one embodiment of formula (IIa), R⁴ and R⁵ are not linked. Within this embodiment:
R⁴ is preferably hydrogen or methyl (and more preferably is hydrogen) and/or:
w is preferably 0 or 1; and/or
x is preferably 0, 1 or 2; more preferably 0 or 1; and/or
y is preferably 0, 1 or 2; more preferably 1 or 2 and/or
E is selected from CONR^{7a}, NR^{7a}Co, C(R^{8b})═C(R^{8b}), NR^{7a}, and O where R^{7a} and R^{8a} are each selected from hydrogen and methyl, and more preferably are each hydrogen; particular examples of E being CONH, NHCO, NH, O and CH═CH (e.g. trans CH═CH).

In another embodiment of formula (IIa), R⁴ and R⁵ are linked so that the moiety R⁴—C—(CH₂)_{y}—NR⁵R⁶ forms a 4-7 membered ring. Within this embodiment:
the saturated 4-7 membered ring is preferably a 5- or 6-membered ring (and more preferably a 6-membered ring) typically containing only a single heteroatom which is the nitrogen atom of the group NR⁵R⁶; and/or
preferably R⁴ and R⁵ link together to form a group —(CH₂)ₙ— where v is 1, 2 or 3 and y is 1, 2 or 3 provided that the total of v and y does not exceed 5 (and more typically does not exceed 4), and more preferably v and y are both 2; and/or
the saturated ring is optionally substituted by one or more C₁₋₄ alkyl groups such as methyl but more preferably is unsubstituted; and/or

E is selected from CONR^{7a}, NR^{7a}CO, C(R^{8b})═C(R^{8b}), NR^{7a}, and O where R^{7a} and R^{8a} are each selected from hydrogen and methyl, and more preferably are each hydrogen.

In formula (IIa) and each of its embodiments as defined above and elsewhere herein, it is preferred that:
J²-J¹ is a group N═CH and/or
the group E is preferably attached to the carbon atom numbered 7 of the ring Q; and/or
R¹, R^{1a} (if present), R² and R³ are the same or different and each is selected from hydrogen, methyl, chlorine and fluorine, and more preferably each is hydrogen; and/or
when R⁴ and R⁵ are not linked to form a ring, the group NR⁵R⁶ is selected from amino, methylamino and dimethylamino and more preferably is methylamino or amino; and when R⁴ and R⁵ are linked to form a ring, NR⁶ is NH or N-methyl.

One particular sub-group of compounds within formula (IIa) can be represented by the formula (IIb):

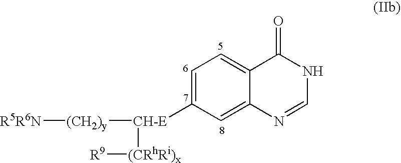

wherein R⁵, R⁶, R⁹, R^{h}, Rⁱ, x and y are as defined herein.

Another particular sub-group of compounds within formula (IIa) can be represented by the formula (IIc):

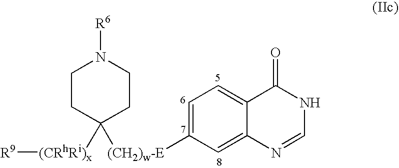

wherein R⁶, R⁹, R^{h}, Rⁱ, x and w are as defined herein.

In formula (IIc), x is typically 0 or 1. In one embodiment, x is 0. In another embodiment, x is 1. When x is 1, R^{h} and Rⁱ can each be hydrogen, fluorine or methyl. In one embodiment, R^{h} and Rⁱ are each hydrogen.

The integer w is typically 0 or 1. When E is CH═CH or CONH, wherein the nitrogen atom of the amide group is attached to the quinazolinone ring, then w is preferably 0. When E is O or NH, then w is preferably 1.

The moiety R⁶ is typically hydrogen or methyl. In one embodiment, R⁶ is hydrogen.

Also within formula (II) are compounds of the formula (IId):

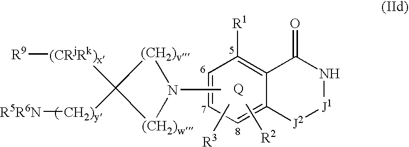

wherein v″ and w″ are each 0, 1, 2 or 3 provided that the sum of v″ and w″ falls within the range of 2 to 5; y′ is 0, 1 or 2 and x′ is 0, 1 or 2, and R^{j} and R^{k} are the same or different and each is selected from hydrogen, methyl and fluorine.

In formula (IId):
preferably, x′ and y′ are each independently 0 or 1 and, in one particular embodiment, x′ and y′ are both 1; and/or
preferably, one of v″ and w″ is 1 or 2 and the other of v″ and w″ is 2, and more preferably both of v″ and w″ are 2; and/or
preferably the nitrogen atom of the ring containing the moieties (CH₂)_{v″} and (CH₂)_{w″} is attached to the carbon atom numbered 7 in the ring Q; and/or
typically J²-J¹ is a group N═CH and/or
typically R¹, R^{1a} (if present), R² and R³ are the same or different and each is selected from hydrogen, methyl, chlorine and fluorine, and more preferably each is hydrogen; and/or
the group NR⁵R⁶ is typically selected from amino, methylamino and dimethylamino and more preferably is methylamino or amino.

One sub-group of compounds within formula (IId) can be represented by the formula (IIe):

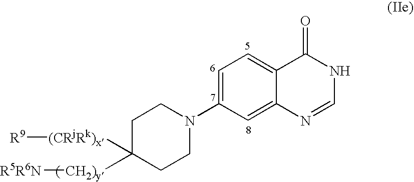

wherein R⁵, R⁶, R⁹, x′, y′, R^{j} and R^{k} are as defined herein. Preferably, x′ and y′ are each independently 0 or 1. In one embodiment, x′ is 0 and y′ is 1. In another embodiment, x′ is 1 and y′ is 0. In a further embodiment x′ is 1 and y′ is 1. In a still further embodiment, x′ and y′ are both 0.

In formulae (IIa) to (IIe) and embodiments thereof, the group R⁹ is preferably an optionally substituted aryl or heteroaryl group, and typically a monocyclic aryl or heteroaryl group of 5 or 6 ring members, particular aryl and heteroaryl groups being optionally substituted phenyl, pyridyl, furanyl and thienyl groups, with optionally substituted phenyl groups being particularly preferred.

The aryl or heteroaryl group R⁹ can be unsubstituted or substituted by up to 5 substituents, and examples of substituents are those listed in group R¹¹ above. Particular substituents include hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁₋₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

Although up to 5 substituents may be present, more typically there are 0, 1, 2, 3 or 4 substituents, preferably 0, 1, 2 or 3, and more preferably 0, 1 or 2.

In one embodiment, the group R⁹ is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

In another embodiment, the group R⁹ can have one or two substituents selected from fluorine, chlorine, trifluoromethyl, methyl and methoxy. When R⁹ is a phenyl group, particular examples of substituent combinations include mono-chlorophenyl (e.g. 4-chlorophenyl) and dichlorophenyl, (e.g. 3,4-dichlorophenyl).

Another sub-group of compounds within formula (II) is the group of compounds where J²-J¹ is a group N═CH, such compounds being represented by the formula (III):

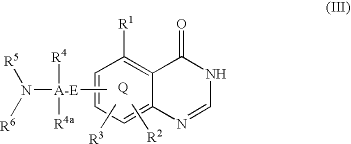

or salts, solvates, tautomers or N-oxides thereof.

Within formula (III), preferred compounds include those in which the moiety R⁵R⁶N-A(R⁴)(R^{4a})-E- is linked to the 7-position of the quinazolinone ring, i.e. compounds of the formula (IV):

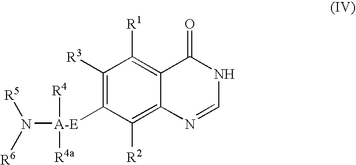

or salts, solvates, tautomers or N-oxides thereof.

Another sub-group of compounds of the formula (III) is the group of compounds where J²-J¹ is a group N═CH, the moiety R⁵R⁶N-A(R⁴)(R^{4a})-E- is linked to the 8-position of the quinazolinone ring, and A is other than a bond. Such compounds have the formula (V):

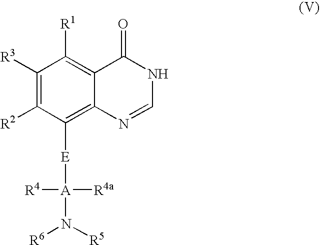

or salts, solvates, tautomers or N-oxides thereof.

In sub-groups (III) to (V), particular compounds are those wherein E is selected from CONH and HNCO.

In each of formulae (III) to (V), preferred compounds are those wherein A is a saturated hydrocarbon group.

In formulae (III), (IV) and (V), preferred values of R¹ to R⁶, A and E are as set out above in relation to formulae (II), and (IIa) to (IIe) and their embodiments.

A further sub-group of compounds within formulae (I) and (Ia) is the group of compounds of the formula (VI):

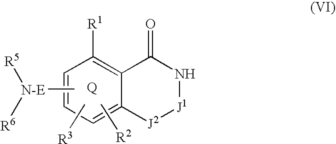

or salts, solvates, tautomers or N-oxides thereof.

Within formula (VI), particular compounds are those wherein E is a group CONH.

Another sub-group of compounds within formula (I) is the group of compounds of the formula (VII):

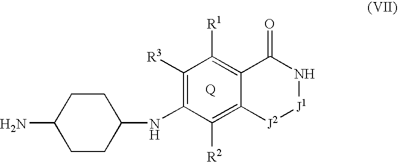

or salts, solvates, tautomers or N-oxides thereof, wherein J¹, J², R¹, R² and R³ are as defined herein.

Within formula (VII), it is preferred that:
J²-J¹ is a group N═CH and/or
R¹, R^{1a} (if present), R² and R³ are the same or different and each is selected from hydrogen, methyl, chlorine and fluorine, and more preferably each is hydrogen.

Within formula (VII), preferred compounds are those wherein the two amino groups attached to the cyclohexene ring are in the trans-relative orientation.

The specific embodiments of and preferences for A, E, G, J¹, J² and R¹ to R¹¹ set out above and below apply to each of the formulae (I), (Ia), (Ib), (Ic), (Id), (Ie), (II), (III), (IV), (V), (VI) and (VII) unless the context requires otherwise.

For the avoidance of doubt, it is to be understood that each general and specific preference, embodiment and example of the groups R¹ may be combined with each general and specific preference, embodiment and example of the groups R² and/or R³ and/or R⁴ and/or R^{4a} and/or R⁵ and/or R⁶ and/or R⁷ and/or R⁸ and/or R⁹ and/or R¹⁰ and/or R¹¹ and/or R¹² and/or G and/or A and/or E and/or J¹-J² and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are selected from:
4-amino-2-(3,4-dichloro-phenyl)-N-(4-oxo-3,4-dihydro-quinazolin-7-yl)-butyramide;
2-(4-chloro-phenyl)-4-methylamino-N-(4-oxo-3,4-dihydro-quinazolin-7-yl)-butyramide;
4-oxo-3,4-dihydro-quinazoline-7-carboxylic acid[3-amino-1-(4-chloro-phenyl)-propyl]-amide;
4-phenyl-piperidine-4-carboxylic acid (4-oxo-3,4-dihydro-quinazolin-7-yl)-amide;
7-[4-aminomethyl-4-(4-chloro-phenyl)-piperidin-1-yl]-3H-quinazolin-4-one;
(S)-4-amino-2-(3,4-dichloro-phenyl)-N-(4-oxo-3,4-dihydro-quinazolin-7-yl)butyramide;
(R)-4-amino-2-(3,4-dichloro-phenyl)-N-(4-oxo-3,4-dihydro-quinazolin-7-yl)butyramide;
1-(4-chloro-benzyl)-3-(4-oxo-3,4-dihydro-quinazolin-7-yl)-urea;
1-(4-fluoro-benzyl)-3-(4-oxo-3,4-dihydro-quinazolin-7-yl)-urea;
7-(4-phenyl-piperidin-4-ylmethoxy)-3H-quinazolin-4-one;
1-benzyl-3-(4-oxo-3,4-dihydro-quinazolin-7-yl)-urea;
7-(3-amino-propoxy)-3H-quinazolin-4-one;
7-(2-amino-ethoxy)-3H-quinazolin-4-one;
4-oxo-3,4-dihydro-quinazoline-7-carboxylic acid[3-amino-3-(4-chloro-phenyl)-propyl]-amide;
7-(2-dimethylamino-ethoxy)-3H-quinazolin-4-one;
1-(4-chloro-phenyl)-3-(4-oxo-3,4-dihydro-quinazolin-7-yl)-urea;
7-(3-amino-propyl)-3H-quinazolin-4-one;
7-(trans-4-amino-cyclohexylamino)-3H-quinazolin-4-one;
7-(pyrrolidin-3-ylamino)-3H-quinazolin-4-one;
7-(4-amino-piperidin-1-yl)-3H-quinazolin-4-one;
7-piperazin-1-yl-3H-quinazolin-4-one;
7-[1,4]diazepan-1-yl-3H-quinazolin-4-one;
7-(piperidin-3-ylamino)-3H-quinazolin-4-one;
7-(4-amino-cyclohexylamino)-1H-quinazoline-2,4-dione;
7-(trans-4-amino-cyclohexylamino)-1H-quinazoline-2,4-dione;
7-(4-methyl-[1,4]diazepan-1-yl)-3H-quinazolin-4-one;
7-[4-(4-methyl-piperazin-1-yl)-piperidin-1-yl]-3H-quinazolin-4-one;
7-(4-morpholin-4-yl-piperidin-1-yl)-3H-quinazolin-4-one;
7-(3-phenyl-piperazin-1-yl)-1H-quinazoline-2,4-dione;
7-(4-methyl-piperazin-1-yl)-3H-quinazolin-4-one;
4-(4-chloro-phenyl)-piperidine-4-carboxylic acid (4-oxo-3,4-dihydro-quinazolin-7-yl)-amide;
7-(4-amino-cyclohexyloxy)-3H-quinazolin-4-one;
7-{[4-(4-chloro-phenyl)-piperidin-4-ylmethyl]-amino}-3H-quinazolin-4-one; and
7-[4-(4-chloro-phenyl)-piperidin-4-ylmethoxy]-3H-quinazolin-4-one.

In one embodiment, the compound of the formula (I) is selected from the group consisting of:
4-amino-2-(3,4-dichloro-phenyl)-N-(4-oxo-3,4-dihydro-quinazolin-7-yl)-butyramide;
2-(4-chloro-phenyl)-4-methylamino-N-(4-oxo-3,4-dihydro-quinazolin-7-yl)-butyramide;
4-oxo-3,4-dihydro-quinazoline-7-carboxylic acid[3-amino-1-(4-chloro-phenyl)-propyl]-amide;
4-phenyl-piperidine-4-carboxylic acid (4-oxo-3,4-dihydro-quinazolin-7-yl)-amide;
7-[4-aminomethyl-4-(4-chloro-phenyl)-piperidin-1-yl]-3H-quinazolin-4-one;
(S)-4-amino-2-(3,4-dichloro-phenyl)-N-(4-oxo-3,4-dihydro-quinazolin-7-yl)butyramide;
(R)-4-amino-2-(3,4-dichloro-phenyl)-N-(4-oxo-3,4-dihydro-quinazolin-7-yl)butyramide;
4-(4-chloro-phenyl)-piperidine-4-carboxylic acid (4-oxo-3,4-dihydro-quinazolin-7-yl)-amide;
7-[4-aminomethyl-4-(4-chloro-phenyl)-piperidin-1-yl]-2-methyl-3H-quinazolin-4-one;
7-{4-[amino-(4-chloro-phenyl)-methyl]-piperidin-1-yl}-3H-quinazolin-4-one;
7-[4-(4-chloro-phenyl)-piperidin-4-ylmethoxy]-1-methyl-1H-quinazoline-2,4-dione;
7-[4-amino-4-(4-chloro-benzyl)-piperidin-1-yl]-3H-quinazolin-4-one;
7-{2-[4-(4-chloro-phenyl)-piperidin-4-yl]-vinyl}-3H-quinazolin-4-one;
7-[4-amino-4-(4-chloro-phenyl)-piperidin-1-yl]-3H-quinazolin-4-one;
7-[4-aminomethyl-4-(4-chloro-benzyl)-piperidin-1-yl]-3H-quinazolin-4-one; and
7-[4-Aminomethyl-4-(4-chloro-benzyl)-piperidin-1-yl]-1-methyl-1H-quinazoline-2,4-dione.

### Salts, Solvates Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (Ia), (II), (III), (IV), (V) and (VI) and sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, the compounds of formula (I) can exist in either of the tautomeric forms (A) and (B) and, although formula (I) is shown as being in the (A) tautomeric form, it is to be understood that formula (I) embraces both the (A) and (B) tautomers.

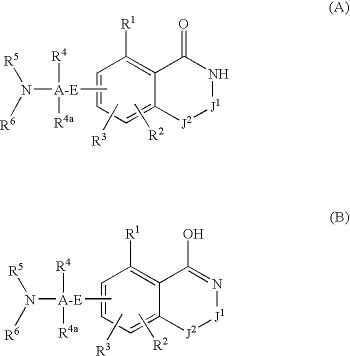

When the group J²-J¹ is N═CR⁷, formula (I) embraces both the tautomers (C) and (D) although, for simplicity, only the tautomer (C) is shown.

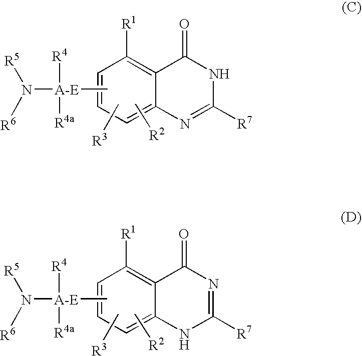

Furthermore, when the group J²-J¹ is HN—CO, Formula (I) embraces not only the amide form shown but also any imino-alcohol tautomers that may form.

Further examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

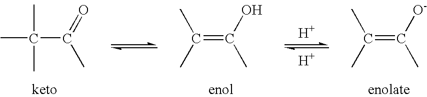

It is also to be understood that in the formulae set forth in this application, the various exclusion clauses and provisos apply to all tautomeric forms of the compounds, structures, part structures or substituent groups defined in the exclusion clauses and provisos. For example, where an exclusion clause or proviso refers to a compound wherein J²-J¹ is N═CR⁷, the exclusion clause or proviso also embraces the corresponding tautomers having the form (D) above.

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group. Examples of esters are compounds containing the group —C(—O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Methods for the Preparation of Compounds of the Formula (I)

In this section, references to Formula (I) include Formulae (Ia), (II), (III), (IV), (V) and (VI) and sub-groups thereof as defined herein unless the context requires otherwise.

The invention also provides a process for the preparation of a compound of the formula (I), which process comprises:

(a) when E is CONR⁷, the reaction of a compound of the formula (X) with a compound of the formula (X¹) or an activated derivative thereof, under amide forming conditions:

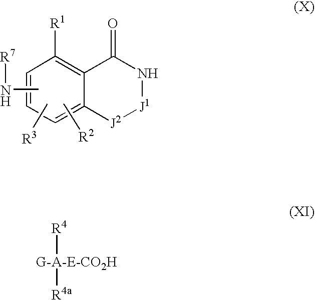

(b) when E is NR⁷CO, the reaction of a compound of the formula (XII) or an activated derivative thereof with a compound of the formula (XIII) under amide forming conditions:

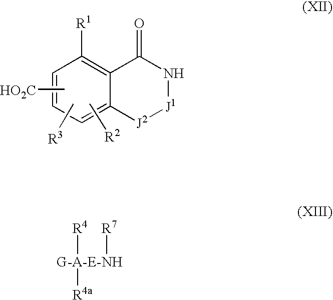

(c) when E is O or S, the reaction of a compound of the formula (XIV) or an N-protected form thereof with a compound of the formula (XV):

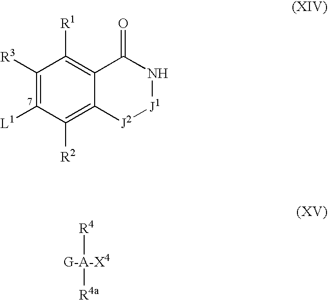

where L¹ is a leaving group or atom such as fluorine and X⁴ is OH or SH or an anion thereof in the presence of a base;
(d) when E is O or S, the reaction of a compound of the formula (XIVa) or an N-protected form thereof with a compound of the formula (XVa):

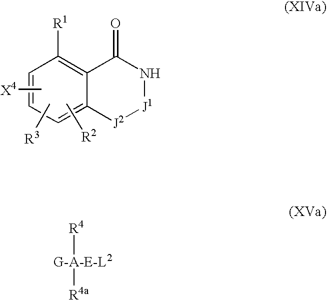

where L² is a leaving group or atom such as bromine and X⁴ is OH or SH or an anion thereof, in the presence of a base;
(e) when E is NR⁷, the reaction of a compound of the formula (XIV) with a compound of the formula (XIII), wherein (XIII) and (XIV) are as hereinbefore defined;
(f) when E is CONR⁷, A is a bond, R⁴ and R^{4a} are absent and R⁵ is hydrogen, the reaction of a compound of the formula (X) with a compound of the formula R⁶NCO under urea forming conditions;
(g) when E is CR⁸R^{8a}, the coupling of a compound of the formula (XVIa) or (XVIb), where A′ is the residue of the group A and R^{x} is hydrogen or a methyl or ethyl group wherein the methyl and ethyl groups are optionally substituted with one or more fluorine atoms, with a compound of the formula (XVII) where Hal is a halogen such as bromine, in the presence of a transition metal catalyst such as a palladium catalyst and/or a copper catalyst:

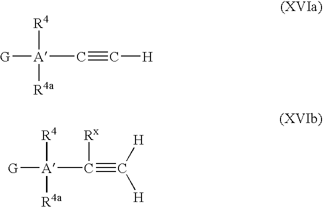

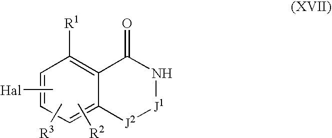

and thereafter subjecting the product of the reaction to reduction, for example catalytic reduction in the presence of a transition metal catalyst such as palladium on charcoal;
(h) when E is O, S or NR⁷, the reaction of a compound of the formula (XVII) or an N-protected derivative thereof, with a compound of the formula (XIII) or (XV) in the presence of a palladium or copper catalyst;
(i) when E is C(R⁸)═C(R⁸), the reaction of a compound of the formula (XVII) with a compound with a compound of the formula (XX):

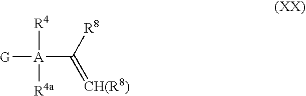

in the presence of a palladium (II) catalyst such as palladium (II) acetate; and
(j) optionally the conversion of one compound of the formula (I) to another compound of the formula (I).

Processes (a) and (b) above are carried out by reacting the amine and carboxylic acid together under conditions suitable for amide bond formation. For example, the coupling reaction can be carried out in the presence of a reagent of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Particular coupling reagents include EDC (EDAC) and DCC in combination with HOAt or HOBt, and EDC in combination with 4-dimethylaminopyridine (DMAP).

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

As an alternative, a reactive derivative of the carboxylic acid, e.g. an anhydride or acid chloride, may be used. Reaction with a reactive derivative such an anhydride or acid chloride is typically accomplished by stirring the amine and acid chloride/anhydride at room temperature in the presence of a base such as pyridine or triethylamine.

Acid chlorides may be prepared by reaction of the carboxylic acid with thionyl chloride, or oxalyl chloride/DMF or by reaction of a carboxylate salt with oxalyl chloride in accordance with know methods.

Amines of the formula (X) are commercially available or can be obtained by methods well known to those skilled in the art of organic chemistry.

Carboxylic acids of the formula (XI) are commercially available or can be prepared by methods well known to the skilled chemist, or the methods described in the experimental section of this application and methods analogous thereto.

Carboxylic acids of the formula (XII) can be prepared by the reaction of a dicarboxylic acid of the formula (XVIII), or a protected derivative thereof, with formamide (to give a compound wherein J²-J¹ is N═C), or with urea (to give a compound wherein J²-J¹ is HN—CO). The reactions are typically carried out at an elevated temperature (e.g. up to about 180° C.).

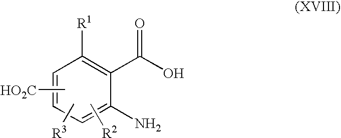

Compounds of the formula (XVIII) are commercially available or can be made by methods well known to those skilled in the art of organic chemistry.

In process (c), an alcohol or thiol, usually in the form of an alkoxide or thiolate anion, is reacted with a compound of the formula (XIV) in which L¹ is a leaving group. One particular leaving group is the present context is fluorine. Similarly, in process (d), an alcohol or thiol, usually in the form of an alkoxide or thiolate anion, is reacted with a compound of the formula (XVa) in which L² is a leaving group.

One particular leaving group in this context is bromine. In both processes, the thiolate or alkoxide anions are typically formed in situ by a base such as a metal hydride, e.g. an alkali metal hydride such as sodium hydride, in an anhydrous polar solvent such as dimethyl formamide. In order to prevent undesirable side reactions involving the quinazolinone N—H group, the amide nitrogen atom of the quinazolinone structure can be protected with a suitable protecting groups (see below for list of protecting groups), one particular protecting group being 2,4-dimethoxybenzyl.

Compounds of the formula (XVII) can be prepared by cyclisation of ortho amino-benzoic acids of the formula (XIX) with either formamide (to give a compound where J²-J¹ is N═C), or with urea (to give a compound wherein J²-J¹ is HN—CO).

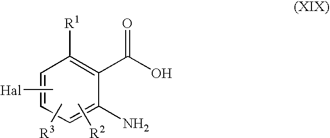

Amino benzoic acids of the formula (XIX) in turn can be prepared from the corresponding ortho-nitrobenzoic acid by reduction with a reducing agent such as Raney nickel/H₂. Substituted ortho-nitrobenzoic acids are commercially available or can be prepared by means of known techniques.

In process (e), an amine compound of the formula (XIII), or protected from thereof, is reacted with a compound of the formula (XIV) or (XVII). The reaction can be carried out in a polar solvent, e.g. an aqueous solvent such as distilled water, at an elevated temperature, for example a temperature up to about 180° C. The heating of the reaction mixture may be effected using a microwave oven, for example.

Alternatively (process (h)), the coupling of a compound of the formula (XVII) with an amine of the formula (XIII) or an alcohol or thiol of the formula (XV) can be achieved by means of a Buchwald-Hartwig type reaction (see Review: J. F. Hartwig, Angew. Chem. Int. Ed. 37, 2046-2067 (1998)) in the presence of a palladium catalyst such as tris-(dibenzylideneacetone)-di-palladium (Pd₂(dba)₃, 2,2′-bis(diphenylphosphino)-1′1-binaphthyl (BINAP) and a strong base such as sodium tert-butoxide.

In process (g), the compound of the formula (XVII), wherein the halogen “Hal” is typically a bromine atom, is reacted with an alkyne of the formula (XVIa) in the presence of palladium (II) (e.g. PdCl₂(PPh₃)₂, copper (I) (e.g. CuI) and a base (e.g. triethylamine). The reaction can be carried out in an anhydrous solvent such as dimethylformamide with moderate heating, for example to a temperature in the range 40-60° C.

The reaction of a compound of the formula (XVII) with an alkene of the formula (XVIb) (process g) or an alkene of the formula (XX) (process (i) can be carried out under conditions known for the Heck reaction or conditions analogous thereto (see for example Advanced Organic Chemistry, by Jerry March, 4^{th} edition, pp 717-718, Wiley Interscience, New York. Thus, for example, the reaction can be carried out in the presence of a palladium catalyst such as palladium (II) acetate and a base such as dicyclohexylmethylamine. The reaction is typically carried out at an elevated temperature (e.g. in excess of 100° C.) in a dry polar solvent such as N-methylpyrrolidinone, and usually under an inert atmosphere.

Alkenes of the formula (XX) can be prepared by a variety of methods well known to the skilled person. For example, compounds of the formula (XX) wherein R⁸ is hydrogen, or compounds of the formula (XVIb) wherein R^{x} is hydrogen, can be prepared from aldehydes of the formula (XXI) by reaction with methyltriphenylphosphonium iodide in the presence of an alkyl lithium such as butyl lithium. The reaction is typically carried out in a polar aprotic solvent such as THF at temperature below 0° C., e.g. −78° C.

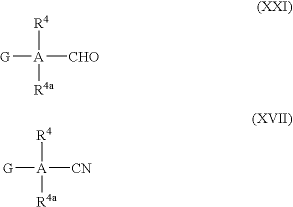

The aldehyde (XXI) can be formed by partial reduction and hydrolysis of a nitrile (XXII). This procedure is preferably carried out using di-isobutyl aluminium hydride in an inert solvent such as toluene or benzene at a low temperature, for example −78° C.

In process (f), a compound of the formula (X) is reacted with an isocyanate R⁶NCO under conditions suitable for forming a urea. The reaction can be carried out in a polar anhydrous solvent such as 1,4-dioxan at an elevated temperature, for example in a sealed tube at a temperature of about 100° C.

Once formed, a compound of the formula (I) can be converted into another compound of the formula (I) by any of a wide range of methods well known to the skilled person.

Examples of functional group interconversions and reagents and conditions for carrying out such conversions can be found in, for example, Advanced Organic Chemistry, by Jerry March, 4^{th} edition, 119, Wiley Interscience, New York, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8).

### Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NR^{c}O—R) or a urethane (—NR^{c}O—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2-(phenylsulphonyl)ethyloxy amide (—NH-Psec). Other protecting groups for amines, such as cyclic amines and heterocyclic N—H groups, include toluenesulphonyl(tosyl) and methanesulphonyl(mesyl) groups and benzyl groups such as a para-methoxybenzyl (PMB) group. A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

### Chemical Intermediates

Many of the chemical intermediates described above are novel and such novel intermediates form a further aspect of the invention.

### Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g. lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

### Protein Kinase Inhibitory Activity

The activity of the compounds of the invention as inhibitors of protein kinase A and/or protein kinase B can be measured using the assays set forth in the examples below and the level of activity exhibited by a given compound can be defined in terms of the IC₅₀ value. Preferred compounds of the present invention are compounds having an IC₅₀ value of less than 1 micromolar, more preferably less than 0.1 micromolar, in particular against protein kinase B.

### Therapeutic Uses

### Prevention or Treatment of Proliferative Disorders

The compounds of the formula (I) are inhibitors of protein kinase A and protein kinase B. As such, they are expected to be useful in providing a means of preventing the growth of or inducing apoptosis of neoplasias. It is therefore anticipated that the compounds will prove useful in treating or preventing proliferative disorders such as cancers. In particular tumours with deletions or inactivating mutations in PTEN or loss of PTEN expression or rearrangements in the (T-cell lytmphocyte) TCL-1 gene may be particularly sensitive to PKB inhibitors. Tumours which have other abnormalities leading to an upregulated PKB pathway signal may also be particularly sensitive to inhibitors of PKB. Examples of such abnormalities include but are not limited to overexpression of one or more PI3K subunits, over-expression of one or more PKB isoforms, or mutations in PI3K, PDK1, or PKB which lead to an increase in the basal activity of the enzyme in question, or upregulation or overexpression or mutational activation of a growth factor receptor such as a growth factor selected from the epidermal growth factor receptor (EGFR), fibroblast growth factor receptor (FGFR), platelet derived growth factor receptor (PDGFR), insulin-like growth factor 1 receptor (IGF-1R) and vascular endothelial growth factor receptor (VEGFR) families.

It is also envisaged that the compounds of the invention will be useful in treating other conditions which result from disorders in proliferation or survival such as viral infections, and neurodegenerative diseases for example. PKB plays an important role in maintaining the survival of immune cells during an immune response and therefore PKB inhibitors could be particularly beneficial in immune disorders including autoimmune conditions.

Therefore, PKB inhibitors could be useful in the treatment of diseases in which there is a disorder of proliferation, apoptosis or differentiation.

PKB inhibitors may also be useful in diseases resulting from insulin resistance and insensitivity, and the disruption of glucose, energy and fat storage such as metabolic disease and obesity.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, esophagus, gall bladder, ovary, pancreas e.g. exocrine pancreatic carcinoma, stomach, cervix, endometrium, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

A further subset of cancers includes breast cancer, ovarian cancer, prostate cancer, endometrial cancer and glioma.

It is also possible that some protein kinase B inhibitors can be used in combination with other anticancer agents. For example, it may be beneficial to combine of an inhibitor that induces apoptosis with another agent which acts via a different mechanism to regulate cell growth thus treating two of the characteristic features of cancer development. Examples of such combinations are set out below.

### Immune Disorders

Immune disorders for which PKA and PKB inhibitors may be beneficial include but are not limited to autoimmune conditions and chronic inflammatory diseases, for example systemic lupus erythematosus, autoimmune mediated glomerulonephritis, rheumatoid arthritis, psoriasis, inflammatory bowel disease, and autoimmune diabetes mellitus, Eczema hypersensitivity reactions, asthma, COPD, rhinitis, and upper respiratory tract disease.

### Other Therapeutic Uses

PKB plays a role in apoptosis, proliferation, differentiation and therefore PKB inhibitors could also be useful in the treatment of the following diseases other than cancer and those associated with immune dysfunction; viral infections, for example herpes virus, pox virus, Epstein-Barr virus, Sindbis virus, adenovirus, HIV, HPV, HCV and HCMV; prevention of AIDS development in HIV-infected individuals; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, degenerative diseases of the musculoskeletal system, for example, osteoporosis and arthritis, aspirin-sensitive rhinosinusitis, cystic fibrosis, multiple sclerosis, kidney diseases.

### Methods of Treatment

It is envisaged that the compounds of the formula (I) will useful in the prophylaxis or treatment of a range of disease states or conditions mediated by protein kinase A and/or protein kinase B. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile manner.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents or treatments that may be administered together (whether concurrently or at different time intervals) with the compounds of the formula (I) include but are not limited to:
Topoisomerase I inhibitors
Antimetabolites
Tubulin targeting agents
DNA binder and topo II inhibitors
Alkylating Agents
Monoclonal Antibodies.
Anti-Hormones
Signal Transduction Inhibitors
Proteasome Inhibitors
DNA methyl transferases
Cytokines and retinoids
Radiotherapy.

For the case of protein kinase A inhibitors or protein kinase B inhibitors combined with other therapies the two or more treatments may be given in individually varying dose schedules and via different routes.

Where the compound of the formula (I) is administered in combination therapy with one or more other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

### Methods of Diagnosis

Prior to administration of a compound of the formula (I), a patient may be screened to determine whether a disease or condition from which the patient is or may be suffering is one which would be susceptible to treatment with a compound having activity against protein kinase A and/or protein kinase B.

For example, a biological sample taken from a patient may be analysed to determine whether a condition or disease, such as cancer, that the patient is or may be suffering from is one which is characterised by a genetic abnormality or abnormal protein expression which leads to up-regulation of PKA and/or PKB or to sensitisation of a pathway to normal PKA and/or PKB activity, or to upregulation of a signal transduction component upstream of PKA and/or PKB such as, in the case of PKB, P13K, GF receptor and PDK 1 & 2.

Alternatively, a biological sample taken from a patient may be analysed for loss of a negative regulator or suppressor of the PKB pathway such as PTEN. In the present context, the term “loss” embraces the deletion of a gene encoding the regulator or suppressor, the truncation of the gene (for example by mutation), the truncation of the transcribed product of the gene, or the inactivation of the transcribed product (e.g. by point mutation) or sequestration by another gene product.

The term up-regulation includes elevated expression or over-expression, including gene amplification (i.e. multiple gene copies) and increased expression by a transcriptional effect, and hyperactivity and activation, including activation by mutations. Thus, the patient may be subjected to a diagnostic test to detect a marker characteristic of up-regulation of PKA and/or PKB. The term diagnosis includes screening. By marker we include genetic markers including, for example, the measurement of DNA composition to identify mutations of PKA and/or PKB. The term marker also includes markers which are characteristic of up regulation of PKA and/or PKB, including enzyme activity, enzyme levels, enzyme state (e.g. phosphorylated or not) and mRNA levels of the aforementioned proteins.

The above diagnostic tests and screens are typically conducted on a biological sample selected from tumour biopsy samples, blood samples (isolation and enrichment of shed tumour cells), stool biopsies, sputum, chromosome analysis, pleural fluid, peritoneal fluid, or urine.

Identification of an individual carrying a mutation in PKA and/or PKB or a rearrangement of TCL-1 or loss of PTEN expression may mean that the patient would be particularly suitable for treatment with a PKA and/or PKB inhibitor. Tumours may preferentially be screened for presence of a PKA and/or PKB variant prior to treatment. The screening process will typically involve direct sequencing, oligonucleotide microarray analysis, or a mutant specific antibody.

Methods of identification and analysis of mutations and up-regulation of proteins are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR.

Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of upregulation of PKB, or detection of PKB variants could be applicable in the present case.

Therefore all of these techniques could also be used to identify tumours particularly suitable for treatment with PKA and/or PKB inhibitors.

For example, as stated above, PKB beta has been found to be upregulated in 10-40% of ovarian and pancreatic cancers (Bellacosa et al 1995, Int. J. Cancer 64, 280-285; Cheng et al 1996, PNAS 93, 3636-3641; Yuan et al 2000, Oncogene 19, 2324-2330). Therefore it is envisaged that PKB inhibitors, and in particular inhibitors of PKB beta, may be used to treat ovarian and pancreatic cancers.

PKB alpha is amplified in human gastric, prostate and breast cancer (Staal 1987, PNAS 84, 5034-5037; Sun et al 2001, Am. J. Pathol. 159, 431-437). Therefore it is envisaged that PKB inhibitors, and in particular inhibitors of PKB alpha, may be used to treat human gastric, prostate and breast cancer.

Increased PKB gamma activity has been observed in steroid independent breast and prostate cell lines (Nakatani et al 1999, J. Biol. Chem. 274, 21528-21532). Therefore it is envisaged that PKB inhibitors, and in particular inhibitors of PKB gamma, may be used to treat steroid independent breast and prostate cancers.

### EXPERIMENTAL

The invention will now be illustrated, but not limited, by reference to the specific embodiments described in the following procedures and examples.

The starting materials for each of the procedures described below are commercially available unless otherwise specified.

Proton magnetic resonance (¹H NMR) spectra were recorded on a Bruker AV400 instrument operating at 400.13 MHz, in Me-d₃-OD at 27° C., unless otherwise stated and are reported as follows: chemical shift δ/ppm (number of protons, multiplicity where s=singlet, d=doublet, t=triplet, q=quartet, m=multiplet, br=broad). The residual protic solvent MeOH (δ_{H}=3.31 ppm) was used as the internal reference.

In the examples, the compounds prepared were characterised by liquid chromatography and mass spectroscopy using the system and operating conditions set out below. Where chlorine is present, the mass quoted for the compound is for ³⁵Cl. Where bromide is present the mass quoted for the compound is ⁷⁹Br. The two systems were equipped with identical chromatography columns and were set up to run under the same operating conditions. The operating conditions used are also described below.

Platform System
HPLC System: Waters 2795
Mass Spec Detector: Micromass Platform LC
PDA Detector: Waters 2996 PDA
Acidic Analytical conditions 1:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ Hydro-RP 80 A, 2.0 × 50 mm |

Acidic Analytical conditions 2:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ MAX-RP 80 A, 2.0 × 50 mm |

Acidic Extended run conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 80 A, 2.0 × 150 mm |

Basic Analytical conditions 1:

| | |
|---|---|
| Eluent A: | H₂O (10 mM NH₄HCO₃ buffer adjusted to pH = 9.5 with NH₄OH) |
| Eluent B: | CH₃CN |
| Gradient: | 05-95% eluent B over 3.5 minutes |
| Flow: | 0.8 ml/min |
| Column: | Thermo Hypersil-Keystone BetaBasic-18 5 μm 2.1 × 50 mm |

Basic Analytical conditions 2:

| | |
|---|---|
| Eluent A: | H₂O (10 mM NH₄HCO₃ buffer adjusted to pH = 9.5 with NH₄OH) |
| Eluent B: | CH₃CN |
| Gradient: | 05-95% eluent B over 3.5 minutes |
| Flow: | 0.8 ml/min |
| Column: | Phenomenex Luna C18(2) 5 μm 2.0 × 50 mm |

Basic Analytical conditions 3:

| | |
|---|---|
| Eluent A: | H₂O (10 mM NH₄HCO₃ buffer adjusted to pH = 9.2 with NH₄OH) |
| Eluent B: | CH₃CN |
| Gradient: | 05-95% eluent B over 3.5 minutes |
| Flow: | 0.8 ml/min |
| Column: | Phenomenex Luna C18(2) 5 μm 2.0 × 50 mm |

Basic Analytical conditions 4:

| | |
|---|---|
| Eluent A: | H₂O (10 mM NH₄HCO₃ buffer adjusted to pH = 9.2 with NH₄OH) |
| Eluent B: | CH₃CN |
| Gradient: | 05-95% eluent B over 3.5 minutes |
| Flow: | 0.8 ml/min |
| Column: | Phenomenex Gemini 5μ 2.0 × 50 mm |

Basic Extended run conditions 1:

| | |
|---|---|
| Eluent A: | H₂O (10 mM NH₄HCO₃ buffer adjusted to pH = 9.2 with NH₄OH) |
| Eluent B: | CH₃CN |
| Gradient: | 05-95% eluent B over 15 minutes |
| Flow: | 0.8 ml/min |
| Column: | Phenomenex Luna C18(2) 5 μm 2.0 × 50 mm |

Basic Extended run conditions 2:

| | |
|---|---|
| Eluent A: | H₂O (10 mM NH₄HCO₃ buffer adjusted to pH = 9.2 with NH₄OH) |
| Eluent B: | CH₃CN |
| Gradient: | 05-95% eluent B over 15 minutes |
| Flow: | 0.8 ml/min |
| Column: | Phenomenex Luna C18(2) 5μ 2.0 × 50 mm |

Polar Analytical conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Phenomenex Synergi 4μ MAX-RP 80 A, 2.0 × 50 mm |

MS conditions:

| | |
|---|---|
| Capillary voltage: | 3.6 kV |
| Cone voltage: | 30 V |
| Source Temperature: | 120° C. |
| Scan Range: | 165-700 amu |
| Ionisation Mode: | ElectroSpray Negative, Positive or Positive & |
| | Negative |

Agilent System
HPLC System: Agilent 1100 series
Mass Spec Detector Agilent LC/MSD VL
Multi Wavelength Detector: Agilent 1100 series MWD
Software: HP Chemstation
Chiral Analytical conditions:

| | |
|---|---|
| Eluent: | methanol + 0.4% acetic acid + 0.1% triethylamine |
| | at room temperature |
| Flow: | 2.0 ml/min |
| Total time: | 13 min |
| Inj. Volume: | 10 μL |
| Sample Conc: | 2 mg/ml |
| Column: | Astec, Chirobiotic V2; 250 × 4.6 mm |

Chiral Preparative conditions:

| | |
|---|---|
| Eluent: | methanol + 0.4% acetic acid + 0.1% triethylamine |
| | at room temperature |
| Flow: | 6.0 ml/min |
| Total time: | 21 min |
| Inj. Volume: | 100 μL |
| Sample Conc: | 20 mg/ml |
| Column: | Astec, Chirobiotic V2; 250 × 10 mm |

MS conditions (just analytical method):

| | |
|---|---|
| Capillary voltage: | 3000 V |
| Fragmentor: | 150 |
| Gain: | 1.00 |
| Drying gas: | 12.0 L/min |
| Drying gas T: | 350° C. |
| Nebulizer pressure: | 35 (psig) |
| Scan Range: | 125-800 amu |
| Ionisation Mode: | ElectroSpray Positive |

LCT System 1
HPLC System: Waters Alliance 2795 Separations Module
Mass Spec Detector: Waters/Micromass LCT
UV Detector: Waters 2487 Dual λ Absorbance Detector
Polar Analytical conditions:

| Eluent A: | Methanol | |
|---|---|---|
| Eluent B: | 0.1% Formic Acid in Water | |
| Gradient: | | |
| Time (mins) | A | B |
| 0 | 10 | 90 |
| 0.5 | 10 | 90 |
| 6.5 | 90 | 10 |
| 10 | 90 | 10 |
| 10.5 | 10 | 90 |
| 15 | 10 | 90 |

| | |
|---|---|
| Flow: | 1.0 ml/min |
| Column: | Supelco DISCOVERY C₁₈ 5 cm × 4.6 mm |
| | i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

LCT System 2
HPLC System: Waters Alliance 2795 Separations Module
Mass Spec Detector: Waters/Micromass LCT
UV Detector: Waters 2487 Dual λ Absorbance Detector
Analytical conditions:

| Eluent A: | Methanol | |
|---|---|---|
| Eluent B: | 0.1% Formic Acid in Water | |
| Gradient: | | |
| Time (mins) | A | B |
| 0 | 10 | 90 |
| 0.6 | 10 | 90 |
| 1.0 | 20 | 80 |
| 7.5 | 90 | 10 |
| 9 | 90 | 10 |
| 9.5 | 10 | 90 |
| 10 | 10 | 90 |

| | |
|---|---|
| Flow: | 1 ml/min |
| Column: | Supelco DISCOVERY C₁₈ 5 cm × 4.6 mm |
| | i.d., 5 μm |

MS conditions:

| | |
|---|---|
| Capillary voltage: | 3500 v (+ve ESI), 3000 v (−ve ESI) |
| Cone voltage: | 40 v (+ve ESI), 50 v (−ve ESI) |
| Source Temperature: | 100° C. |
| Scan Range: | 50-1000 amu |
| Ionisation Mode: | +ve/−ve electrospray ESI (Lockspray ™) |

In the examples below, the following key is used to identify the LCMS conditions used:

| | |
|---|---|
| PS-A1 | Platform System - acidic analytical conditions 1 |
| PS-A2 | Platform System - acidic analytical conditions 2 |
| PS-AE | Platform System - acidic extended run analytical conditions |
| PS-B1 | Platform System - basic analytical conditions 1 |
| PS-B2 | Platform System - basic analytical conditions 2 |
| PS-B3 | Platform System - basic analytical conditions 3 |
| PS-B4 | Platform System - basic analytical conditions 4 |
| PS-BE1 | Platform System - basic extended run analytical conditions 1 |
| PS-BE2 | Platform System - basic extended run analytical conditions 1 |
| PS-P | Platform System - polar analytical conditions |
| AG-CA | Agilent System - chiral analytical conditions |
| AG-CP | Agilent System - chiral preparative conditions |
| LCT1 | LCT System 1 - polar analytical conditions |
| LCT2 | LCT System 2 - polar analytical conditions |

### Example 1

### 4-Amino-2-(3,4-dichloro-phenyl)-N-(4-oxo-3,4-dihydro-quinazolin-7-yl)-butyramide

### 1A. [3-(3,4-Dichloro-phenyl)-3-(4-oxo-3,4-dihydro-quinazolin-7-ylcarbamoyl)-propyl]-carbamic acid tert-butyl ester

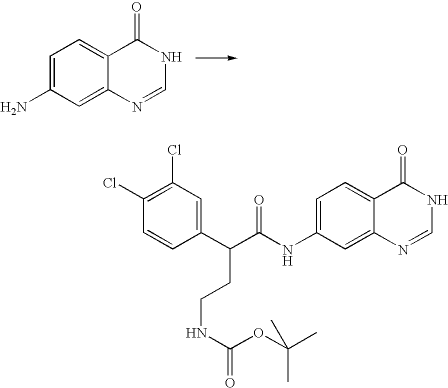

To a reaction vial was added 7-amino-3H-quinazolin-4-one (0.459 g, 2.85 mmol) (SPECS, 907/25004783), 4-tert-butoxycarbonylamino-2-(3,4-dichloro-phenyl)-butyric acid* (1 g, 2.87 mmol), 1-(3-dimethylaminopropyl)-3-ethylcarbodiimide hydrochloride (0.273 g, 1.42 mmol) and a crystal of 4-(dimethylamino)pyridine. Anhydrous N,N-dimethylformamide (4.6 ml) was added and the reaction mixture was sealed and heated at 55° C. with stirring for 16 hours. Solvent was removed under reduced pressure and the residue was dissolved in DCM. The organic layer was washed with water, saturated sodium bicarbonate solution then dried (MgSO₄) and solvent was removed under reduced pressure. The residue was purified using flash silica chromatography eluting with methanol/ethyl acetate (4:96) to afford the title compound as a yellow gum (0.077 g, 11% yield). LC/MS: (PS-B1) Rₜ 2.95 [M+H]⁺ 491.06.
This starting material was made by the method described in international patent application WO 03/064397 A1

### 1B. 4-Amino-2-(3,4-dichloro-phenyl)-N-(4-oxo-3,4-dihydro-quinazolin-7-yl)butyramide

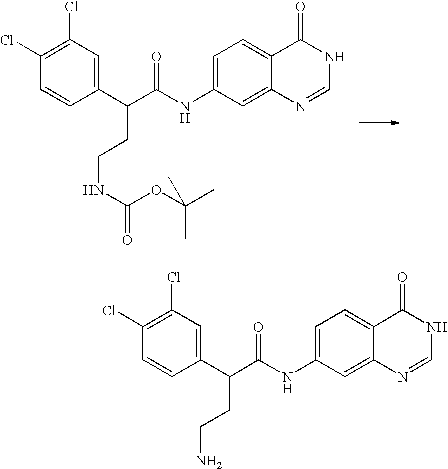

[3-(3,4-Dichloro-phenyl)-3-(4-oxo-3,4-dihydro-quinazolin-7-ylcarbamoyl)propyl]-carbamic acid tert-butyl ester (0.106 g, 0.215 mmol) was dissolved in dichloromethane (8 ml). To this solution was added 4N HCl in 1,4-dioxane (0.537 ml, 2.15 mmol). The reaction mixture was stirred for 2 hours then solvent was removed under reduced pressure. The residue was purified first by ion exchange chromatography and then by flash silica chromatography, eluting with 2N ammonia in methanol/dichloromethane (20/80) to afford the title compound as a white solid (0.040 g, 48% yield). LC/MS: (PS-B1) Rₜ 2.48 [M+H]⁺ 390.96. ¹H NMR (Me-d₃-OD) [symbol] 1.80-1.91 (1H, m), 2.15-2.25 (1H, m), 2.49-2.63 (2H, m), 3.68 (1H, t), 7.27 (1H, d), 7.39 (1H, d), 7.51-7.57 (2H, m), 7.97 (1H, s), 7.99 (1H, s), 8.03 (1H, d).

### Example 2

### 2-(4-Chloro-phenyl)-4-methylamino-N-(4-oxo-3,4-dihydro-quinazolin-7-yl)-butyramide

### 2A. [3-(4-Chloro-phenyl)-3-cyano-propyl]-methyl-carbamic acid tert-butyl ester

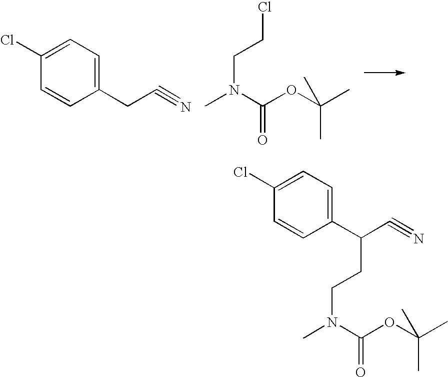

[3-(4-Chloro-phenyl)-3-cyano-propyl]-methyl-carbamic acid tert-butyl ester was made using a method described in U.S. Pat. No. 4,783,537. The starting material (2-Chloro-ethyl)-methyl-carbamic acid tert-butyl ester was made using a method described in J. Med. Chem. 1998, 41, 5429-5444.

### 2B. Sodium 4-(tert-butoxycarbonyl-methyl-amino)-2-(4-chloro-phenyl)-butyrate

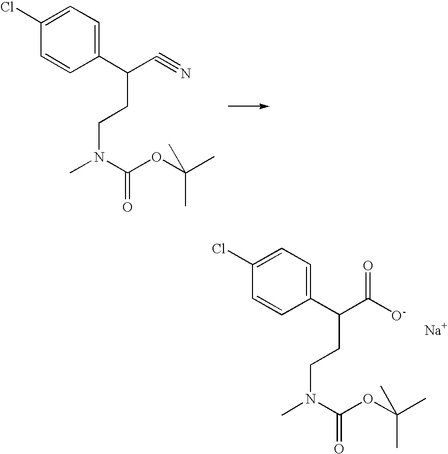

Sodium 4-(tert-butoxycarbonyl-methyl-amino)-2-(4-chloro-phenyl)-butyrate was made using a method described in J. Med. Chem. 1989, Vol. 32, No. 4, 793-799.

### 2C. [3-(4-Chloro-phenyl)-3-(4-oxo-3,4-dihydro-quinazolin-7-ylcarbamoyl)-propyl]-methyl-carbamic acid tert-butyl ester

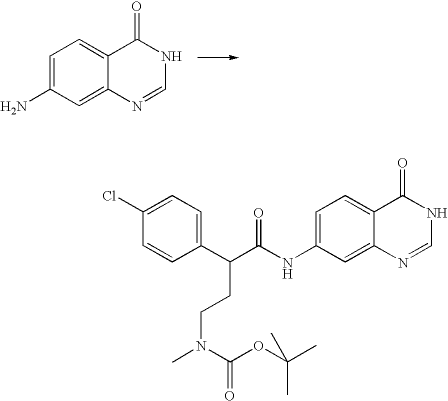

7-Amino-3H-quinazolin-4-one (0.3 g, 1.86 mmol) was reacted with sodium 4-(tert-butoxycarbonyl-methyl-amino)-2-(4-chloro-phenyl)-butyrate (0.356 g, 1.86 mmol) following the procedure set out in Example 3D. For work-up the reaction mixture was diluted with ethyl acetate and washed with saturated sodium bicarbonate solution. The aqueous was extracted twice more with ethyl acetate. The organics were combined, dried (MgSO₄) and solvent was removed under reduced pressure. The residue was purified by flash silica chromatography, eluting with methanol/ethyl acetate (4:96) to yield the title compound as a colourless gum (0.039 g, 4% yield). LC/MS: (PS-B2) Rₜ 3.03 [M+H]⁺ 471.19.

### 2D. 2-(4-Chloro-phenyl)-4-methylamino-N-(4-oxo-3,4-dihydro-quinazolin-7-yl)-butyramide

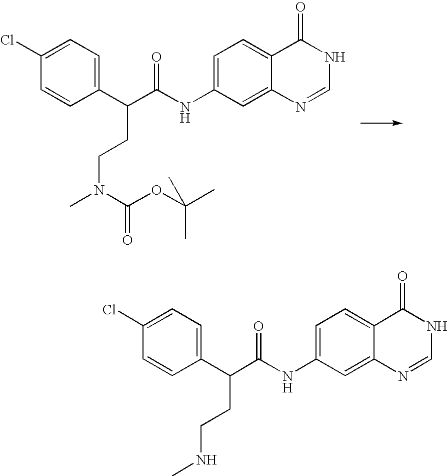

[3-(4-Chloro-phenyl)-3-(4-oxo-3,4-dihydro-quinazolin-7-ylcarbamoyl)-propyl]-methyl-carbamic acid tert-butyl ester (0.039 g, 0.083 mmol) was converted to 2-(4-Chloro-phenyl)-4-methylamino-N-(4-oxo-3,4-dihydro-quinazolin-7-yl)-butyramide using the same procedure as described in Example 1B except that saturated HCl in ethyl acetate (3 ml) was used instead of 4N HCl in 1,4-dioxane. The title compound was afforded as a yellow solid (0.011 g, 35% yield). LC/MS: (PS-B2) Rₜ 2.38 [M+H]⁺ 371.10. ¹H NMR (Me-d₃-OD) [symbol] 1.90-1.99 (1H, m), 2.23-2.33 (1H, m), 2.40 (3H, s), 2.55-2.71 (2H, m), 3.70 (1H, t), 7.27 (2H, d), 7.34 (2H, d), 7.55 (1H, d), 7.96 (1H, s), 8.00-8.05 (2H, m).

### Example 3

### 4-Oxo-3,4-dihydro-quinazoline-7-carboxylic acid[3-amino-1-(4-chloro-phenyl-propyl]-amide hydrochloride

### 3A. 4-Oxo-3,4-dihydro-quinazoline-7-carboxylic acid

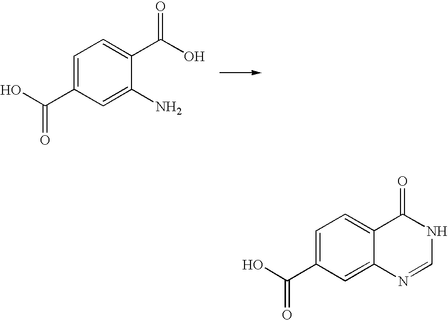

2-Amino-terephthalic acid (4.5 g, 24.8 mmol) was suspended in formamide (30 ml). The mixture was heated at 180° C. with stirring for 1 hour. The reaction mixture was then allowed to cool to room temperature and stand for 16 hours. A precipitate had formed upon standing. The precipitate was filtered off, washing through with acetone to yield the title compound as a white solid, (1.25 g, 27% yield). LC/MS: (PS-A2) Rₜ 1.45 [M+H]⁺ 190.92.

### 3B. [3-tert-Butoxycarbonylamino-3-(4-chloro-phenyl)-propyl]-carbamic acid benzyl ester

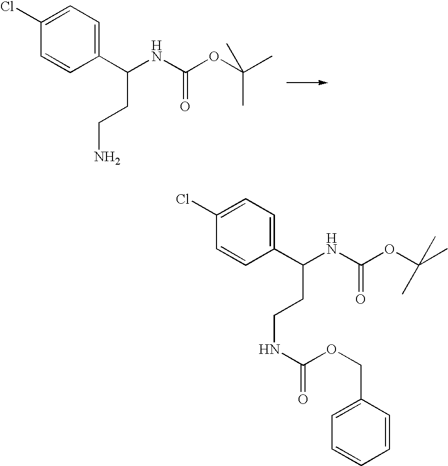

[3-Amino-1-(4-chloro-phenyl)-propyl]-carbamic acid tert-butyl ester (0.742 g, 2.61 mmol) (Pharmacore, 550213) was suspended in dichloromethane (11.1 ml) and N-ethyl-diisopropylamine (0.5 ml, 2.87 mmol) was added. The reaction mixture was cooled to 0° C. and benzyl chloroformate (0.41 ml, 2.87 mmol) was added dropwise with stirring. The reaction mixture was then stirred at room temperature for 16 hours. The reaction mixture was diluted with DCM and washed with water. The aqueous was separated and extracted with DCM. The organics were combined, washed with brine, dried (MgSO₄) and solvent was removed under reduced pressure. The residue was purified by flash silica chromatography, eluting with ethyl acetate/petroleum ether (30:70) to yield the title compound as a colourless oil (0.538 g, 49% yield). LC/MS: (PS-A2) Rₜ 3.55 [M+H]⁺ 418.99.

### 3C. [3-Amino-3-(4-chloro-phenyl)-propyl]-carbamic acid benzyl ester

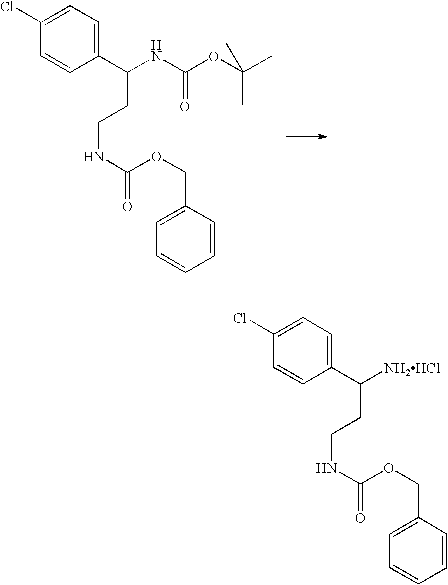

By following the procedure set out in Example 1B but using [3-tert-butoxycarbonylamino-3-(4-chloro-phenyl)-propyl]-carbamic acid benzyl ester (0.538 g, 1.28 mmol) and without the need for purification, the title compound was obtained as the HCl salt (0.436 g, 96% yield). LC/MS: (PS-P)Rₜ 2.59 [M+H]⁺ 318.91.

### 3D. {3-[(4-Chloro-phenyl)-3-(4-oxo-3,4-dihydro-quinazoline-7-carbonyl)amino]-propyl}-carbamic acid benzyl ester

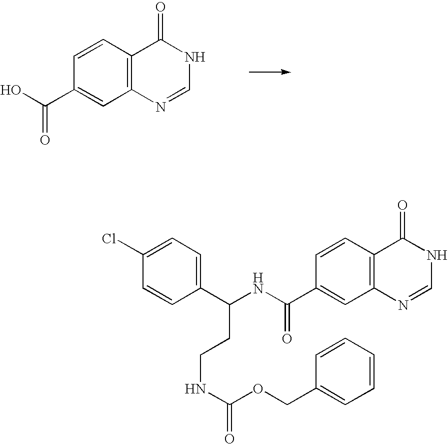

To a reaction vial was added 4-oxo-3,4-dihydro-quinazoline-7-carboxylic acid (0.1 g, 0.526 mmol), [3-amino-3-(4-chloro-phenyl)-propyl]-carbamic acid benzyl ester (0.187 g, 0.526 mmol) and 1-hydroxybenzotriazole (0.071 g, 0.526 mmol). The mixture was suspended in N,N-dimethylformamide (1.53 ml). N-ethyl-diisopropylamine (0.183 ml, 1.052 mmol) was added and the reaction mixture was stirred for 10 minutes at room temperature. 1-(3-Dimethylaminopropyl)-3-ethylcarbodiimide hydrochloride (0.101 g, 0.526 mmol) was then added. The reaction was sealed and heated at 50° C. for 16 hours. The reaction mixture was diluted with water and extracted three times with ethyl acetate. The organics were combined, dried (MgSO₄) and solvent was removed under reduced pressure. The residue was purified by flash silica chromatography, eluting with a gradient of methanol/dichloromethane (2:98 to 5:95) to yield the title compound as a colourless gum (0.138 g, 53% yield). LC/MS: (PS-B2) Rₜ 2.84 [M+H]⁺ 491.06.

### 3E. 4-Oxo-3,4-dihydro-quinazoline-7-carboxylic acid[3-amino-1-(4-chloro-phenyl)-propyl]-amide hydrochloride

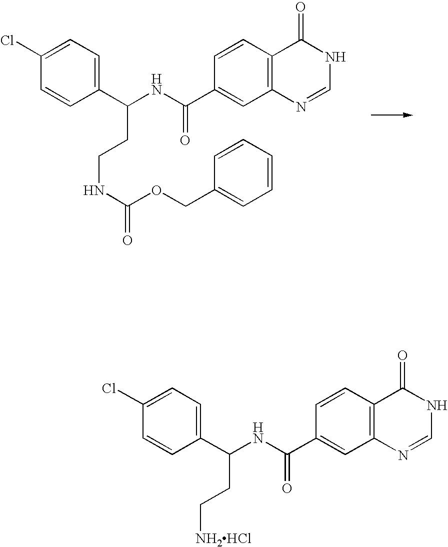

{3-(4-Chloro-phenyl)-3-[(4-oxo-3,4-dihydro-quinazoline-7-carbonyl)-amino]-propyl}-carbamic acid benzyl ester (0.14 g, 0.285 mmol) was dissolved in a solution of 45% HBr in acetic acid (4 ml). After stirring for 30 minutes at room temperature the solvent was removed under reduced pressure. The residue was purified by ion exchange chromatography followed by flash silica chromatography, eluting with a gradient of methanol/dichloromethane (10:90 to 30:70). The product (0.0583 g, 0.163 mmol) was dissolved in dichloromethane (8.39 ml), treated with 4N HCl in 1,4-dioxane (0.408 ml, 1.63 mmol) and stirred at room temperature for 2 hours. The solvent was removed under reduced pressure and the residue was triturated with diethyl ether then filtered to yield the title compound as a white solid (0.05 g, 45% yield). LC/MS: (PS-B2) Rₜ 2.18 [M+H]⁺ 357.02. ¹H NMR (Me-d₃-OD) [symbol] 2.24-2.46 (2H, m), 2.94-3.03 (1H, m), 3.08-3.17 (1H, m), 5.25-5.31 (1H, m), 7.43 (2H, d), 7.51 (2H, d), 8.11 (1H, d), 8.20 (1H, s), 8.39 (1H, d), 8.88 (1H, s).

### Example 4

### 4-Phenyl-piperidine-4-carboxylic acid (4-oxo-3,4-dihydro-quinazolin-7-yl)-amide

### 4A. 4-Phenyl-piperidine-1,4-dicarboxylic acid mono-(9H-fluoren-9-ylmethyl)ester

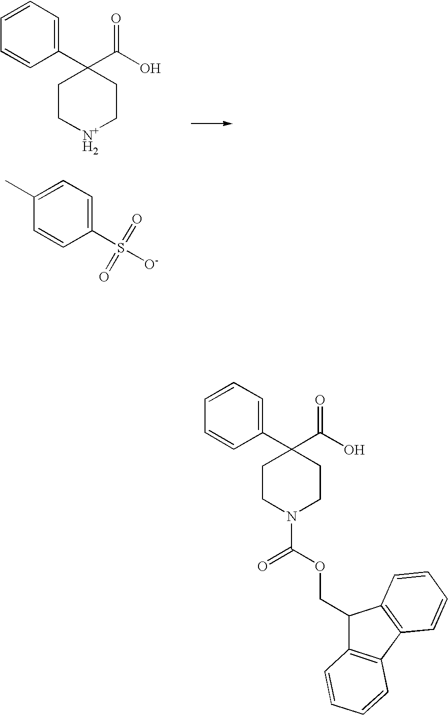

4-Carboxy-4-phenyl-piperidinium toluene-4-sulphonate (5 g, 13.25 mmol) was dissolved in a 1.15N aqueous solution of sodium hydroxide (23 ml). To this was added a solution of carbonic acid 2,5-dioxo-pyrrolidin-1-yl ester 9H-fluoren-9-ylmethyl ester (4.92 g, 14.58 mmol) in 1,4-dioxane (23 ml). The reaction mixture was stirred at room temperature for 72 hours. The resultant suspension was diluted with 2N HCl (aq) and ethyl acetate and then filtered to yield the title compound as a white solid (3.19 g, 56% yield). LC/MS: (PS-A2) Rₜ 3.46 [M+H]⁺ 428.18.

### 4B. 4-Chlorocarbonyl-4-phenyl-piperidine-1-carboxylic acid 9H-fluoren-9-yl methyl ester

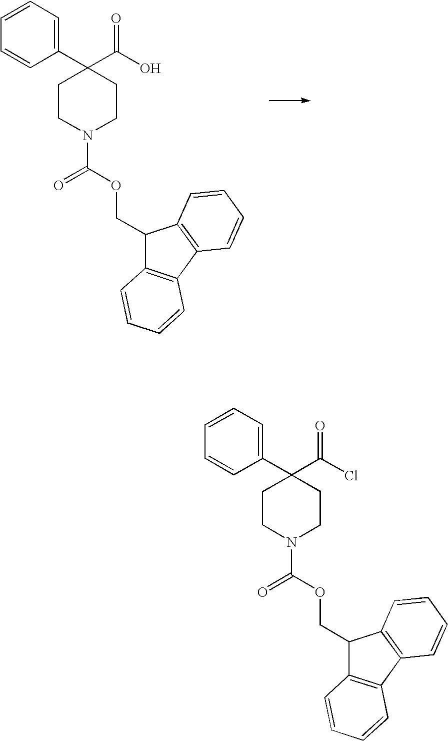

4-Phenyl-piperidine-1,4-dicarboxylic acid mono-(9H-fluoren-9-ylmethyl)ester (1 g, 2.34 mmol) was dissolved in thionyl chloride (20 ml). The resultant solution was heated at 80° C. for 16 hours. The thionyl chloride was removed under reduced pressure and the residue was azeotroped twice with dichloromethane to yield the title compound as a yellow oil (1.1 g, >100% yield). The product was used without further purification. LC/MS (in methanol): (PS-A2) Rₜ 3.88 [M+H]⁺ 442.16 (methyl ester).

### 4C. 4-(4-Oxo-3,4-dihydro-quinazolin-7-ylcarbamoyl)-4-phenyl-piperidine-1-carboxylic acid 9H-fluoren-9-ylmethyl ester

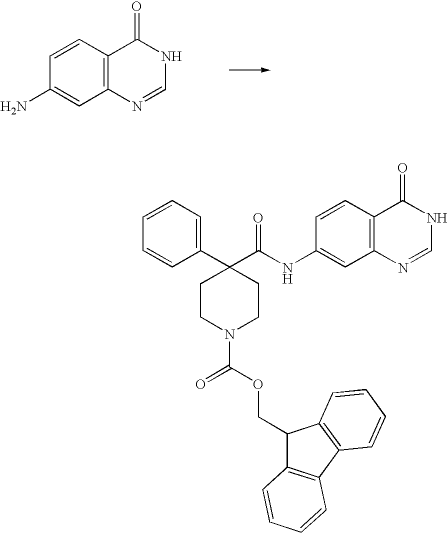

7-Amino-3H-quinazolin-4-one (0.175 g, 1.09 mmol) was suspended in anhydrous dichloromethane (2 ml). Triethylamine (0.163 ml, 1.2 mmol) was added with stirring and the solution was cooled to 0° C. To this solution was added dropwise a solution of 4-chlorocarbonyl-4-phenyl-piperidine-1-carboxylic acid 9H-fluoren-9-yl methyl ester (0.533 g, 1.2 mmol) in dichloromethane (2 ml). The reaction mixture was stirred at room temperature for 15 minutes and then heated at 100° C. in the microwave with stirring for 15 minutes. The reaction mixture was diluted with dichloromethane and washed with 1N HCl. The aqueous was extracted twice more with dichloromethane. The organics were combined, dried (MgSO₄) and solvent was removed under reduced pressure. The residue was purified by flash silica chromatography, eluting with methanol/ethyl acetate (5:95) to yield the title product as a white solid (0.332 g, 35% yield). LC/MS: (PS-B2) Rₜ 3.30 [M+H]⁺ 571.21.

### 4D. 4-Phenyl-piperidine-4-carboxylic acid (4-oxo-3,4-dihydro-quinazolin-7-yl)-amide

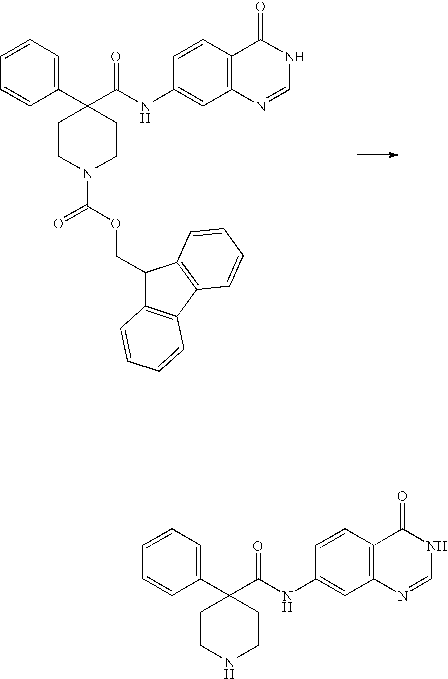

4-(4-Oxo-3,4-dihydro-quinazolin-7-ylcarbamoyl)-4-phenyl-piperidine-1-carboxylic acid 9H-fluoren-9-ylmethyl ester (0.332 g, 0.58 mmol) was dissolved in anhydrous tetrahydrofuran (16.5 ml) and N-(2-mercaptoethyl)aminomethyl polystyrene (3.15 g, 6.3 mmol) was added. The reaction mixture was stirred slowly and 1,8-diazabicyclo[5.4.0]undec-7-ene (0.043 ml, 0.29 mmol) was added. The reaction mixture was stirred at room temperature for 31 hours and the resin was filtered off, washing with tetrahydrofuran followed by methanol. The filtrate was evaporated under reduced pressure and the residue was purified by ion exchange chromatography followed by flash silica chromatography, eluting with 2N ammonia in methanol/dichloromethane (20:80). The product was further purified by preparative liquid chromatography to yield the title compound as a glassy, colourless solid (0.07 g, 35% yield). LC/MS: (PS-B2) Rₜ 2.13 [M+H]⁺ 349.12. ¹H NMR (Me-d₃-OD) [symbol] 2.25-2.36 (2H, m), 2.80-2.88 (2H, m), 3.26-3.45 (4H, m), 7.33-7.37 (1H, m), 7.43-7.53 (4H, m), 7.66 (1H, d), 8.08 (1H, s), 8.10-8.15 (2H, m).

### Example 5

### 1-(4-Chloro-benzyl)-3-(4-oxo-3,4-dihydro-quinazolin-7-yl)-urea

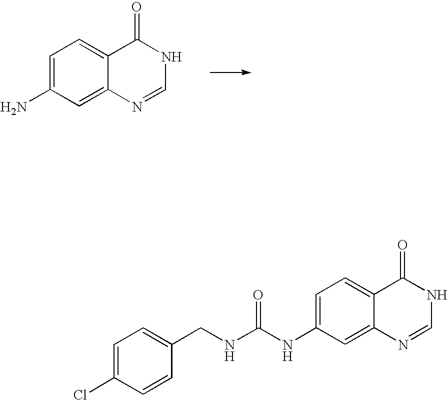

7-Amino-3H-quinazolin-4-one (0.1 g, 0.62 mmol) was reacted with 4-chlorobenzyl isocyanate (0.09 ml, 0.683 mmol) in 1,4-dioxane (1.5 ml) following the procedure set out in Example 13. Following ion exchange chromatography the product was further purified by flash silica chromatography, eluting with methanol:dichloromethane (10:90). The product was triturated in hot methanol, filtered then washed with methanol to yield the title product as a white solid (0.045 g, 22% yield). LC/MS: (PS-B2) Rₜ 2.41 [M+H]⁺ 328.95. ¹H NMR (d₆-DMSO) [symbol] 4.29-4.36 (2H, m), 6.89 (1H, br s), 7.31-7.47 (5H, m), 7.82 (1H, s), 7.94-8.02 (2H, m), 9.16 (1H, br s), 11.94 (1H, br s).

### Example 6

### 1-(4-Fluoro-benzyl)-3-(4-oxo-3,4-dihydro-quinazolin-7-yl)-urea

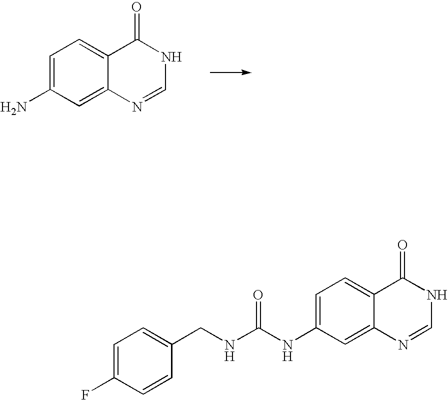

7-Amino-3H-quinazolin-4-one (0.2 g, 1.24 mmol) was reacted with 4-fluorobenzyl isocyanate (0.158 ml, 1.24 mmol) in 1,4-dioxane (3 ml) following the procedure set out in Example 13A. Following ion exchange chromatography, the product crystallized from solution and was filtered then washed with methanol to yield the title compound as white crystalline solid (0.11 g, 28% yield). LC/MS: (PS-B2) Rₜ=2.26 [M+H]⁺ 312.98. ¹H NMR (d₆-DMSO) [symbol] 4.32 (2H, d), 6.85 (1H, t), 7.13-7.20 (2H, m), 7.34-7.39 (2H, m), 7.44 (1H, d), 7.82 (1H, d), 7.97 (1H, d), 8.00 (1H, s), 9.12 (1H, br s), 11.94 (1H, br s).

### Example 7

### 7-(4-Phenyl-piperidin-4-ylmethoxy)-3H-quinazolin-4-one 7A. 7-Fluoro-3H-quinazolin-4-one

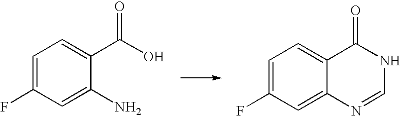

2-Amino-4-fluoro benzoic acid (0.5 g, 3.22 mmol) was suspended in formamide (2 ml) and heated in a CEM Explorer™ microwave at 150° C. with stirring for 15 minutes using 60 Watts of power. Upon cooling to room temperature, a solid precipitated out of solution. The solid was filtered, washing with acetone and then diethyl ether to yield the title compound as a pale grey solid (0.25 g, 47% yield). LC/MS: (PS-A2) Rₜ 1.87 [M+H]⁺ 164.95.

### 7B. (4-Phenyl-piperidin-4-yl)-methanol

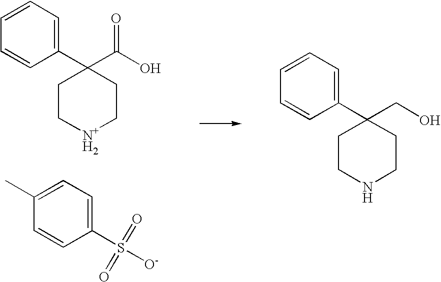

4-Carboxy-4-phenyl-piperidinium toluene-4-sulphonate (0.5 g, 1.32 mmol) was mixed with powdered lithium aluminium hydride (0.139 g, 3.66 mmol) and anhydrous diethyl ether was added (3 ml), pre-cooled to 0° C. The mixture was then cooled to 0° C. with stirring and a suspension of aluminium trichloride (0.417 g, 3.13 mmol) in diethyl ether (3 ml), pre-cooled to 0° C. was added. After addition, the reaction mixture was stirred at room temperature for 18 hours. Water (0.5 ml) was slowly added followed by 2N aqueous sodium hydroxide (0.5 ml). The reaction mixture was filtered through Celite®, washing through with methanol. Solvent was removed under reduced pressure and the residue was purified by ion exchange chromatography followed by flash silica chromatography eluting with a gradient of 2N ammonia in methanol/dichloromethane (20:80 to 30:70 to 40:60). The title compound was afforded as a colourless gum (0.1 g, 40% yield). LC/MS: (PS-B2) Rₜ 1.50 [M+H]⁺ 191.97.

### 7C. 7-(4-Phenyl-piperidin-4-ylmethoxy)-3H-quinazolin-4-one

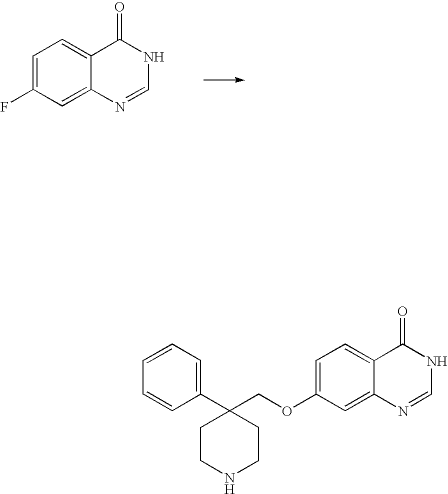

7-Fluoro-3H-quinazolin-4-one (0.0215 g, 0.131 mmol) was reacted with (4-phenyl-piperidin-4-yl)-methanol (0.1 g, 0.523 mmol) using the same procedure as described in Example 12 except that after work up (using ethyl acetate and water) the product was dissolved in methanol (5 ml) and potassium hydroxide (0.115 g, 2.02 mmol) was added as a solid followed by 2 drops of water. The solution was heated at 70° C. with stirring for 1 hour and then more potassium hydroxide was added (0.12 g, 2.11 mmol). Stirring and heating was continued for a further 2 hours. The reaction mixture was cooled to room temperature and then solvent was removed under reduced pressure. The residue was diluted with water and extracted three times with ethyl acetate. The organics were dried (MgSO₄) and solvent was removed under reduced pressure. The residue was purified by ion exchange chromatography followed by flash silica chromatography eluting with a gradient of 2N ammonia in methanol/dichloromethane (20:80 to 30:70) to yield the title compound as a colourless gum (0.0198 g, 45% yield). LC/MS: (PS-B2) Rₜ 2.16 [M+H]⁺ 336.01. ¹H NMR (Me-d₃-OD) 8. 2.08-2.17 (2H, m), 2.37-2.45 (2H, m), 2.73-2.81 (2H, m), 2.98-3.04 (2H, m), 4.07 (2H, s), 7.00-7.06 (2H, m), 7.23-7.28 (1H, m), 7.37-7.43 (2H, m), 7.52-7.56 (2H, m), 8.05-8.09 (2H, m).

### Example 8

### 1-Benzyl-3-(4-oxo-3,4-dihydro-quinazolin-7-yl)-urea

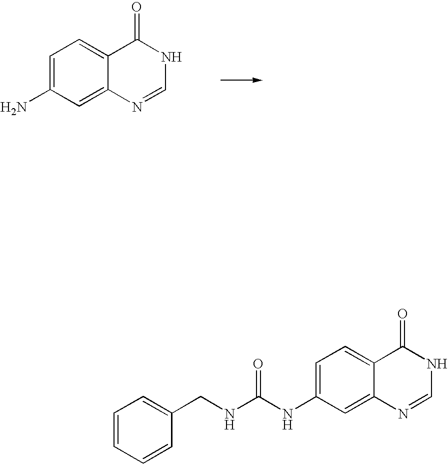

7-Amino-3H-quinazolin-4-one (0.2 g, 1.24 mmol) was reacted with benzyl isocyanate (0.153 ml, 1.24 mmol) in 1,4-dioxane (2 ml) following the procedure set out in Example 13A. Following ion exchange chromatography, the product was further purified by flash silica chromatography, eluting with 2N ammonia in methanol/dichloromethane (10:90). The product was triturated in methanol then filtered to yield the title compound as a white solid (0.069 g, 20% yield). LC/MS: (PS-A2) Rₜ 2.18 [M+H]⁺ 295.02. ¹H NMR (d₆-DMSO) [symbol] 4.34 (2H, d), 6.84 (1H, br m), 7.23-7.28 (1H, m), 7.31-7.38 (4H, m), 7.44 (1H, d), 7.83 (1H, s), 7.97 (1H, d), 8.00 (1H, s), 9.11 (1H, br s), 11.96 (1H, br s).

### Example 9

### 7-(3-Amino-propoxy)-3H-quinazolin-4-one

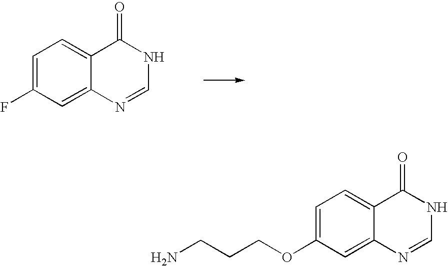

7-Fluoro-3H-quinazolin-4-one (0.050 g, 0.30 mmol) was reacted with 3-amino-1-propanol (0.093 ml, 1.22 mmol) following the procedure set out in Example 12. Following work-up the product was dissolved in water (2 ml) and 4N HCl in 1,4-dioxane was added (2 ml). The solution was heated at 100° C. for 2 hours and then the solvent was removed under reduced pressure. The residue was purified by ion exchange chromatography followed by flash silica chromatography, eluting with a gradient of methanol/dichloromethane (10:90 to 30:70) to yield the title compound as a colourless gum (0.0114 g, 17% yield). LC/MS: (PS-P) Rₜ 1.52 [M+H]⁺ 220.03. ¹H NMR (Me-d₃-OD) [symbol] 2.00-2.08 (2H, m), 2.92 (2H, t), 4.23 (2H, t), 7.09-7.16 (2H, m), 8.09 (1H, s), 8.13 (1H, d).

### Example 10

### 7-(2-Amino-ethoxy)-3H-quinazolin-4-one

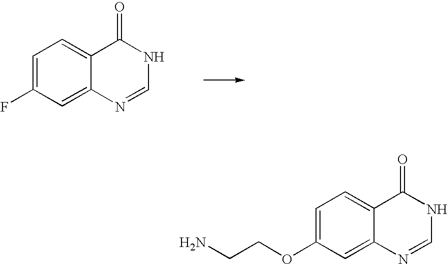

7-Fluoro-3H-quinazolin-4-one (0.075 g, 0.457 mmol) was reacted with ethanolamine (0.110 ml, 1.83 mmol) following the procedure set out in Example 9. Following evaporation of water and 1,4-dioxane, the residue was purified by ion exchange chromatography followed by flash silica chromatography eluting with 2N ammonia in methanol/dichloromethane (20:80) to yield the title compound as a white solid (0.0039 g, 4% yield). LC/MS: (PS-P) Rₜ 1.05 [M+H]⁺ 205.97. ¹H NMR (Me-d₃-OD) [symbol] 3.16 (2H, t), 4.22 (2H, t), 7.17 (1H, s), 7.22 (1H, d), 8.09 (1H, s), 8.16 (1H, d).

### Example 11

### 4-Oxo-3,4-dihydro-quinazoline-7-carboxylic acid[3-amino-3-(4-chloro-phenyl)-propyl]-amide

### 11A. {1-(4-Chloro-phenyl)-3-[4-oxo-3,4-dihydro-quinazoline-7-carbonyl)-amino]-propyl}-carbamic acid tert-butyl ester

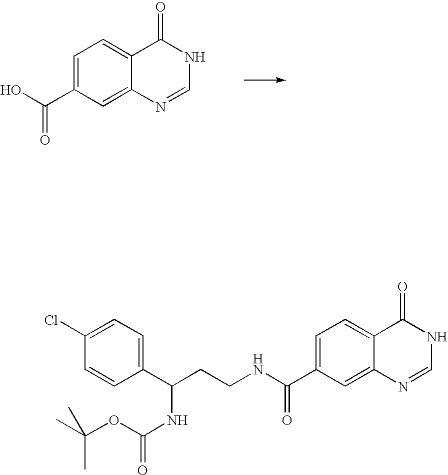

4-Oxo-3,4-dihydro-quinazoline-7-carboxylic acid (0.2 g, 1.05 mmol) was reacted with [3-amino-1-(4-chloro-phenyl)-propyl]-carbamic acid tert-butyl ester (0.3 g, 1.05 mmol) (Pharmacore, 550213) following the procedure set out in Example 3D except that a smaller proportion of N-ethyl-diisopropylamine used (0.136 g, 1.05 mmol). After work-up the product was purified by flash silica chromatography, eluting with methanol/dichloromethane (10:90) to yield the title compound as a white solid (0.299 g, 62% yield). LC/MS: (PS-B2) Rₜ 2.74 [M+H]⁺ 457.03.

### 11B. 4-Oxo-3,4-dihydro-quinazoline-7-carboxylic acid[3-amino-3-(4-chloro-phenyl)-propyl]-amide

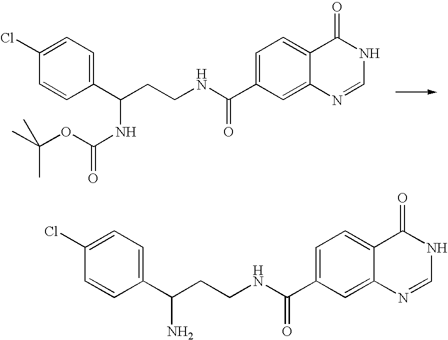

{1-(4-Chloro-phenyl)-3-[(4-oxo-3,4-dihydro-quinazoline-7-carbonyl)-amino]-propyl}-carbamic acid tert-butyl ester (0.3 g, 0.66 mmol) was converted to 4-oxo-3,4-dihydro-quinazoline-7-carboxylic acid [3-amino-3-(4-chloro-phenyl)-propyl]-amide using the same procedure as described for Example 1B except that the reaction time was 16 hours. The title compound was obtained as a glassy colourless solid (0.178 g, 76% yield). LC/MS: (PS-B2) Rₜ 2.16 [M+H]⁺ 356.97. ¹H NMR (Me-d₃-OD) [symbol] 2.07 (2H, q), 3.41-3.49 (2H, m), 4.01 (1H, t), 7.33 (2H, d), 7.39 (2H, d), 7.86 (1H, d), 8.06 (1H, s), 8.15 (1H, s), 8.27 (1H, d).

### Example 12

### 7-(2-Dimethylamino-ethoxy)-3H-quinazolin-4-one

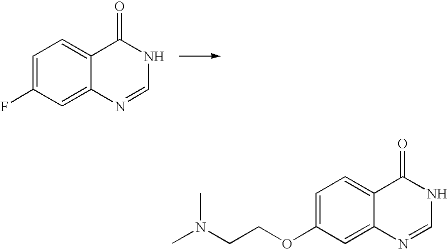

N,N-dimethylethanolamine (0.184 ml, 1.83 mmol) was dissolved in anhydrous N,N-dimethylformamide (1.14 ml) with stirring. The solution was cooled to 0° C. with stirring for 10 minutes and then sodium hydride (60% dispersion in oil, 0.08 g, 2.01 mmol) was added. The resulting suspension was warmed to room temperature and stirred for 1 hour. To this was added a solution of 7-fluoro-3H-quinazolin-4-one (0.75 g, 0.457 mmol) in anhydrous N,N-dimethylformamide (1.1 ml). The reaction mixture was stirred at 140° C. for 2 hours. The reaction mixture was cooled to room temperature, diluted with saturated sodium bicarbonate solution and extracted three times with ethyl acetate. The organic layer was dried (MgSO₄) and solvent was removed under reduced pressure to yield the title compound as a yellow solid (0.09 g, 84% yield). LC/MS: (PS-A2) Rₜ 0.60 [M+H]⁺ 234.01. ¹H NMR (d₆-DMSO) [symbol] 2.23 (6H, s), 2.67 (2H, t), 4.20 (2H, t), 7.07-7.13 (2H, m), 7.98-8.08 (2H, m), 12.08 (1H, br s).

### Example 13

### 1-(4-Chloro-phenyl)-3-(4-oxo-3,4-dihydro-quinazolin-7-yl)-urea

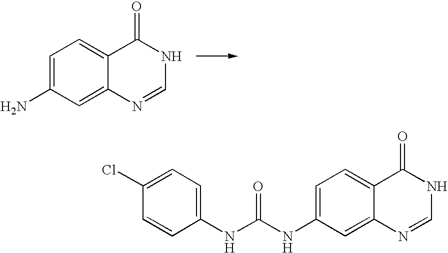

7-Amino-3H-quinazolin-4-one (0.2 g, 1.24 mmol) and 4-chlorophenyl isocyanate (0.19 g, 1.24 mmol) were mixed together in a reaction vial and suspended in 1,4-dioxane (2 ml). The reaction was sealed and heated at 100° C. for 1.5 hours. The suspension was filtered and the solid was washed with methanol followed by diethyl ether. The solid was purified by ion exchange chromatography to yield the title compound as a white solid (0.054 g, 14% yield). LC/MS: (PS-B1) Rₜ 2.47 [M+H]⁺ 314.96. ¹H NMR (d₆-DMSO) [symbol] 7.33-7.37 (2H, m), 7.49-7.55 (4H, m), 7.85 (1H, s), 7.97 (1H, br s), 8.02 (1H, d), 8.97 (1H, br s), 9.21 (1H, br s).

### Example 14

### 7-(3-Amino-propyl)-3H-quinazolin-4-one

### 14A. 2-Amino-4-bromo-benzoic acid

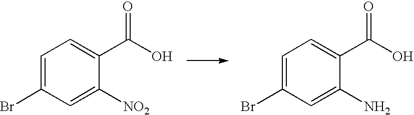

4-Bromo-2-nitro-benzoic acid (0.5 g, 2.03 mmol) (Matrix, 009241) was dissolved in a 1:1 mixture of ethanol/tetrahydrofuran (22 ml). This solution was added to 5% platinum on carbon (0.2 g, 50% water content) under an atmosphere of nitrogen. The reaction was shaken under an atmosphere of hydrogen for 2.5 hours. A further batch of platinum on carbon was added (0.2 g) and the mixture was shaken for 64 hours under an atmosphere of hydrogen. The reaction mixture was filtered, washing through with a 1:1 mixture of ethanol/tetrahydrofuran. The solvent was removed under reduced pressure and the residue was purified by flash silica chromatography, eluting with methanol/dichloromethane (2:98) to yield the title compound as a yellow solid (0.253 g, 58%). LC/MS: (PS-A1) Rₜ 2.62 [M+H]⁺ 215.88.

### 14B. 7-Bromo-3H-quinazolin-4-one

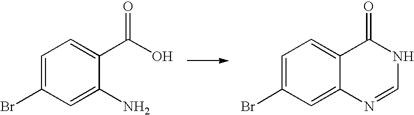

2-Amino-4-bromo-benzoic acid (0.5 g, 2.31 mmol) was converted to 7-bromo-3H-quinazolin-4-one using the same procedure as described for Example 7A to yield the title compound as a beige solid (0.285 g, 55% yield). LC/MS: (PS-A2) Rₜ 2.20 [M+H]⁺ 224.88

### 14C. Prop-2-ynyl-carbamic acid tert-butyl ester

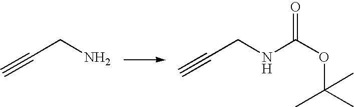

Di-tert-butyl dicarbonate (19.8 g, 90.8 mmol) was dissolved in anhydrous dichloromethane (36 ml) and then added dropwise over 15 minutes to a solution of prop-2-ynylamine (6.22 ml, 90.8 mmol) in anhydrous dichloromethane (36 ml) at 0° C. The resulting solution was stirred at room temperature for 3 hours. The solvent was removed under reduced pressure to leave a liquid that crystallized on standing. The solid was triturated with petroleum ether, filtered then dried to yield the title compounds as a yellow crystalline solid (2.45 g, 17% yield). ¹H NMR (CDCl₃) [symbol] 1.47 (9H, s), 2.23 (1H, t), 3.94 (2H, br s).

### 14D. [3-(4-Oxo-3,4-dihydro-quinazolin-7-yl)-prop-2-ynyl]-carbamic acid tert butyl ester

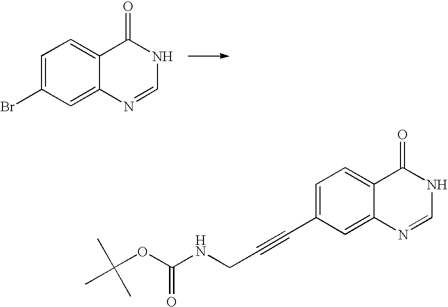

7-Bromo-3H-quinazolin-4-one (0.38 g, 1.69 mmol) was mixed with copper iodide (0.0456 g, 0.239 mmol). The mixture was suspended in anhydrous N,N-dimethylformamide (9.12 ml) and triethylamine was added (6.08 ml, 43.3 mmol). The solution was degassed and bis(triphenylphosphine)palladium(II)chloride (0.0228 g, 0.032 mmol) was added followed by prop-2-ynyl-carbamic acid tert-butyl ester (0.258 g, 1.66 mmol). The solution was heated at 55° C. with stirring for 18 hours under nitrogen. The solvent was removed under reduced pressure and the residue was dissolved in dichloromethane and washed with water. A solid precipitated out and was filtered then washed with dichloromethane and water. The solid was dried under vacuum to yield the title compound that was used in the next step without purification (0.178 g, 35% yield). LC/MS: (PS-A2) Rₜ 2.50 [M+H]⁺ 300.05.

### 14E. [3-(4-Oxo-3,4-dihydro-quinazolin-7-yl)-propyl]-carbamic acid tert-butyl ester

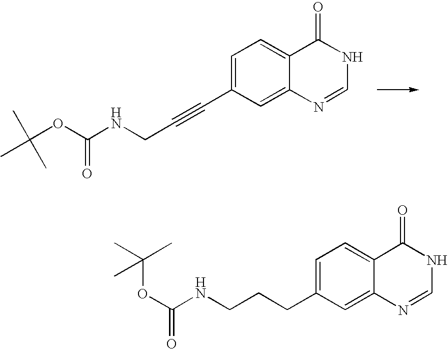

[3-(4-Oxo-3,4-dihydro-quinazolin-7-yl)-prop-2-ynyl]-carbamic acid tert butyl ester (0.035 g, 0.117 mmol) was suspended in ethanol (2.5 ml) and a slurry of Raney nickel in water was added (approximately 0.5 ml). The reaction was shaken under an atmosphere of hydrogen for 18 hours. The reaction mixture was filtered through Celite and solvent was removed under reduced pressure to yield the title compound as a white solid (0.0212 g, 60% yield). LC/MS: (PS-B1) Rₜ 2.40 [M+H]⁺ 304.08.

### 14F. 7-(3-Amino-propyl)-3H-quinazolin-4-one

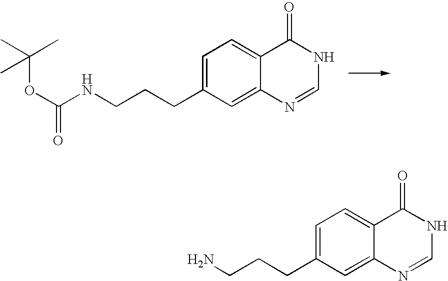

[3-(4-Oxo-3,4-dihydro-quinazolin-7-yl)-propyl]-carbamic acid tert-butyl ester (0.0212 g, 0.0698 mmol) was converted to 7-(3-amino-propyl)-3H-quinazolin-4-one using the same procedure as described in Example 1B except that the product was purified by ion exchange chromatography followed by flash silica chromatography, eluting with a gradient of 2N ammonia in methanol/dichloromethane (20:80 to 30:70). The title compound was afforded as a colourless gum (0.0066 g, 46% yield). LC/MS: (PS-B1) Rₜ 1.58 [M+H]⁺ 203.99. ¹H NMR (Me-d₃-OD) [symbol] 1.75-1.83 (2H, m), 2.64 (2H, t), 2.74 (2H, t), 7.32 (1H, d), 7.41-7.43 (1H, br m), 8.00 (1H, s), 8.04 (1H, d).

### Example 15

### 7-(trans-4-Amino-cyclohexylamino)-3H-quinazolin-4-one

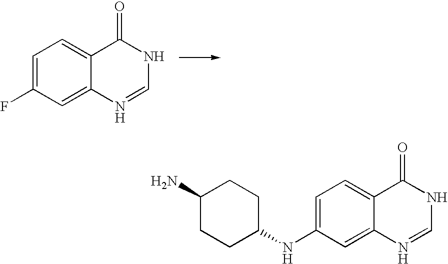

7-Fluoro-3H-quinazolin-4-one (0.075 g, 0.457 mmol) was weighed into a microwave tube followed by 1,4-trans diaminocyclohexane (0.209 g, 1.83 mmol). The mixture was suspended in water (1.5 ml). The suspension was heated in a CEM Explorer™ microwave at 175° C. with stirring for 15 minutes using 100 Watts of power. The reaction mixture was then cooled to room temperature and solvent was removed under reduced pressure. The residue was purified by ion exchange chromatography followed by flash silica chromatography, eluting with 2N ammonia in methanol/dichloromethane (20:80) to yield the title compound as a white solid (0.045 g, 38% yield). LC/MS: (PS-P) Rₜ 1.55 [M+H]⁺ 259.01. ¹H NMR (Me-d₃-OD) [symbol] 1.25-1.40 (4H, m), 1.90-2.02 (2H, m), 2.08-2.17 (2H, m), 2.67-2.75 (1H, m), 3.33-3.41 (1H, m), 6.65 (1H, s), 6.82 (1H, d), 7.90 (1H, d), 7.95 (1H, s).

### Example 16

### 7-(Pyrrolidin-3-ylamino)-3H-quinazolin-4-one

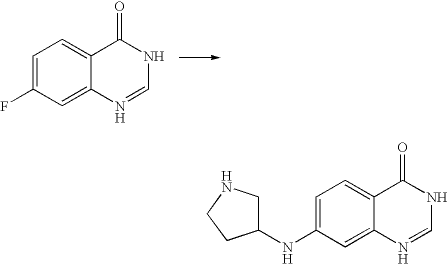

7-Fluoro-3H-quinazolin-4-one (0.075 g, 0.457 mmol) was reacted with 3-amino-pyrrolidine-1-carboxylic acid tert-butyl ester (0.34 g, 1.83 mmol) using the same procedure as described in Example 15 except that the product was purified by ion exchange chromatography followed by flash silica chromatography, eluting with a gradient of 2N ammonia in methanol/dichloromethane (10:90 to 20:80). The title compound was afforded as a yellow solid (0.056 g, 53% yield). LC/MS: (PS-B2) Rₜ 1.73 [M+H]⁺ 231.10. ¹H NMR (d₆-DMSO) [symbol] 1.76-1.84 (1H, m), 2.07-2.16 (1H, m), 3.03-3.08 (1H, m), 3.20-3.42 (1H, m), 3.46-3.54 (2H, m), 3.63-3.69 (1H, m), 6.50 (1H, s), 6.74 (1H, d), 7.87 (1H, d), 7.91 (1H, s).

### Example 17

### 7-(4-Amino-piperidin-1-yl)-3H-quinazolin-4-one

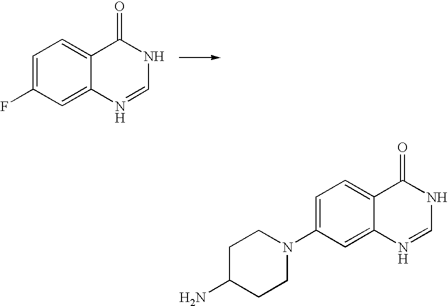

7-Fluoro-3H-quinazolin-4-one (0.075 g, 0.457 mmol) was reacted with piperidin-4-yl-carbamic acid tert-butyl ester (0.366 g, 1.83 mmol) using the same procedure as described in Example 15 except that the product was purified by ion exchange chromatography followed by flash silica chromatography, eluting with a gradient of 2N ammonia in methanol/dichloromethane (20:80 to 30:70). The product was converted to the dihydrochloride salt by following the procedure described in Example 3E. The title compound was afforded as a yellow solid (0.05 g, 34% yield). LC/MS: (PS-P) Rₜ 1.57 [M+H]⁺ 245.07. ¹H NMR (Me-d₃-OD) [symbol] 1.67-1.78 (2H, m), 2.16-2.21 (2H, m), 3.14-3.22 (2H, m), 3.44-3.53 (1H, m), 4.19-4.26 (2H, m), 7.01 (1H, s), 7.40 (1H, d), 8.11 (1H, d), 9.08 (1H, s).

### Example 18

### 7-piperazin-1-yl-3H-quinazolin-4-one

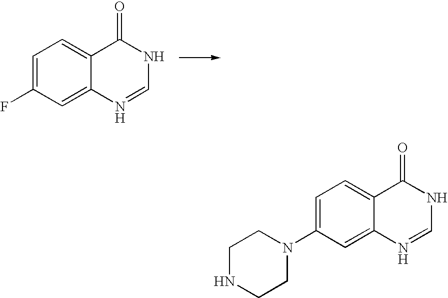

7-Fluoro-3H-quinazolin-4-one (0.05 g, 0.3 mmol) was reacted with piperazine (0.105 g, 1.22 mmol) using the same procedure as described in Example 15 except that the product precipitated from solution upon cooling to room temperature. The product was filtered, washed with water followed by diethyl ether and then dried. The product was converted to the dihydrochloride salt by following the procedure described in Example 3E. The title compound was afforded as a beige solid (0.044 g, 48% yield). LC/MS: (PS-A2) Rₜ 0.48 [M+H]⁺ 231.08H NMR (d₆-DMSO) [symbol] 3.22 (4H, br s), 3.66 (4H, br s), 6.99-7.42 (2H, br m), 7.98 (1H, br s), 8.76 (1H, br s), 9.60 (2H, br s).

### Example 19

### 7-[1,4]Diazepan-1-yl-3H-quinazolin-4-one

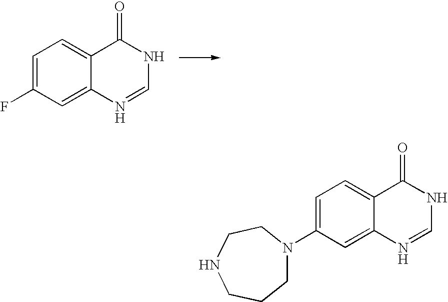

7-Fluoro-3H-quinazolin-4-one (0.050 g, 0.3 mmol) was reacted with [1,4]diazepane (0.120 g, 1.2 mmol) using the same procedure as described in Example 15. Following the reaction, the precipitate that had formed was filtered and dried yielding the title compound (0.026 g, 36% yield). LCMS: (PS-B2), Rₜ 1.68 [M+H]⁺, 245.00. ¹H NMR (Me-d₃-OD) [symbol] 1.97-2.03 (2H, m), 2.84 (2H, t), 3.06 (2H, t), 3.71-3.76 (4H, m), 6.833 (1H, d), 7.067 (1H, d), 7.09 (1H, d), 7.97 (1H, s), 8.027 (1H, d).

### Example 20

### 7-(Piperidin-3-ylamino)-3H-quinazolin-4-one

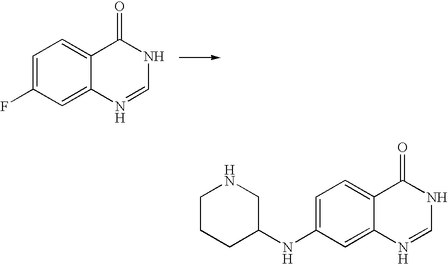

7-Fluoro-3H-quinazolin-4-one (0.075 g, 0.457 mmol) was reacted with 3-amino-piperidine-1-carboxylic acid tert-butyl ester (0.366 g, 1.83 mmol) using the same procedure as described in Example 15 except that the product was purified by ion exchange chromatography followed by flash silica chromatography, eluting with 2N ammonia in methanol/dichloromethane (20:80). The title compound was afforded as a yellow glassy solid (0.030 g, 27% yield). LC/MS: (PS-B2) Rₜ 3.38 [M+H]⁺ 245.131H NMR (Me-d₃-OD) [symbol] 1.37-1.47 (1H, m), 1.62-1.74 (1H, m), 1.82-1.91 (1H, m), 2.00-2.08 (1H, m), 2.79-3.05 (3H, m), 3.78-3.86 (1H, m), 3.91-3.97 (1H, m), 6.96-6.99 (1H, m), 7.19-7.24 (1H, m), 7.99-8.04 (2H, m).

### Example 21

### 7-(4-Amino-cyclohexylamino)-1H-quinazoline-2,4-dione

### 21A. 7-Fluoro-1H-quinazoline-2,4-dione

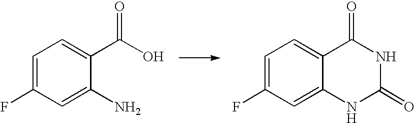

2-Amino-4-fluoro benzoic acid (1 g, 6.45 mmol) and urea (5.96 g, 99 mmol) were mixed together as solids and heated at 160° C. with stirring for 2 hours. The reaction mixture was then heated at 180° C. for a further 1.5 hours. The reaction mixture was allowed to cool to room temperature and stand for 18 hours. The hard solid residue was suspended in methanol and allowed to stand for 64 hours. The residue was triturated and filtered, washing with methanol. The product was suspended in 2N aqueous sodium hydroxide (100 ml) and heated with a hot air gun to give a fine suspension. The suspension was acidified to pH 1 with concentrated HCl causing a precipitate to form. The solid was filtered, washed with water and methanol and dried to yield the title compound as a beige solid (0.645 g, 56% yield). LC/MS: (PS-P) Rₜ 2.18 [M−H]⁻ 178.97.

### 21B. 7-(trans-4-Amino-cyclohexylamino)-1H-quinazoline-2,4-dione

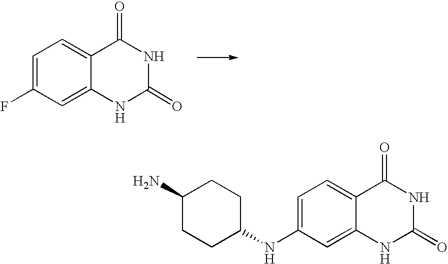

7-Fluoro-1H-quinazoline-2,4-dione (0.075 g, 0.416 mmol) was reacted with 1,4-trans diaminocyclohexane (0.19 g, 1.66 mmol) using the same procedure as described in Example 15 except that the reaction mixture was heated for a further 2 hours at 175° C. in a sealed reaction vial after the microwave reaction. After cooling to room temperature, the reaction mixture was diluted with water to give a suspension that was filtered, washing with water and diethyl ether. The aqueous was isolated and the water was removed under reduced pressure. The residue was purified by ion exchange chromatography, followed by flash silica chromatography eluting with a gradient of 2N ammonia in methanol/dichloromethane (20:80 to 30:70) to afford the title compound as a white solid (0.0179 g, 16%). LC/MS: (PS-P) Rₜ 1.65 [M+H]⁺ 275.09 ¹H NMR (d₆-DMSO) [symbol] 1.05-1.26 (4H, m), 1.75-1.83 (2H, m), 1.88-1.96 (2H, m), 2.50-2.59 (1H, m), 3.06-3.17 (1H, m), 6.14 (1H, s), 6.39 (1H, d), 6.57 (1H, d), 7.52 (1H, d).

### Example 22

### 7-(4-Methyl-[1,4]diazepan-1-yl)-3H-quinazolin-4-one

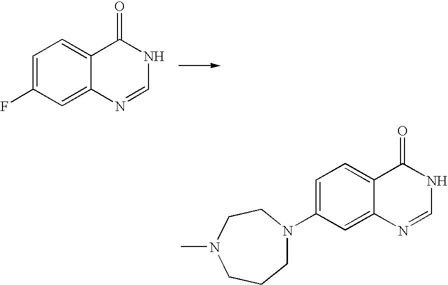

7-Fluoro-3H-quinazolin-4-one (0.050 g, 0.3 mmol) was reacted with 1-methyl-[1,4]diazepane (0.151 ml, 1.2 mmol) using the same procedure as described in Example 15. Following the reaction, the precipitate that had formed was filtered and dried yielding the title compound (0.033 g, 43% yield). LCMS: (PS-B2), Rₜ 1.82 [M+H]⁺, 259.03. ¹H NMR (Me-d₃-OD) [symbol] 1.943-2.002 (2H, m), 2.291 (3H, s), 2.521 (2H, t), 2.70 (2H, t), 3.54 (2H, t), 3.62 (2H, t), 6.696 (1H, d), 6.933 (1H, d), 6.955 (1H, d), 7.86 (1H, s), 7.905 (1 h, d).

### Example 23

### 7-[4-(4-Methyl-piperazin-1-yl)-piperidin-1-yl]-3H-quinazolin-4-one

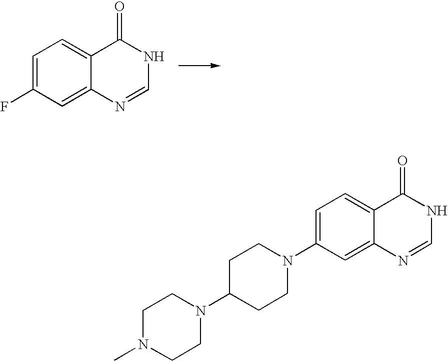

7-Fluoro-3H-quinazolin-4-one (0.050 g, 0.3 mmol) was reacted with 1-methyl-4-piperidin-4-yl-piperazine (0.223 mg, 1.2 mmol) using the same procedure as described in Example 15. Following the reaction, the precipitate that had formed was filtered and dried yielding the title compound (0.060 g, 61% yield). LCMS: (PS-B2), Rₜ 1.83 [M+H]⁺, 328.10. ¹H NMR (Me-d₃-OD) 1.65-1.55 (2H, m), 2.06 (2H, br m), 2.30 (3H, s), 2.50-2.74 (9H, m), 2.97 (2H, t), 4.09-4.13 (2H, m), 6.99 (1H, s), 7.24 (1H, d), 7.99 (1H, s), 8.03 (1H, d)

### Example 24

### 7-(4-Morpholin-4-yl-piperidin-1-yl)-3H-quinazolin-4-one

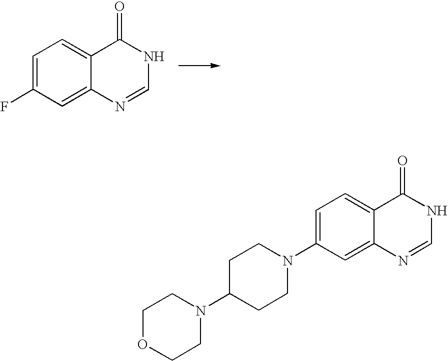

7-Fluoro-3H-quinazolin-4-one (0.050 g, 0.3 mmol) was reacted with 4-piperidin-4-yl-morpholine (0.207 mg, 1.2 mmol) using the same procedure as described in Example 15. Following the reaction, the precipitate that had formed was filtered and dried yielding the title compound (0.074 g, 79% yield). LCMS: (PS-B2), Rₜ 1.96 [M+H]⁺, 315.09. ¹H NMR (Me-d₃-OD) [symbol] 1.40-1.50 (2H, m), 1.88 (2H, br d), 2.46-2.53 (5H, br m), 2.89 (2H, t), 3.57 (4H, t), 3.97 (2H, d), 6.92 (1H, br s), 7.17 (1H, d), 7.88 (1H, d), 7.94 (1H, s)

### Example 25

### 7-(3-Phenyl-piperazin-1-yl)-1H-quinazoline-2,4-dione

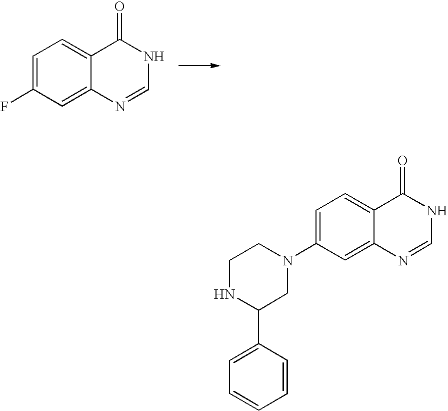

7-Fluoro-3H-quinazolin-4-one (0.075 g, 0.457 mmol) was reacted with 2-phenyl piperazine (0.297 g, 1.83 mmol) using the same procedure as described in Example 18. The title compound was afforded as a brown solid (0.095 g, 55%). LC/MS: (PS-P) Rₜ 1.91 [M+H]⁺ 307.02 ¹H NMR (d₆-DMSO) [symbol] 3.23-3.36 (1H, m), 3.44-3.69 (3H, m), 4.09-4.20 (2H, m), 4.49-4.58 (1H, m), 7.24 (1H, s), 7.40-7.54 (4H, m), 7.74-7.83 (2H, m), 7.98 (1H, d), 8.82-8.90 (1H, m), 9.98 (1H, br s), 10.51 (1H, br s).

### Example 26

### 7-(4-Methyl-piperazin-1-yl)-3H-quinazolin-4-one

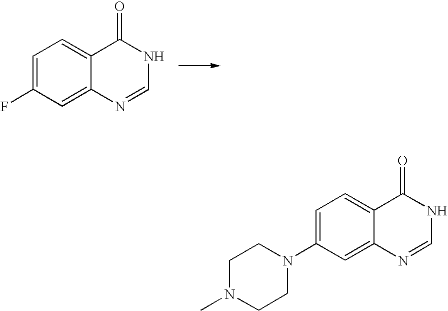

7-Fluoro-3H-quinazolin-4-one (0.05 g, 0.30 mmol) was reacted with 1-methyl piperazine (0.135 ml, 1.22 mmol) using the same procedure as described in Example 18. The title compound was afforded as a beige solid (0.062 g, 65%). LC/MS: (PS-B2) Rₜ 1.85 [M+H]⁺ 245.04 ¹H NMR (d₆-DMSO) [symbol] 2.81 (3H, s), 3.08-3.25 (2H, m), 3.32-3.45 (2H, m), 3.46-3.58 (2H, m), 4.04-4.16 (2H, m), 7.17 (1H, s), 7.34 (1H, d), 7.99 (1H, d), 8.74 (1H, s), 11.48 (1H, br s).

### Example 27

### 7-[4-Aminomethyl-4-(4-chloro-phenyl)-piperidin-1-yl]-3H-quinazolin-4-one

### 27A. Bis-(2-chloro-ethyl)-carbamic acid tert-butyl ester

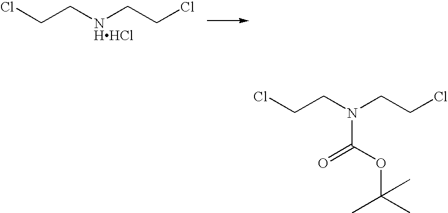

Bis-(2-chloro-ethyl)-carbamic acid tert-butyl ester was made using a method described in J. Chem. Soc., Perkin Trans 1, 2000, p3444-3450.

### 27B. 4-(4-Chloro-phenyl)-4-cyano-piperidine-1-carboxylic acid tert-butyl ester

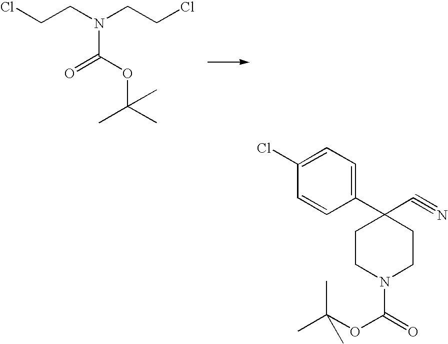

4-(4-Chloro-phenyl)-4-cyano-piperidine-1-carboxylic acid tert-butyl ester was made using a method described in International patent application WO2004022539.

### 27C. 4-Aminomethyl-4-(4-chloro-phenyl)-piperidine-1-carboxylic acid tert-butyl ester

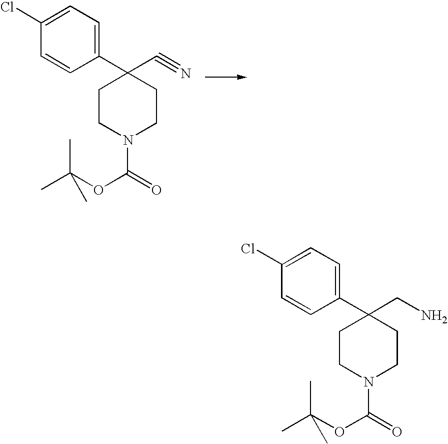

4-(4-Chloro-phenyl)-4-cyano-piperidine-1-carboxylic acid tert-butyl ester (0.4 g, 0.125 mmol) was dissolved in ethanol (52 ml) under an atmosphere of nitrogen. To this solution was added concentrated aqueous ammonia (10.4 ml) followed by a slurry of Raney nickel in water (5.4 ml). The vessel was charged with hydrogen and shaken for 8 hours. The reaction mixture was then filtered through Celite under reduced pressure, washing through with methanol. The filtrate was concentrated under reduced pressure. The residue was purified by ion exchange chromatography followed by flash silica chromatography eluting with 5:95 methanol:dichloromethane to yield the title compound as a colourless gum (0.141 g, 35% yield). LC/MS: (PS-P) Rₜ 2.73 [M+H]⁺ 325.201H NMR (Me-d₃-OD) [symbol] 1.46 (9H, s), 1.68-1.76 (2H, m), 2.15-2.25 (2H, m), 2.73 (2H, s), 2.93-3.10 (2H, m), 3.72-3.81 (2H, m), 7.37-7.45 (4H, m).

### 27D. 7-[4-Aminomethyl-4-(4-chloro-phenyl)-piperidin-1-yl]-3H-quinazolin-4-one

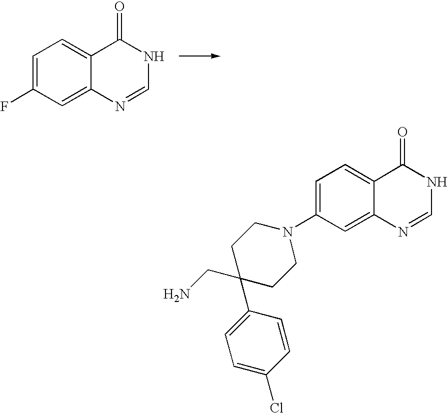

7-Fluoro-3H-quinazolin-4-one (0.024 g, 0.144 mmol) was reacted with 4-aminomethyl-4-(4-chloro-phenyl)-piperidine-1-carboxylic acid tert-butyl ester (0.14 g, 0.43 mmol) using the same procedure as described in Example 15 except that once the reaction was complete and had cooled to room temperature the reaction mixture was diluted with water and extracted three times with ethyl acetate. The organics were dried (MgSO₄), filtered and solvent was removed under reduced pressure. The residue was purified by flash silica chromatography, eluting with methanol:dichloromethane (20:80) to yield the title compound as a white solid (0.028 g, 53% yield). LC/MS: (PS-P) Rₜ 2.48 [M+H]⁺ 369.33 ¹H NMR (d₆-DMSO) [symbol] 1.84-1.93 (2H, m), 2.13-2.22 (2H, m), 2.65 (2H, s), 3.01-3.09 (2H, m), 3.60-3.68 (2H, m), 6.89 (1H, s), 7.15 (1H, d), 7.39-7.45 (4H, m), 7.86 (1H, d), 7.93 (1H, s).

### Example 28

### (S)-4-Amino-2-(3,4-dichloro-phenyl)-N-(4-oxo-3,4-dihydro-quinazolin-7-yl butyramide

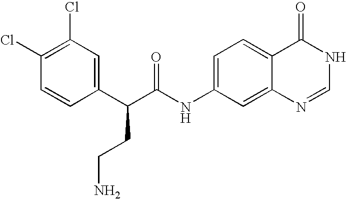

Racemic 4-amino-2-(3,4-dichloro-phenyl)-N-(4-oxo-3,4-dihydro-quinazolin-7-yl)butyramide (0.03 g, 0.077 mmol), prepared according to procedure 1B was purified by preparative chiral HPLC using method (AG-CP) to yield the title compound as a pale yellow solid (0.0028 g, 19% yield assuming 1:1 racemic mixture). LC: (AG-CA) Rₜ 9.660 (95% ee).

### Example 29

### (R)-4-Amino-2-(3,4-dichloro-phenyl)-N-(4-oxo-3,4-dihydro-quinazolin-7-yl)butyramide

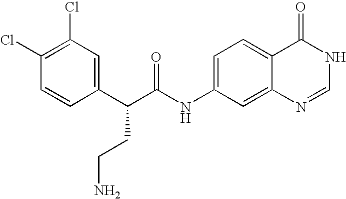

Racemic 4-amino-2-(3,4-dichloro-phenyl)-N-(4-oxo-3,4-dihydro-quinazolin-7-yl)butyramide (0.03 g, 0.077 mmol), prepared according to procedure 1B was purified by preparative chiral HPLC using method (AG-CP) to yield the title compound as a pale yellow solid (0.0033 g, 22% yield assuming 1:1 racemic mixture). LC: (AG-CA) Rₜ 10.609 (95% ee).

### Example 30

### 4-(4-Chloro-phenyl)-piperidine-4-carboxylic acid (4-oxo-3,4-dihydro-quinazolin-7-yl)-amide

### 30A. 4-(4-Chloro-phenyl)-piperidine-4-carbonitrile

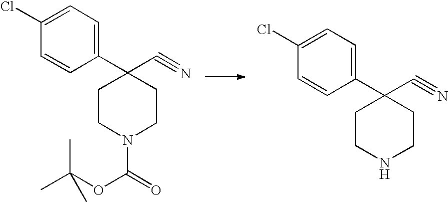

4-(4-Chloro-phenyl)-piperidine-4-carbonitrile was made using a method described in International patent application WO2004/022539.

### 30B. 4-Carboxy-4-(4-chloro-phenyl)-piperidinium chloride

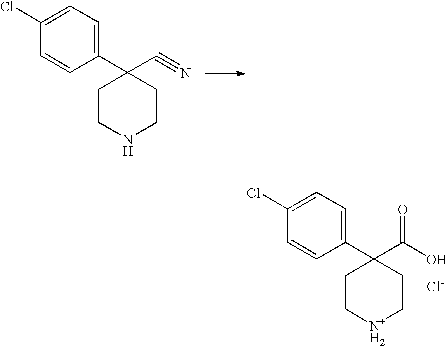

4-(4-Chloro-phenyl)-piperidine-4-carbonitrile (1.11 g, 5.03 mmol) was suspended in 8N aqueous HCl (66 ml). The suspension was heated at 100° C. for a total of 46 hours after which the reaction was allowed to cool to room temperature and stirred for a further 65 hours. The solvent was removed under reduced pressure to afford the title compound as a beige solid (1.60 g, residual water present). The product was carried forward without purification. LC/MS: (PS-P) Rₜ 2.44 [M+H]⁺ 240.17.

### 30C. 4-(4-Chloro-phenyl)-piperidine-1,4-dicarboxylic acid mono-(9H-fluoren-9-ylmethyl)ester

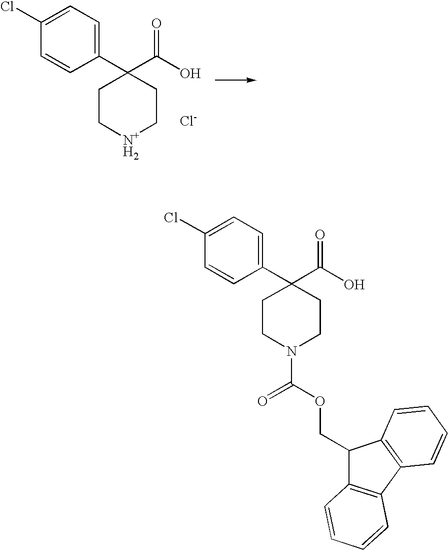

4-Carboxy-4-(4-chloro-phenyl)-piperidinium chloride (0.5 g, 1.81 mmol) was reacted with carbonic acid 2,5-dioxo-pyrrolidin-1-yl ester 9H-fluoren-9-ylmethyl ester (0.672 g, 1.99 mmol) using the same procedure as described in Example 4A except that the reaction time was 5 hours. The reaction mixture was diluted with 1N HCl (aq) and the aqueous mixture was extracted three times with ethyl acetate. The organic extracts were combined, dried (MgSO₄) and solvent was removed under reduced pressure. The residue was purified by flash silica chromatography, eluting with a gradient of methanol/dichloromethane (100% dichloromethane to 1:99 to 10:90 with 1% acetic acid) to afford the title compound as an off-white foam (0.361 g, 43%). LC/MS: (PS-A2) Rₜ 4.02 [M+H]⁺ 462.21.

### 30D. 4-Chlorocarbonyl-4-(4-chloro-phenyl)-piperidine-1-carboxylic acid 9H-fluoren-9-ylmethyl ester

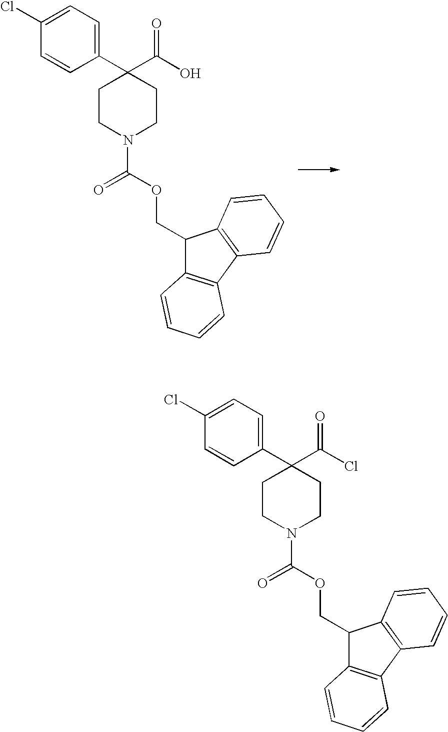

4-(4-Chloro-phenyl)-piperidine-1,4-dicarboxylic acid mono-(9H-fluoren-9-ylmethyl)ester (0.18 g, 0.39 mmol) was reacted with thionyl chloride (4 ml) using the same procedure as described in Example 4B except that the reaction time was 3 hours. Yield: yellow gum (quantitative). LC/MS (in methanol): (PS-A2) Rₜ 4.07 [M+H]⁺ 476.33 (methyl ester).

### 30E. 4-(4-Chloro-phenyl)-4-(4-oxo-3,4-dihydro-quinazolin-7-ylcarbamoyl) piperidine-1-carboxylic acid 9H-fluoren-9-ylmethyl ester

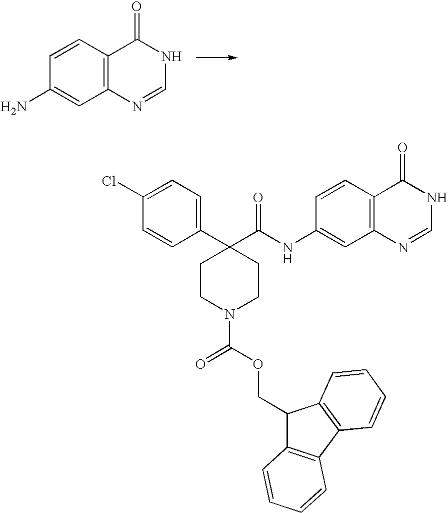

4-Chlorocarbonyl-4-(4-chloro-phenyl)-piperidine-1-carboxylic acid 9H-fluoren-9-ylmethyl ester (0.201 g, 0.42 mmol) was reacted with 7-amino-3H-quinazolin-4-one (0.062 g, 0.38 mmol) using the same procedure as described in Example 4C. The residue after work-up was purified by flash silica chromatography, eluting with a gradient of methanol/ethyl acetate (0.5:99.5 to 1:99) to afford the title compound as a white solid (0.015 g, 6%). LC/MS (PS-B2) Rₜ 3.46 [M+H]⁺ 605.31.

### 30F. 4-(4-Chloro-phenyl)-piperidine-4-carboxylic acid (4-oxo-3,4-dihydro-quinazolin-7-yl)-amide

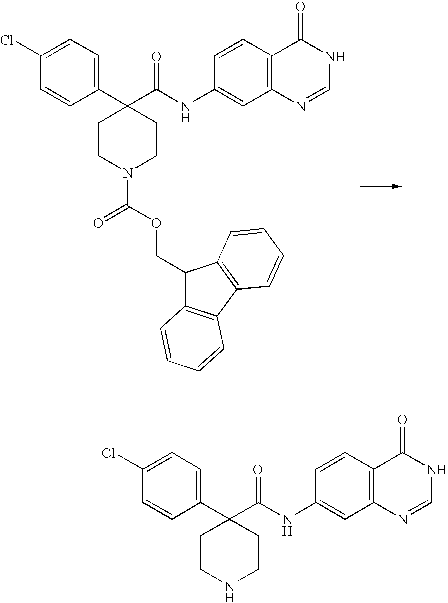

4-(4-Chloro-phenyl)-4-(4-oxo-3,4-dihydro-quinazolin-7-ylcarbamoyl)piperidine-1-carboxylic acid 9H-fluoren-9-ylmethyl ester (0.031 g, 0.051 mmol) was reacted with N-(2-mercaptoethyl)aminomethyl polystyrene (0.128 g, 0.256 mmol) and 1,8-diazabicyclo[5.4.0]undec-7-ene (0.0077 ml, 0.051 mmol) using the same procedure as described in Example 4D except that the reaction mixture was filtered after 18 hours. Solvent was removed under reduced pressure and the residue was again subjected to the reaction conditions above (using the same quantities). The reaction was continued for 69 hours and then filtered, washing through with methanol and dichloromethane. Solvent was removed under reduced pressure and the residue was purified by ion exchange chromatography followed by flash silica chromatography, eluting with a gradient of 2N ammonia in methanol/dichloromethane (20:80 to 25:75). The product was further purified by preparative liquid chromatography to yield the title compound as a glassy, colourless solid (0.004 g, 20%). LC/MS (PS-A2) Rₜ 1.94 [M+H]⁺ 383.32. ¹H NMR (Me-d₃-OD) δ 1.98-2.09 (2H, m), 2.55-2.65 (2H, m), 2.95-3.04 (2H, m), 3.06-3.14 (2H, m), 7.28-7.39 (4H, m), 7.51-7.56 (1H, d), 7.91-8.03 (3H, m).

### Example 31

### 7-(4-Amino-cyclohexyloxy)-3H-quinazolin-4-one

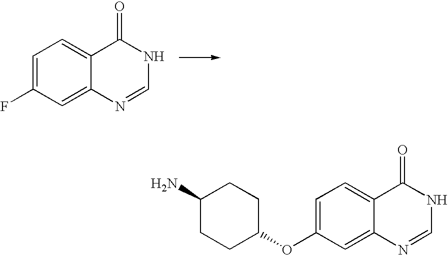

7-Fluoro-3H-quinazolin-4-one (0.050 g, 0.305 mmol) was reacted with trans-4-amino-cyclohexanol (0.150 g, 1.22 mmol) following the procedure set out in Example 7C except that after work-up, heating with potassium hydroxide (0.468 g, 8.34 mmol) was continued for 20 hours. After cooling to room temperature the solvent was removed under reduced pressure and the residue was purified by ion exchange chromatography followed by flash silica chromatography, eluting with a gradient of 2N ammonia in methanol/dichloromethane (10:90 to 20:80 to 25:75). The title compound was afforded as a colourless gum (0.0067 g, 8%). LC/MS (PS-P): Rₜ 1.79 [M+H]⁺ 260.09. ¹H NMR (Me-d₃-OD) δ 1.39-1.51 (2H, m), 1.53-1.65 (2H, m), 2.00-2.08 (2H, m), 2.20-2.29 (2H, m), 2.85-2.94 (1H, m), 7.11-7.15 (2H, m), 8.09 (1H, s), 8.13 (1H, d).

### Example 32

### 7-{[4-(4-Chloro-phenyl)-piperidin-4-ylmethyl]-amino}-3H-quinazolin-4-one

### 32A. 7-Bromo-3-(2,4-dimethoxy-benzyl)-3H-quinazolin-4-one

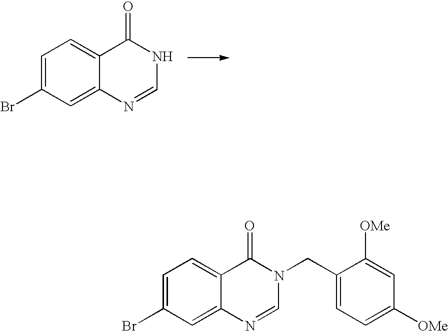

7-Bromo-3H-quinazolin-4-one (0.253 g, 1.12 mmol) was suspended in anhydrous THF (4.5 ml) in a ReactiVial™ (Pierce Chemical Co., Rockford, Ill.). To the suspension was added 2,4-dimethoxybenzyl alcohol (0.377 g, 2.24 mmol) followed by triphenylphosphine (0.588 g, 2.24 mmol). The mixture was cooled to 0° C. with stirring and diethyl azodicarboxylate (0.36 ml, 2.29 mmol) was added dropwise. Stirring was continued at 0° C. for 30 minutes and then the reaction was allowed to warm to room temperature and then heated at 60° C. for 18 hours. The reaction mixture was then allowed to cool to room temperature and diluted with ethyl acetate and brine. The organic layer was separated and washed with aqueous saturated sodium bicarbonate solution. The aqueous was extracted once more and the organics were combined, dried (MgSO₄) and solvent was removed under reduced pressure. The residue was purified by ion exchange chromatography followed by flash silica chromatography, eluting with ethyl acetate/petroleum ether (30:70) to afford the title compound as a white solid (0.111 μg, 26%). LC/MS (PS-A2): Rₜ 3.27 [M+H]⁺ 375.09.

### 32B. 4-(4-Chloro-phenyl)-4-{[3-(2,4-dimethoxy-benzyl)-4-oxo-3,4-dihydro-quinazolin-7-ylamino]-methyl}-piperidine-1-carboxylic acid tert-butyl ester

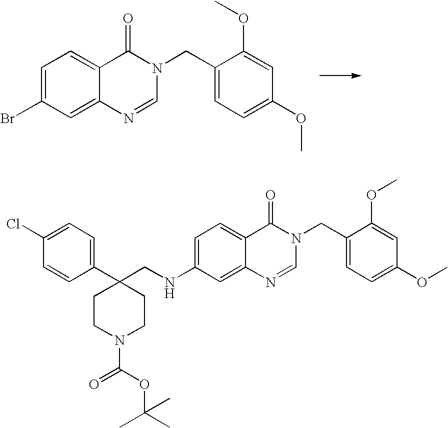

Sodium tert-butoxide (0.0553 g, 0.575 mmol), tris(dibenzylideneacetone) dipalladium (0) (0.0088 g, 0.0096 mmol), and rac-2,2-bis(diphenylphosphino)-1,1-binapthyl (0.012 g, 0.0192 mmol) were combined in an oven-dried Schlenk tube under an atmosphere of nitrogen. 4-Aminomethyl-4-(4-chloro-phenyl)-piperidine-1-carboxylic acid tert-butyl ester (0.187 g, 0.575 mmol) was dissolved in degassed anhydrous 1,4-dioxane (0.8 ml) and this was added to the Schlenk tube. 7-Bromo-3-(2,4-dimethoxy-benzyl)-3H-quinazolin-4-one (0.072 g, 0.192 mmol) was dissolved in degassed anhydrous 1,4-dioxane (0.8 ml) and this was added to the Schlenk tube. The Schlenk tube was evacuated and back-filled with nitrogen and then heated at 65° C. for 3 hours. The reaction mixture was allowed to cool to room temperature and diluted with ethyl acetate and brine. The aqueous was extracted three times with ethyl acetate. The organics were combined, dried (MgSO₄) and solvent was removed under reduced pressure. The residue was purified by flash silica chromatography, eluting with a gradient of ethyl acetate/petroleum ether (60:40 to 70:30) to afford the title compound as a yellow gum (0.113 g, 95%). LC/MS (PS-A2): Rₜ 3.65 [M+H]⁺ 620.44.

### 32C. 7-{[4-(4-Chloro-phenyl)-piperidin-4-ylmethyl]-amino}-3H-quinazolin-4-one

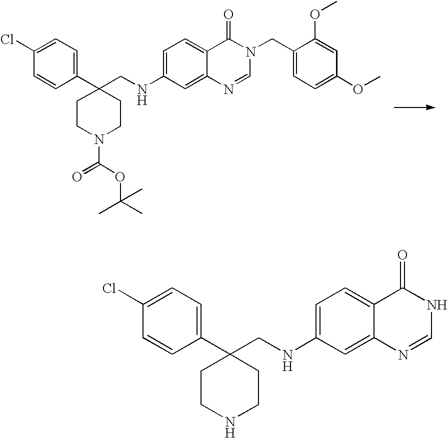

4-(4-Chloro-phenyl)-4-{[3-(2,4-dimethoxy-benzyl)-4-oxo-3,4-dihydro-quinazolin-7-ylamino]-methyl}-piperidine-1-carboxylic acid tert-butyl ester (0.113 g, 0.183 mmol) was dissolved in dichloromethane (2 ml). Water (0.1 ml) was added followed by trifluoroacetic acid (1 ml). The reaction was stirred at room temperature for 1 hour and then solvent was removed under reduced pressure. The residue was dissolved in trifluoroacetic acid (10 ml) and water (0.1 ml) was added. The solution was heated at 50° C. for 5 hours and then solvent was removed under reduced pressure. The residue was purified by ion exchange chromatography followed by flash silica chromatography, eluting with a gradient of methanol/dichloromethane (20:80 to 30:70). The product was further purified by preparative liquid chromatography to yield the title compound as a white solid (0.0248 g, 33%). LC/MS (PS-AE): Rₜ 1.08 [M+H]⁺ 369.27. ¹H NMR (Me-d₃-OD) δ 2.06-2.17 (2H, m), 2.56-2.66 (2H, m), 2.86-2.97 (2H, m), 3.30-3.39 (2H, m), 3.42 (2H, s), 6.48 (1H, s), 6.71 (1H, d), 7.38 (2H, d), 7.48 (2H, d), 7.82 (1H, d), 7.95 (1H, s).

### Example 33

### 7-[4-(4-Chloro-phenyl)-piperidin-4-ylmethoxy]-3H-quinazolin-4-one

### 33A. 4-(4-Chloro-phenyl)-piperidine-4-carboxylic acid ethyl ester

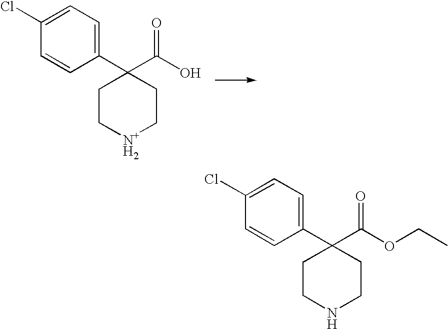

4-Carboxy-4-(4-chloro-phenyl)-piperidinium chloride (1.1 g, 3.98 mmol) was suspended in ethanol (59 ml) and concentrated sulphuric acid was added (0.589 ml). The solution was heated to reflux with stirring for 71 hours. The reaction mixture was then allowed to cool to room temperature and diluted with ethyl acetate and saturated sodium bicarbonate. The aqueous was extracted three times with ethyl acetate. The organics were combined, dried (MgSO₄) and solvent was removed under reduced pressure. The residue was purified by flash silica chromatography, eluting with a gradient of methanol/dichloromethane (2:98 to 5:95 to 10:90) to afford the title compound as a yellow oil (0.789 g, 74%). LC/MS (PS-B2): Rₜ 2.89 [M+H]⁺ 268.25.

### 33B. [4-(4-Chloro-phenyl)-piperidin-4-yl]-methanol

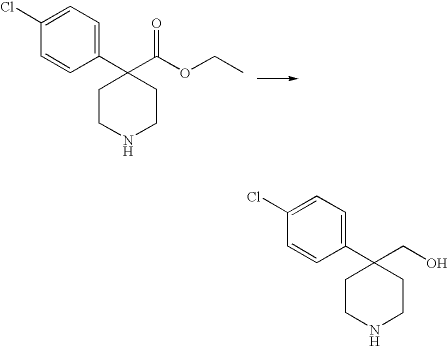

4-(4-Chloro-phenyl)-piperidine-4-carboxylic acid ethyl ester (0.789 g, 2.95 mmol) was suspended in anhydrous diethyl ether (11.5 ml). To this suspension was added dropwise a solution of 1N lithium triethylborohydride in tetrahydrofuran (9.22 ml, 9.22 mmol). The reaction mixture was stirred at room temperature for 1 hour and then a solution of aqueous 1N HCl (23 ml) was added dropwise. After vigorous stirring of the reaction mixture for 3 hours at room temperature, solvent was removed under reduced pressure. The residue was dissolved in saturated sodium bicarbonate and then the water was removed under reduced pressure. The residue was triturated with dichloromethane and then filtered. The filtrate was directly purified by ion exchange chromatography to afford the title compound as a white solid (0.588 g, 88%). LC/MS (PS-B2): Rₜ 2.20 [M+H]⁺ 226.25.

### 33C. 4-(4-Chloro-phenyl)-4-hydroxymethyl-piperidine-1-carboxylic acid tert-butyl ester

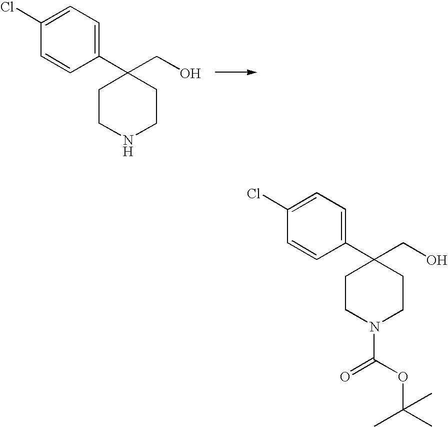

4-(4-Chloro-phenyl)-4-hydroxymethyl-piperidine-1-carboxylic acid tert-butyl ester was made using a method described in International patent application WO2004/022539.

### 33D. 4-(4-Chloro-phenyl)-4-(4-oxo-3,4-dihydro-quinazolin-7-yloxymethyl)-piperidine-1-carboxylic acid tert-butyl ester

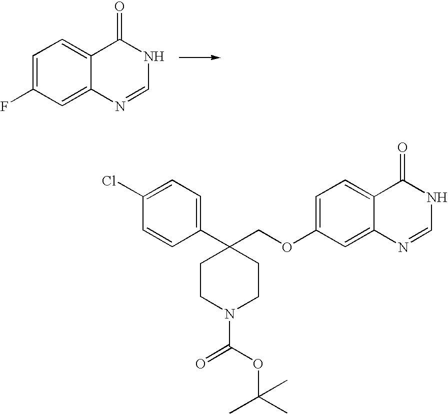

7-Fluoro-3H-quinazolin-4-one (0.0258 g, 0.157 mmol) was reacted with 4-(4-chloro-phenyl)-4-hydroxymethyl-piperidine-1-carboxylic acid tert-butyl ester (0.205 g, 0.629 mmol) following the procedure set out in Example 7C except that the potassium hydroxide step was not carried out. After work-up the residue was purified by flash silica chromatography, eluting with methanol/ethyl acetate (2:98) to afford the title compound as a colourless gum (0.05 g, 68%). LC/MS (PS-B3): Rₜ 3.30 [M+H]⁺ 470.28.

### 33E. 7-[4-(4-Chloro-phenyl)-piperidin-4-ylmethoxy]-3H-quinazolin-4-one

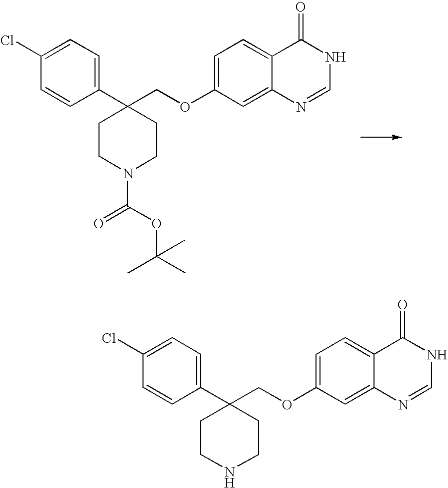

4-(4-Chloro-phenyl)-4-(4-oxo-3,4-dihydro-quinazolin-7-yloxymethyl)piperidine-1-carboxylic acid tert-butyl ester (0.05 g, 0.106 mmol) was suspended in 4N HCl in 1,4-dioxane (10 ml). Methanol (4 ml) was added and the solution was stirred at room temperature for 45 minutes. The solvent was removed under reduced pressure. The residue was purified by purified by ion exchange chromatography followed by flash silica chromatography, eluting with methanol/dichloromethane (20:80) to afford the title compound as a white solid (0.017 g, 44%). LC/MS (PS-BE): Rₜ 5.72 [M+H]⁺ 370.28. ¹H NMR (d₆-DMSO) δ 1.87 (2H, m), 2.07-2.16 (2H, m), 2.54-2.64 (2H, m), 2.79-2.89 (2H, m), 4.10 (2H, s), 6.95-7.00 (1H, m), 7.01-7.05 (1H, m), 7.38-7.44 (2H, m), 7.48-7.54 (2H, m), 7.95 (1H, d), 8.03 (1H, s).

### Example 34

### 7-[4-(4-Chloro-phenyl)-4-hydroxymethyl-piperidin-1-yl]-3H-quinazolin-4-one

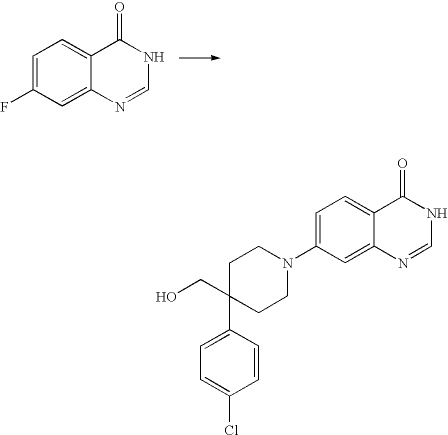

[4-(4-Chloro-phenyl)-piperidin-4-yl]-methanol (0.553 g, 2.45 mmol) was dissolved in anhydrous N,N-dimethylformamide (10 ml). To this solution was added sodium hydride (60% dispersion in oil, 0.145 g, 3.63 mmol) at room temperature. After 15 minutes 7-fluoro-3H-quinazolin-4-one (0.06 g, 0.366 mmol) was added and the mixture was heated at 80° C. with stirring for 2 hours. The reaction mixture was allowed to cool to room temperature and the solvent was removed under reduced pressure. The residue was triturated with methanol and filtered. The filtrate was then concentrated under reduced pressure. The product was purified by preparative liquid chromatography followed by a basic ion exchange column to yield the title compound as a white solid (0.0075 g, 6%). LC/MS (PS-AE): Rₜ 8.74 [M+H]⁺ 370.3. ¹H NMR (Me-d₃-OD) δ 1.98-2.09 (2H, m), 2.27-3.35 (2H, m), 3.06-3.16 (2H, m), 3.55 (2H, s), 3.73-3.82 (2H, m), 6.94 (1H, s), 7.19 (1H, d), 7.35-7.42 (2H, m), 7.43-7.50 (2H, m), 7.97-8.03 (2H, m).

### Example 35

### 7-[4-Aminomethyl-4-(4-chloro-phenyl)-piperidin-1-yl]-2-methyl-3H-quinazolin-4-one

### 35A. 7-Fluoro-2-methyl-3H-quinazolin-4-one

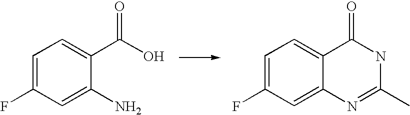

A mixture of 2-amino-4-fluorobenzoic acid (10 g, 64.5 mmol), acetic anhydride (18.26 ml, 193.5 mmol) and heptane (35 ml) was heated to reflux with stirring for 3 hours. Ammonium acetate was added (17.7 g, 229.6 mmol) and the mixture was evaporated under reduced pressure to remove most of the heptane. Acetic acid (53 ml) was added and the mixture was evaporated under reduced pressure until approximately 15 ml of acetic acid remained. The suspension was then heated at reflux for 16 hours. The reaction mixture was allowed to cool to room temperature and then filtered under suction to afford the title compound as a pale yellow crystalline solid, 3.94 g (34%). LC/MS: (PS-B3) Rₜ 1.88 [M+H]⁺ 179.16

### 35B. Bis-(2-chloro-ethyl)-carbamic acid tert-butyl ester

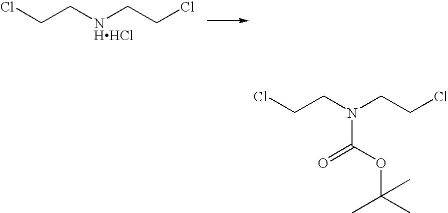

Bis-(2-chloro-ethyl)-carbamic acid tert-butyl ester was made using a method described in J. Chem. Soc., Perkin Trans 1, 2000, p 3444-3450.

### 35C. 4-(4-Chloro-phenyl)-4-cyano-piperidine-1-carboxylic acid tert-butyl ester

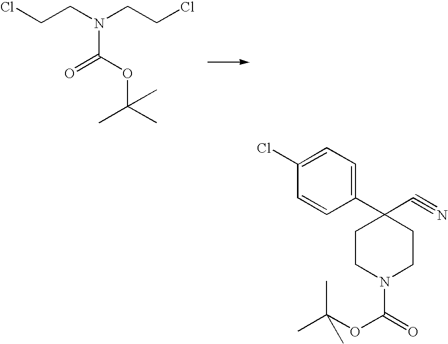

4-(4-Chloro-phenyl)-4-cyano-piperidine-1-carboxylic acid tert-butyl ester was made using a method described in WO2004022539.

### 35D. 4-Aminomethyl-4-(4-chloro-phenyl)-piperidine-1-carboxylic acid tert-butyl ester

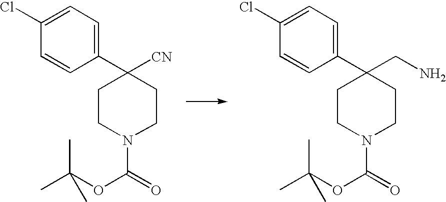

4-(4-Chloro-phenyl)-4-cyano-piperidine-1-carboxylic acid tert-butyl ester (0.4 g, 0.125 mmol) was dissolved in ethanol (52 ml) under an atmosphere of nitrogen. To this solution was added concentrated aqueous ammonia (10.4 ml) followed by a slurry of Raney nickel in water (5.4 ml). The vessel was charged with hydrogen and shaken for 8 hours. The reaction mixture was then filtered through Celite under reduced pressure, washing through with methanol. The filtrate was concentrated under reduced pressure. The residue was purified by ion exchange chromatography followed by flash silica chromatography eluting with 5:95 methanol: dichloromethane to yield the title compound as a colourless gum (0.141 g, 35% yield). LC/MS: (PS-P) Rₜ 2.73 [M+H]⁺ 325.20. ¹H NMR (Me-d₃-OD) [symbol] 1.46 (9H, s), 1.68-1.76 (2H, m), 2.15-2.25 (2H, m), 2.73 (2H, s), 2.93-3.10 (2H, m), 3.72-3.81 (2H, m), 7.37-7.45 (4H, m).

### 35E. 7-[4-Aminomethyl-4-(4-chloro-phenyl)-piperidin-1-yl]-2-methyl-3H-quinazolin-4-one

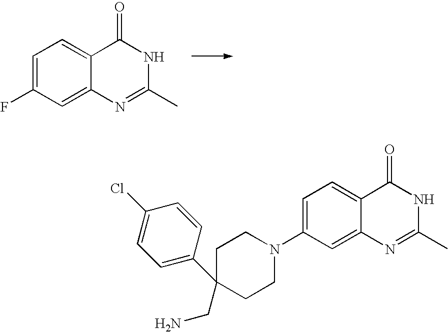

7-Fluoro-2-methyl-3H-quinazolin-4-one (0.050 g, 0.281 mmol) was mixed with 4-aminomethyl-4-(4-chloro-phenyl)-piperidine-1-carboxylic acid tert-butyl ester (0.182 g, 0.561 mmol) in a microwave tube. The mixture was suspended in water (1.0 ml). The suspension was heated in a CEM Explorer™ microwave at 175° C. with stirring for 15 minutes using 100 Watts of power. The reaction mixture was allowed to cool to room temperature and then diluted with water and extracted twice with ethyl acetate. The organics were dried (MgSO₄), filtered and solvent was removed under reduced pressure. The residue was purified by flash silica chromatography, eluting with methanol/dichloromethane (20/80) to afford the title compound as a glassy, colourless solid, 0.028 g (26%). LC/MS: (PS-BE1) Rₜ 5.92 [M+H]⁺ 383.27. ¹H NMR (Me-d₃-OD) δ 1.87-1.98 (2H, m), 2.29-2.40 (2H, m), 2.40 (3H, s), 2.78 (2H, s), 3.05-3.16 (2H, m), 3.66-3.75 (2H, m), 6.84 (1H, br s), 7.11 (1H, br d), 7.38-7.48 (4H, m), 7.95 (1H, d).

### Example 36

### 7-{4-[Amino-(4-chloro-phenyl)-methyl]-piperidin-1-yl}-3H-quinazolin-4-one

### 36A. 7-Fluoro-3H-quinazolin-4-one

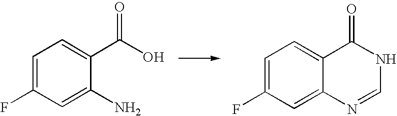

2-Amino-4-fluoro benzoic acid (0.5 g, 3.22 mmol) was suspended in formamide (2 ml) and heated in a CEM Explorer™ microwave at 150° C. with stirring for 15 minutes using 60 Watts of power. Upon cooling to room temperature, a solid precipitated out of solution. The solid was filtered, washing with acetone and then diethyl ether to yield the title compound as a pale grey solid (0.25 g, 47% yield). LC/MS: (PS-A2) Rₜ 1.87 [M+H]⁺ 164.95.

### 36B. 4-(4-Chlorobenzoyl)piperidine-1-carboxylic acid tert-butyl ester

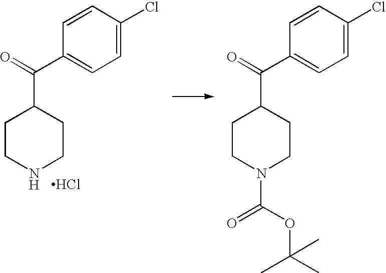

To a mixture of (4-chlorophenyl)piperidin-4-ylmethanone hydrochloride (0.996 g, 3.828 mmol) (Maybridge, CD10000) and triethylamine (2.7 ml, 19.142 mmol) in acetonitrile (15 ml) at room temperature was added di-tert-butyl dicarbonate (1.003 g, 4.594 mmol). After 16 hours at room temperature, the mixture was evaporated to dryness and then partitioned between ethyl acetate (50 ml) and 1M hydrochloric acid (20 ml). The organic phase was separated and washed successively with saturated aqueous sodium bicarbonate (20 ml), then brine (20 ml), before being dried over magnesium sulphate and concentrated to dryness. The crude material was purified by silica column chromatography (60% diethyl ether in hexanes) to give the ketone as an oil (1.116 g, 90%). LC/MS: (LCT1) Rₜ 7.42 [M+H]⁺ 323.

### 36C. 4-[Amino-(4-chlorophenyl)methyl]piperidine-1-carboxylic acid tert-butyl ester

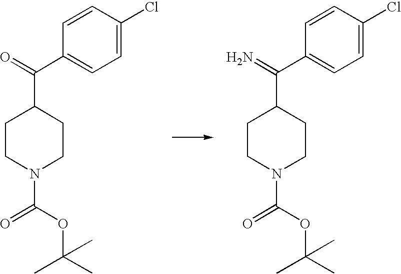

To a mixture of 4-(4-chlorobenzoyl)piperidine-1-carboxylic acid tert-butyl ester (1.116 g, 3.446 mmol) and ammonium acetate (3.188 g, 41.358 mmol) in methanol (34 ml) at room temperature was added sodium cyanoborohydride (0.866 g, 13.786 mmol). After refluxing for 20 hours, the mixture was cooled, concentrated and stirred with 1M sodium hydroxide (100 ml). The aqueous phase was extracted with diethyl ether (3×75 ml), with the organic layers being combined, dried over sodium sulphate and concentrated to dryness. The crude material was purified by silica column chromatography (15% methanol in DCM) to give the amine as an oil (0.913 g, 82%). LC/MS (LCT1): Rₜ 5.56 [M-Boc-NH₂]⁺ 208.

### 36D. 7-{4-[Amino-(4-chloro-phenyl)-methyl]-piperidin-1-yl}-3H-quinazolin-4-one

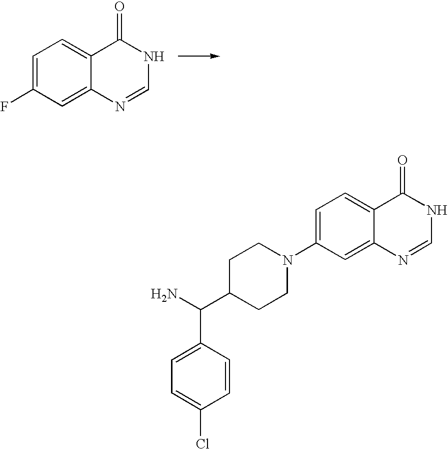

7-Fluoro-3H-quinazolin-4-one (0.038 g, 0.23 mmol) was reacted with 4-[amino-(4-chloro-phenyl)-methyl]-piperidine-1-carboxylic acid tert-butyl ester (0.150 g, 0.46 mmol) in water (1.0 ml) using the same procedure as described in Example 35E except that a power of 20 Watts was used in the microwave. The title compound was afforded as a white solid, 0.0474 g (56%). LC/MS: (PS-BE1) Rₜ 6.51 [M+H]⁺ 369.27. ¹H NMR (Me-d₃-OD) δ 1.12-1.35 (2H, m), 1.58-1.69 (1H, m), 1.88-1.97 (1H, m), 2.66-2.84 (2H, m), 3.58 (1H, d), 3.86-4.04 (2H, m), 6.87 (1H, s), 7.12 (1H, d), 7.32-7.39 (4H, m), 7.86 (1H, d), 7.92 (1H, s).

### Example 37

### 7-[4-(4-Chloro-phenyl)-piperidin-4-ylmethoxy]-1-methyl-1H-quinazoline-2,4-dione

### 37A. 4-Fluoro-2-methylamino-benzoic acid

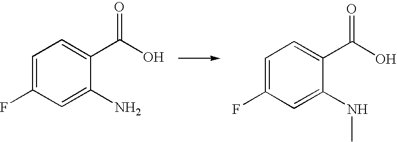

2-Amino-4-fluoro-benzoic acid (4.0 g, 25.79 mmol) was mixed with 10% palladium on carbon (1.0 g). The mixture was suspended in acetic acid (140 ml) and 37-40% w/v aqueous formaldehyde (13 ml) was added. The mixture was shaken under an atmosphere of hydrogen for 22 hours. The reaction mixture was then filtered through Celite, washing through with methanol and the filtrate was evaporated under reduced pressure. The residue was diluted with saturated aqueous sodium bicarbonate solution and this was extracted twice with ethyl acetate. The organics were dried (MgSO₄) and concentrated under reduced pressure. The residue was mixed with sodium hydroxide (9.27 g, 232 mmol) and then suspended in a mixture of tetrahydrofuran (105 ml) and water (105 ml). The suspension was heated at 70° C. for 2.5 hours and then allowed to cool to room temperature. The reaction mixture was evaporated under reduced pressure to remove the tetrahydrofuran. The residual aqueous layer was acidified to pH 7 with stirring using concentrated HCl. After stirring for 15 minutes, a precipitate was filtered off under reduced pressure, washed with water and dried to afford the title compound as a white solid, 2.015 g (46%). LC/MS: (PS-A2) Rₜ 2.71 [M+H]⁺ 170.16.

### 37B. 7-Fluoro-1-methyl-1H-quinazoline-2,4-dione

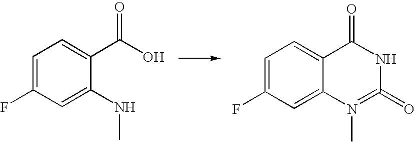

4-Fluoro-2-methylamino-benzoic acid (1.4 g, 8.28 mmol) was combined with urea (4.97 g, 82.8 mmol) and the mixture was heated at 160° C. with gentle stirring for 2 hours. The reaction mixture was then heated at 180° C. for 1.5 hours before allowing to cool to room temperature. The resulting solid was suspended in methanol and allowed to stand for 16 hours. The suspension was sonicated and diluted with dichloromethane and ethyl acetate and then evaporated down under reduced pressure. The residue was suspended in ethyl acetate and water and the undissolved solid was filtered off under reduced pressure, washing through with ethyl acetate and water. The biphasic filtrate was separated and the aqueous component was extracted twice with ethyl acetate. The organics were combined, dried (MgSO₄) and concentrated under reduced pressure. The residue was combined with the filtered solid from previously in the work up and purified by flash silica chromatography, eluting with diethyl ether to afford the title compound as a white solid, 0.14 g (9%). LC/MS: (PS-A2) Rₜ 2.21 [M+H]⁺ 195.16.

### 37C. 4-(4-Chloro-phenyl)-4-formyl-piperidine-1-carboxylic acid tert-butyl ester

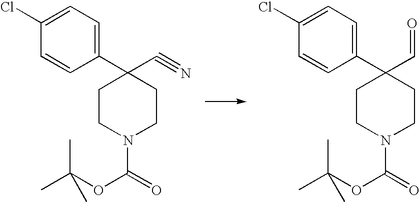

4-(4-Chloro-phenyl)-4-cyano-piperidine-1-carboxylic acid tert-butyl ester (4.34 g, 13.53 mmol) (Example 35C) was dissolved in anhydrous toluene (69 ml). The solution was cooled to −78° C. with stirring and a solution of 1M di-isobutyl-aluminium hydride in toluene (28.95 ml, 28.90 mmol) was added dropwise over 2 hours (temperature was maintained at −78° C.). The solution was allowed to warm to −35° C. over 2 hours and was stirred at −35° C. for a further 2 hours. Methanol (20 ml) was added dropwise followed by the dropwise addition of aqueous saturated ammonium chloride (20 ml). The reaction mixture solidified and was allowed to stand at room temperature for 18 hours before filtering under reduced pressure, washing through with ethyl acetate, dichloromethane and methanol. The filtrate was evaporated under reduced pressure until the aqueous layer remained. The aqueous was diluted with water and extracted twice with ethyl acetate. The organics were dried (MgSO₄) and concentrated under reduced pressure. The residue was purified by flash silica chromatography, eluting with a gradient of ethyl acetate/petroleum ether (5/95 to 50/50) to afford the title compound as a white solid, 1.565 g (36%). LC/MS: (PS-B4) Rₜ 3.61 [M+H]⁺ 324.17.

### 37D. 4-(4-Chloro-phenyl)-4-hydroxymethyl-piperidine-1-carboxylic acid tert-butyl ester

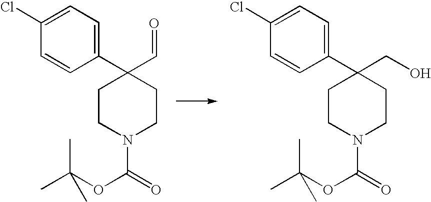

Sodium borohydride (0.066 g, 1.74 mmol) was dissolved in a mixture of ethanol (6.2 ml) and methanol (3 ml) with stirring. 4-(4-Chloro-phenyl)-4-formyl-piperidine-1-carboxylic acid tert-butyl ester (0.25 g, 0.772 mmol) was added slowly as a powder. The solution was stirred at room temperature for 2 hours. The reaction mixture was evaporated under reduced pressure, diluted with aqueous saturated sodium bicarbonate solution and extracted twice with ethyl acetate. The organics were dried (MgSO₄) and evaporated under reduced pressure to afford the title compound as a colourless oil, 0.256 g (100%). LC/MS: (PS-B3) Rₜ 3.32 [M+H]⁺ 326.31.

### 37E. 4-(4-Chloro-phenyl)-4-(1-methyl-2,4-dioxo-1,2,3,4-tetrahydro-quinazolin-7-yloxymethyl)-piperidine-1-carboxylic acid tert-butyl ester

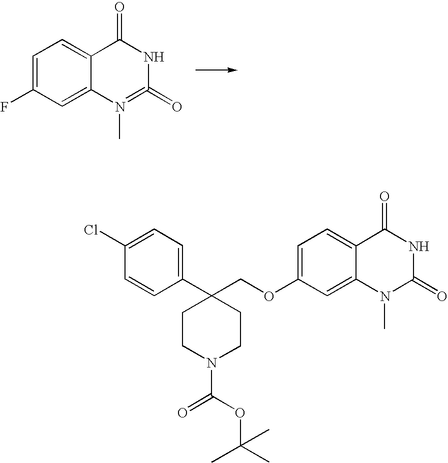

4-(4-Chloro-phenyl)-4-hydroxymethyl-piperidine-1-carboxylic acid tert-butyl ester (0.162 g, 0.497 mmol) was dissolved in anhydrous N,N-dimethylformamide (1.0 ml) in a ReactiVial™ (Pierce Chemical Co., Rockford, Ill.). The solution was cooled to 0° C. with stirring for 10 minutes and then sodium hydride (60% dispersion in oil, 0.0219 g, 0.547 mmol) was added. The resulting solution was warmed to room temperature and stirred for 1 hour. To this was added 7-fluoro-1-methyl-1H-quinazoline-2,4-dione (0.0241 g, 0.124 mmol) as a solid. The suspension was stirred at 140° C. under nitrogen for 2 hours. The reaction mixture was cooled to room temperature, diluted with water and extracted three times with ethyl acetate. The organic layer was dried (MgSO₄) and solvent was removed under reduced pressure. The residue was purified by flash silica chromatography, eluting with a gradient of ethyl acetate/petroleum ether (20/80 to 95/5) to afford the title compound as a colourless oil, 0.0403 g (65%). LC/MS: (PS-A2) Rₜ 3.50 [M+H]⁺ 500.22.

### 37F. 7-[4-(4-Chloro-phenyl)-piperidin-4-ylmethoxy]-1-methyl-1H-quinazoline-2,4-dione

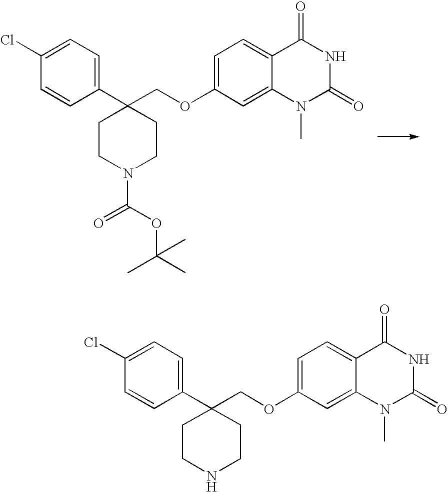

4-(4-Chloro-phenyl)-4-(1-methyl-2,4-dioxo-1,2,3,4-tetrahydro-quinazolin-7-yloxymethyl)-piperidine-1-carboxylic acid tert-butyl ester (0.0403 g, 0.0806 mmol) was dissolved in dichloromethane (5 ml) and 4M HCl in 1,4-dioxane (5 ml) was added. The solution was stirred at room temperature for 2 hours and then evaporated under reduced pressure. The residue was dissolved in methanol and eluted through a basic ion exchange column. The product was then purified by flash silica chromatography, eluting with 2M ammonia in methanol/dichloromethane (20/80). The product was further purified by preparative HPLC and then eluted through a basic ion exchange column. The product was dissolved in methanol and triturated by addition of diethyl ether. The triturated solid was filtered under reduced pressure, washed with diethyl ether and then dried to afford the title compound as a white solid, 0.0083 g (24%). LC/MS: (PS-BE1) Rₜ 6.02 [M+H]⁺ 400.24. ¹H NMR (Me-d₃-OD) δ 2.35 (2H, br t), 2.64 (2H, br d), 3.02 (2H, br t), 3.42 (2H, br d), 3.50 (3H, s), 4.17 (2H, s), 6.76 (1H, s), 6.83 (1H, d), 7.47 (2H, d), 7.59 (2H, d), 7.98 (1H, d).

### Example 38

### 7-[4-Amino-4-(4-chloro-benzyl)-piperidin-1-yl]-3H-quinazolin-4-one

### 38A. 4-(4-Chlorobenzyl)piperidine-1,4-dicarboxylic acid 1-tert-butyl ester 4-methyl ester

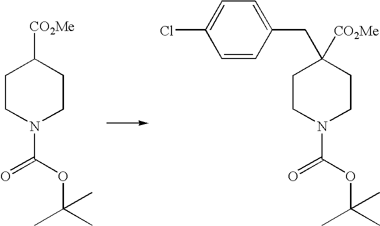

To a solution of isopropylamine (3.71 ml, 26.45 mmol) in THF (110 ml) at 0° C. was added n-butyllithium (10.1 ml of a 2.5M sol. in hexanes, 25.25 mmol). The resulting LDA solution was added via cannula to a solution of piperidine-1,4-dicarboxylic acid 1-tert-butyl ester 4-methyl ester* (5.85 g, 24.04 mmol) in THF (110 ml) and HMPA (20 ml) at −78° C. and stirring was continued for 1 hour. 4-Chlorobenzyl chloride (6.4 ml, 50.49 mmol) in THF (20 ml) was added and the solution was warmed to room temperature over 2 hours. After stirring for 18 hours, saturated aqueous ammonium chloride (500 ml) was added and the aqueous phase was extracted with diethyl ether (2×200 ml). The organic phases were combined, dried over magnesium sulphate and concentrated to dryness. Purification by silica column chromatography (0.5% methanol in DCM) gave the ester as an oil (3.03 g, 34%). LC/MS (LCT1): Rₜ 8.02 [M+Na⁺] 390.
This starting material can be made by the method described in Journal of Organic Chemistry (1990), 55(4), 1399-401.

### 38B. 4-(4-Chlorobenzyl)piperidine-1,4-dicarboxylic acid mono-tert-butyl ester

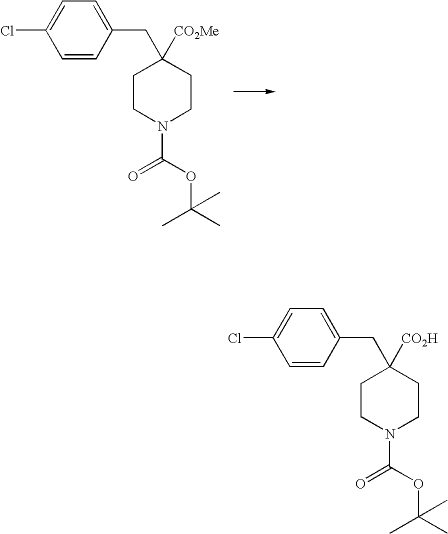

To a solution of 4-(4-chlorobenzyl)piperidine-1,4-dicarboxylic acid 1-tert-butyl ester 4-methyl ester (1.515 g, 4.117 mmol) in dioxane (20 ml), methanol (10 ml) and water (10 ml) at room temperature was added lithium hydroxide monohydrate (3.455 g, 82.341 mmol). After stirring at 50° C. for 2 days the solution was acidified to pH 6 with 2M HCl and the resulting white precipitate was extracted with diethyl ether (2×100 ml). The organic phases were combined, dried over sodium sulphate and concentrated to dryness, to give the acid as a white solid (1.460 g, 100%). LC/MS (LCT1): Rₜ 7.62 [M+Na⁺] 376.

### 38C. 4-(4-Chlorobenzyl)piperidin-4-yl amine dihydrochloride

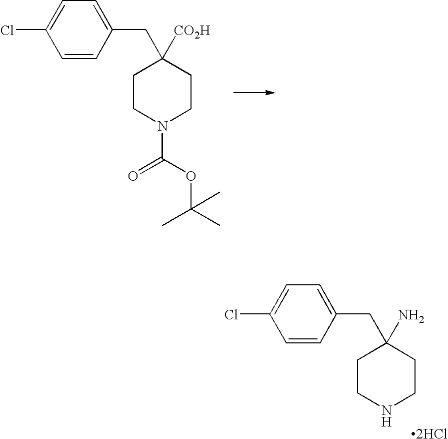

To a mixture of the acid (1.46 g, 4.126 mmol) and triethylamine (1.15 ml, 8.252 mmol) in THF (41 ml) at −15° C. was added isobutyl chloroformate (0.812 ml, 6.189 mmol). After 1 hour, a solution of sodium azide (0.536 g, 8.252 mmol) in water (10 ml) was added and the solution was warmed to room temperature overnight. Water (100 ml) was added and the aqueous phase was extracted with diethyl ether (3×50 ml). The organic phases were combined, washed with saturated sodium bicarbonate (50 ml) and dried over sodium sulphate. Toluene (100 ml) was added and the overall volume was reduced to approximately 90 ml. The resulting solution was warmed to 90° C. for 2 hours, then cooled and added to 10% hydrochloric acid (70 ml). The biphasic mixture was warmed to 90° C. for 24 h. The organic phase was separated and concentrated to dryness to give the crude amine salt (1.109 g). The crude amine salt was dissolved in 2M NaOH (20 ml) and di-tert-butyl dicarbonate (1.61 g, 7.391 mmol) added. After 2 days the aqueous phase was extracted with diethyl ether (2×50 ml). The organic phases were combined, washed with 1M HCl (20 ml), saturated sodium bicarbonate (20 ml) and brine (20 ml), then dried over magnesium sulphate and concentrated. Purification by column chromatography (50% diethyl ether in hexanes) gave the doubly BOC-protected amine (0.685 g), which was subsequently deprotected by stirring with 4M HCl in dioxane (10 ml) and methanol (10 ml) at r.t. for 2 days. Concentration gave the desired amine as the bis-hydrochloride salt (0.492 g, 40% from acid). ¹H NMR (Me-d₃-OD) δ 2.18-2.13 (4H, m), 3.21 (2H, s), 3.53-3.47 (4H, m), 7.35-7.32 (2H, m), 7.48-7.44 (2H, m)

### 38D. 7-[4-Amino-4-(4-chloro-benzyl)-piperidin-1-yl]-2-methyl-3H-quinazolin-4-one

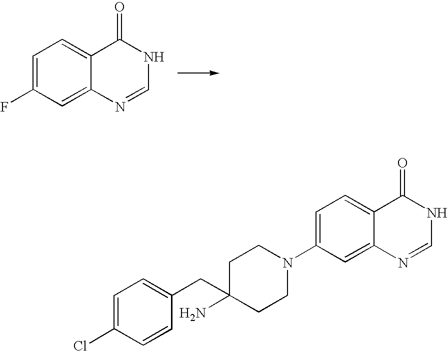

7-Fluoro-3H-quinazolin-4-one (0.0193 g, 0.118 mmol) and 4-(4-chloro-benzyl)-piperidin-4-ylamine dihydrochloride (0.035 g, 0.118 mmol) were both weighed into a ReactiVial™ (Pierce Chemical Co., Rockford, Ill.). The mixture was suspended in 1-butanol (1.18 ml) and triethylamine (0.0805 ml, 0.59 mmol) was added. The reaction was sealed and heated at 175° C. for 3 hours. The reaction mixture was allowed to cool to room temperature, diluted with ethyl acetate and washed twice with water. The aqueous was then extracted once with ethyl acetate and the organics were combined, dried (MgSO₄) and evaporated under reduced pressure.

The product was purified by flash silica chromatography, eluting with methanol/ethyl acetate (20/80) followed by preparative HPLC. The product was then eluted through a basic ion exchange column to afford the title compound as a colourless oil, 0.0032 g (7%). LC/MS: (PS-BE1) Rₜ 6.29 [M+H]⁺ 369.27. ¹H NMR (Me-d₃-OD) δ 1.52-1.62 (2H, m), 1.73-1.85 (2H, m), 2.79 (2H, s), 3.40-3.50 (2H, m), 3.65-3.75 (2H, m), 6.99 (1H, s), 7.20-7.30 (3H, m), 7.34 (2H, d), 7.99-8.06 (2H, m).

### Example 39

### 7-{2-[4-(4-Chloro-phenyl)-piperidin-4-yl]-vinyl}-3H-quinazolin-4-one dihydrochloride

### 39A. 2-Amino-4-bromo-benzoic acid

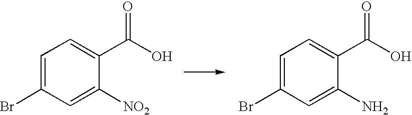

4-Bromo-2-nitro-benzoic acid (0.5 g, 2.03 mmol) (Matrix, 009241) was dissolved in a 1:1 mixture of ethanol/tetrahydrofuran (22 ml). This solution was added to 5% platinum on carbon (0.2 g, 50% water content) under an atmosphere of nitrogen. The reaction was shaken under an atmosphere of hydrogen for 2.5 hours. A further batch of platinum on carbon was added (0.2 g) and the mixture was shaken for 64 hours under an atmosphere of hydrogen. The reaction mixture was filtered, washing through with a 1:1 mixture of ethanol/tetrahydrofuran. The solvent was removed under reduced pressure and the residue was purified by flash silica chromatography, eluting with methanol/dichloromethane (2/98) to yield the title compound as a yellow solid (0.253 g, 58%). LC/MS: (PS-A1) Rₜ 2.62 [M+H]⁺ 215.88.

### 39B. 7-Bromo-3H-quinazolin-4-one

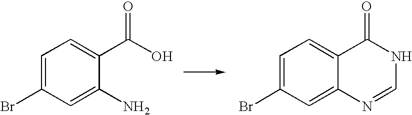

2-Amino-4-bromo-benzoic acid (0.5 g, 2.31 mmol) was converted to 7-bromo-3H-quinazolin-4-one using the same procedure as described for Example 36A to yield the title compound as a beige solid (0.285 g, 55% yield). LC/MS: (PS-A2) Rₜ 2.20 [M+H]⁺ 224.8

### 39C. 4-(4-Chloro-phenyl)-4-vinyl-piperidine-1-carboxylic acid tert-butyl ester

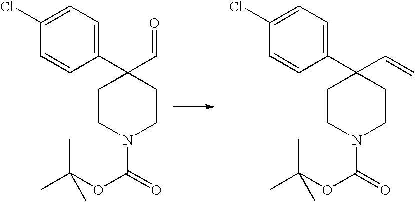

Methyltriphenylphosphonium iodide (2.74 g, 6.79 mmol) was suspended in anhydrous tetrahydrofuran (70 ml) and cooled to −10° C. under nitrogen. A 1.6M solution of butyl lithium in hexanes (4.24 ml, 6.79 mmol) was added dropwise. The solution was stirred at −10° C. for 40 minutes and was then cooled to −78° C. 4-(4-Chloro-phenyl)-4-formyl-piperidine-1-carboxylic acid tert-butyl ester (1.565 g, 4.83 mmol) (see Example 37C) was dissolved in anhydrous tetrahydrofuran (35 ml) and this solution was added dropwise. The reaction mixture was stirred for 18 hours during which time the reaction warmed to room temperature. Water (5 ml) was added and the reaction mixture was evaporated under reduced pressure. The residue was diluted with water and extracted three times with ethyl acetate. The organics were dried (MgSO₄) and concentrated under reduced pressure. The product was purified by flash silica chromatography, eluting with a gradient of ethyl acetate/petroleum ether (3/97 to 30/70) to afford the title compound as a colourless oil, 1.32 g (85%). LC/MS: (PS-A2) Rₜ 4.10 [M+H−tert-butyl]⁺ 266.06.

### 39D. 4-(4-Chloro-phenyl)-4-[2-(4-oxo-3,4-dihydro-quinazolin-7-yl)-vinyl]-piperidine-1-carboxylic acid tert-butyl ester

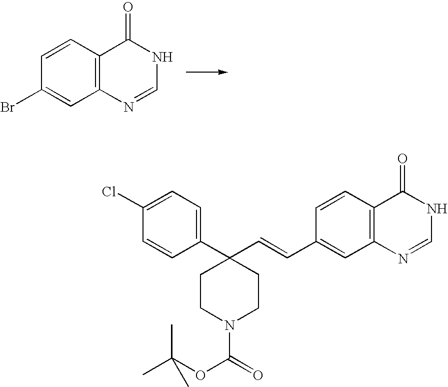

7-bromo-3H-quinazolin-4-one (0.923 g, 4.10 mmol), 4-(4-chloro-phenyl)-4-vinyl-piperidine-1-carboxylic acid tert-butyl ester (1.32 g, 4.10 mmol) and tetraethylammonium chloride (0.679 g, 4.10 mmol) were combined as solids in a Schlenk tube and suspended in anhydrous N-methylpyrrolidinone (9.23 ml). Dicyclohexylmethylamine (1.32 ml, 6.15 mmol) was added and the mixture was degassed with nitrogen. Palladium (II) acetate (0.046 g, 0.205 mmol) was added and the reaction was heated at 125° C. for 2 hours under nitrogen. The reaction mixture was allowed to cool to room temperature and was then diluted with water. The aqueous was extracted three times with ethyl acetate. The organics were dried (MgSO₄) and concentrated under reduced pressure. The residue was purified by flash silica chromatography, eluting with a gradient of methanol/ethyl acetate (1/99 to 10/90) to afford the title compound as a yellow foam, 0.759 g (40%). LC/MS: (PS-A2) Rₜ 3.47 [M+H]⁺ 466.15.

### 39E. 7-{2-[4-(4-Chloro-phenyl)-piperidin-4-yl]-vinyl}-3H-quinazolin-4-one

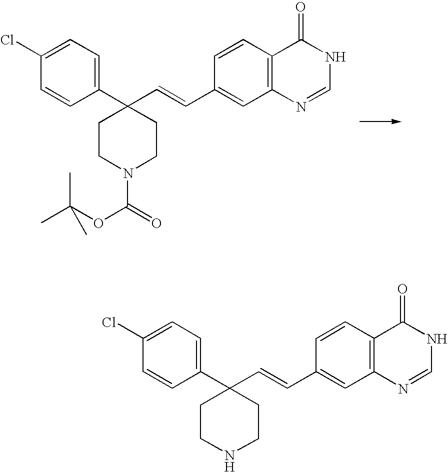

4-(4-Chloro-phenyl)-4-[2-(4-oxo-3,4-dihydro-quinazolin-7-yl)-vinyl]-piperidine-1-carboxylic acid tert-butyl ester (0.75 g, 1.61 mmol) was dissolved in dichloromethane (10 ml) and saturated HCl in diethyl ether (10 ml) was added. Stirring was continued for 2 hours. The reaction mixture was evaporated under reduced pressure and the residue was eluted through a basic ion exchange column. The product was purified by flash silica chromatography, eluting with 2M ammonia in methanol/dichloromethane (20/80). The product was triturated with a mixture of diethyl ether/petroleum ether (50/50). The triturated product was filtered and dried to afford the title compound as a white solid, 0.395 g (67%). LC/MS: (PS-BE2) Rₜ 5.74 [M+H]⁺ 366.17.

### 39F. 7-{2-[4-(4-Chloro-phenyl)-piperidin-4-yl]-vinyl}-3H-quinazolin-4-one dihydrochloride

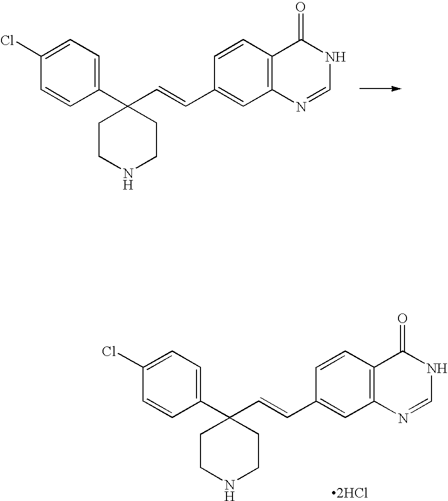

7-{2-[4-(4-Chloro-phenyl)-piperidin-4-yl]-vinyl}-3H-quinazolin-4-one (0.144 g, 0.393 mmol) was dissolved in 2M aqueous HCl (10 ml). The solution was stirred at room temperature for 2 hours and then concentrated under reduced pressure to afford the title compound as a pale blue solid, 0.176 g (100%). LC/MS: (PS-BE2) Rₜ 5.70 [M+H]⁺ 366.11. ¹H NMR (Me-d₃-OD) δ 2.47-2.56 (4H, m), 3.21-3.41 (4H, m), 6.67 (1H, d), 6.79 (1H, d), 7.44 (2H, d), 7.50 (2H, d), 7.77 (1H, s), 7.90 (1H, d), 8.26 (1H, d), 9.26 (1H, s).

### Example 40

### 7-[4-Amino-4-(4-chloro-phenyl)-piperidin-1-yl]-3H-quinazolin-4-one

### 40A. 4-(4-Chloro-phenyl)-piperidine-1,4-dicarboxylic acid mono-tert-butyl ester

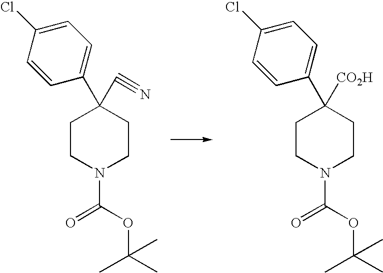

A solution of 4-(4-chlorophenyl)-4-cyanopiperidine-1-carboxylic acid tert-butyl ester (0.683 g, 2.129 mmol) in 6M HCl (20 ml) was refluxed for 4 days. The solution was cooled, basified with NaOH and di-tert-butyl dicarbonate (0.558 g, 2.555 mmol) was added. After stirring for 24 hours, the solution was extracted with diethyl ether (2×75 ml). The organic phases were combined, washed with brine (50 ml), dried over magnesium sulphate and concentrated. Purification by silica column chromatography (5% methanol in DCM) gave the acid as a white foam (0.339 g, 47%). LC/MS (LCT2): Rₜ 8.17 [M+Na⁺] 362.

### 40B. 4-(4-Chlorophenyl)piperidin-4-yl amine dihydrochloride

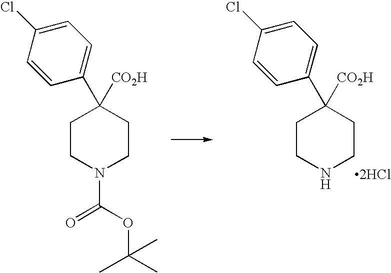

The title compound was prepared using the method described for Example 38C. ¹H NMR (Me-d₃-OD) δ 2.56-2.44 (2H, m), 3.07-2.93 (4H, m), 3.61-3.52 (2H, m), 7.65-7.61 (2H, m), 7.74-7.70 (2H, m).

### 40C. 7-[4-Amino-4-(4-chloro-phenyl)-piperidin-1-yl]-3H-quinazolin-4-one

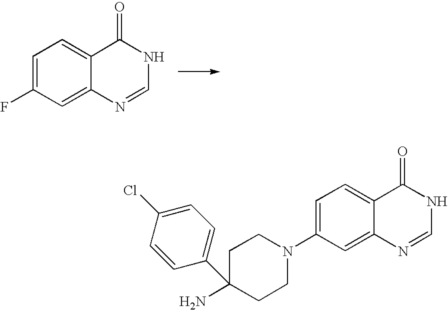

7-Fluoro-3H-quinazolin-4-one (0.0173 g, 0.106 mmol) was reacted with 4-(4-chloro-phenyl)-piperidin-4-ylamine dihydrochloride (0.030 g, 0.106 mmol) using the same procedure as described in Example 38D except that after heating for 30 minutes at 175° C., an additional quantity of triethylamine (0.075 ml, 0.55 mmol) and 1-butanol (1 ml) was added. After heating for a further 4.5 hours, an additional quantity of triethylamine (0.075 ml, 0.55 mmol) was added. Heating was then continued for a further 15 hours. The reaction mixture was allowed to cool to room temperature and was diluted with water and extracted twice with ethyl acetate. The organics were dried (MgSO₄) and concentrated under reduced pressure. The residue was purified by preparative HPLC followed by elution through a basic ion exchange column to afford the title compound as a colourless gum, 0.0042 g (11%). LC/MS: (PS-BE1) Rₜ 5.95 [M+H]⁺ 355.13. ¹H NMR (Me-d₃-OD) δ 1.76-1.85 (2H, m), 2.12-2.23 (2H, m), 3.43-3.59 (4H, m), 6.92 (1H, s), 7.15 (1H, d), 7.26 (2H, d), 7.44 (2H, d), 7.88 (1H, s), 7.92 (1H, d).

### Example 41

### 7-[4-Aminomethyl-4-(4-chloro-benzyl)-piperidin-1-yl]-3H-quinazolin-4-one

### 41A. 4-(4-Chlorobenzyl)-4-cyanopiperidine-1-carboxylic acid tert-butyl ester

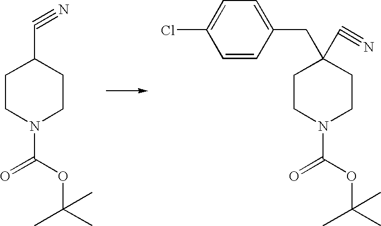

To a solution of isopropylamine (1.53 ml, 10.94 mmol) in THF (30 ml) at −78° C. was added n-butyllithium (4.38 ml of a 2.5M solution in hexanes, 10.938 mmol). After 10 minutes, a solution of 4-cyanopiperidine-1-carboxylic acid tert-butyl ester* in THF (12 ml) was added. After a further 1 hour, a solution of 4-chlorobenzyl chloride (1.84 g, 11.4 mmol) in THF (5 ml) was added and the solution warmed to room temperature over 15 hours. Water (150 ml) was added and the aqueous phase extracted with diethyl ether (150 ml). The organic phase was dried over magnesium sulphate and concentrated to give a crude solid that was purified by recrystallisation from diethyl ether/hexane in two batches to give the product as a white solid (2.650 g, 83%). LC/MS (LCT2): Rₜ 8.02 [M+Na⁺] 357, [M-Boc]⁺ 235.
This starting material was made by the method described in Chem. Pharm. Bull., 2001, 49(7), 822-829.

### 41B. C-[4-(4-Chlorobenzyl)piperidin-4-yl]methyl amine dihydrochloride

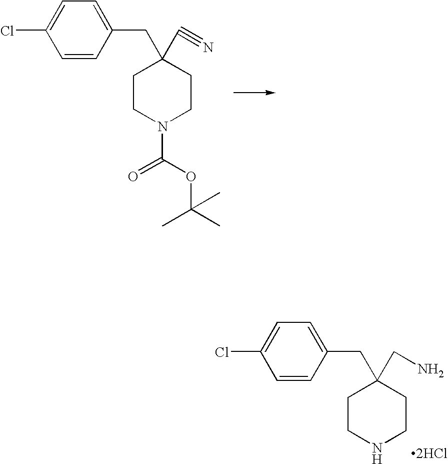

To a solution of 4-(4-chlorobenzyl)-4-cyanopiperidine-1-carboxylic acid tert-butyl ester (0.500 g, 1.493 mmol) in methanol (3 ml) was added 4M HCl in dioxane (10 ml). After stirring for 19 hours, the solution was concentrated to give the deprotected amine as the hydrochloride salt (0.405 g).

The amine salt was dissolved in 1M BH₃.THF in THF (15 ml, 15 mmol) at room temperature and stirred for 2 days. The reaction was quenched with methanol (10 ml), concentrated, redissolved in methanol (10 ml) and 4M HCl in dioxane (20 ml) and the resulting solution refluxed for 6 hours. Concentration and purification by SCX-2 Isolute column (5 g), eluting with 1M NH₃/MeOH, gave the desired amine, which was converted to the bis-hydrochloride salt by dissolving in 2M aqueous HCl (6 ml) and methanol (6 ml) followed by concentration to give the product as a white solid (0.285 g, 61%). ¹H NMR (Me-d₃-OD)-free amine-δ 1.45-1.41 (4H, m), 2.52 (2H, s), 2.70 (2H, s), 2.94-2.75 (4H, m), 7.20-7.17 (2H, m), 7.31-7.28 (2H, m).

### 41C. 7-[4-Aminomethyl-4-(4-chloro-benzyl)-piperidin-1-yl]-3H-quinazolin-4-one

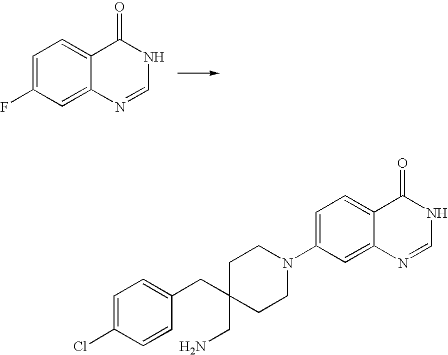

7-Fluoro-3H-quinazolin-4-one (0.0184 g, 0.112 mmol) was reacted with C-[4-(4-chloro-benzyl)-piperidin-4-yl]-methylamine dihydrochloride (0.035 g, 0.112 mmol) using the same procedure as described in Example 40C except that heating was continued for a total of 26 hours. The title compound was afforded as a colourless gum, 0.00128 g (3%). LC/MS: (PS-BE2) Rₜ 6.37 [M+H]⁺ 383. ¹H NMR (Me-d₃-OD) δ 1.52 (4H, t), 2.46 (2H, s), 2.66 (2H, s), 3.29-3.53 (4H, m), 6.86 (1H, s), 7.06-7.14 (3H, m), 7.19 (2H, d), 7.88 (1H, s), 7.91 (1H, d).

### Example 42

### 7-[4-Aminomethyl-4-(4-chloro-benzyl)-piperidin-1-yl]-1-methyl-1H-quinazoline-2,4-dione

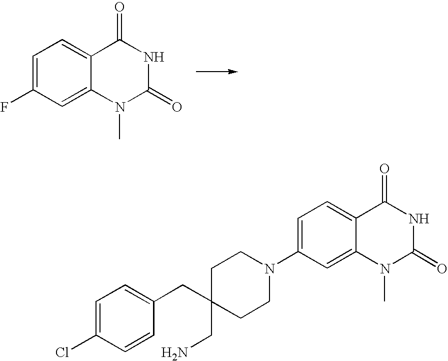

### 42A. 7-Fluoro-1-methyl-1H-quinazoline-2,4-dione

The title compound was prepared using the methods described in Example 37A and Example 37B.

### 42B. 7-[4-Aminomethyl-4-(4-chloro-benzyl)-piperidin-1-yl]-1-methyl-1H-quinazoline-2,4-dione

The title compound was prepared using the method described in Example 27 except that 7-fluoro-1-methyl-1H-quinazoline-2,4-dione was used instead of 7-fluoro-3H-quinazolin-4-one. LC-MS PS-BE1 Rₜ 6.39 [M+H]⁺ 399.28.

¹H NMR (d6-DMSO) δ 7.74 (1H, d), 7.41 (4H, m), 6.83 (1H, br d), 6.52 (1H, br s), 3.73 (2H, m), 3.41 (3H, s), 3.12-3.01 (2H, m), 2.66 (2H, s), 2.23-2.13 (2H, m), 1.94-1.83 (2H, m).

### Biological Activity

### Example 43

### Measurement of PKA Kinase Inhibitory Activity (IC₅₀)

Compounds of the invention can be tested for PK inhibitory activity using the PKA catalytic domain from Upstate Biotechnology (#14-440) and the 9 residue PKA specific peptide (GRTGRRNSI), also from Upstate Biotechnology (#12-257), as the substrate. A final concentration of 1 nM enzyme is used in a buffer that includes 20 mM MOPS pH 7.2, 40 μM ATP/γ³³P-ATP and 50 mM substrate. Compounds are added in dimethylsulphoxide (DMSO) solution to a final DMSO concentration of 2.5%. The reaction is allowed to proceed for 20 minutes before addition of excess orthophosphoric acid to quench activity. Unincorporated γ³³P-ATP is then separated from phosphorylated proteins on a Millipore MAPH filter plate. The plates are washed, scintillant is added and the plates are then subjected to counting on a Packard Topcount.

The % inhibition of the PKA activity is calculated and plotted in order to determine the concentration of test compound required to inhibit 50% of the PKA activity (IC₅₀).

### Example 44

### Measurement of PKB Kinase Inhibitory Activity (IC₅₀)

The inhibition of protein kinase B (PKB) activity by compounds can be determined essentially as described by Andjelkovic et al. (Mol. Cell. Biol. 19, 5061-5072 (1999)) but using a fusion protein described as PKB-PIF and described in full by Yang et al (Nature Structural Biology 9, 940-944 (2002)). The protein is purified and activated with PDK1 as described by Yang et al. The peptide AKTide-2T (H-A-R-K-R-E-R-T-Y-S-F-G-H-H-A-OH) obtained from Calbiochem (#123900) is used as a substrate. A final concentration of 0.6 nM enzyme is used in a buffer that includes 20 mM MOPS pH 7.2, 30 μM ATP/γ³³P-ATP and 25 μM substrate. Compounds are added in DMSO solution to a final DMSO concentration of 2.5%. The reaction is allowed to proceed for 20 minutes before addition of excess orthophosphoric acid to quench activity. The reaction mixture is transferred to a phosphocellulose filter plate where the peptide binds and the unused ATP is washed away. After washing, scintillant is added and the incorporated activity measured by scintillation counting.

The % inhibition of the PKB activity is calculated and plotted in order to determine the concentration of test compound required to inhibit 50% of the PKB activity (IC₅₀).

Following the protocol described above, the IC₅₀ values of the compounds of Examples 1 to 9, 14 to 22 and 27 to 42 have been found to be less than 10 μM whilst the compounds of Examples 10 to 13, 23, 25 and 26 each have IC₅₀ values of less than 50 μM.

### Example 45

### Anti-Proliferative Activity

The anti-proliferative activities of compounds of the invention are determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines are obtained from ECACC (European Collection of cell Cultures) or ATCC.

In particular, compounds of the invention were tested against the PC3 cell line (ATCC Reference: CRL-1435) derived from human prostate adenocarcinoma. Many compounds of the invention were found to have IC₅₀ values of less than 50 μM in this assay and preferred compounds have IC₅₀ values of less than 15 μM.

### Pharmaceutical Formulations

### Example 46

### (i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (I) with 100 mg lactose and filling the resulting mixture into standard opaque hard gelatin capsules.

### (iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

### (iv) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1.-78. (canceled)

79. A method for the prophylaxis or treatment of a disease or condition comprising or arising from abnormal cell growth or abnormally arrested cell death in a mammal, which method comprises administering to the mammal an effective amount of a compound of the formula (I): 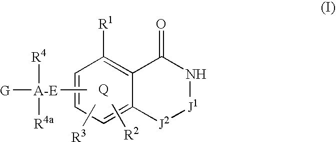
or a salt, solvate, tautomer or N-oxide thereof, wherein:
the ring Q is a benzene ring;
J²-J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷; provided that when J²-J¹ is a group R^{1a}N—CO, E is other than NR⁷CO;
m and n are each 0 or 1, provided that the sum of m and n is 1 or 2;
A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; NH₂; NHCOR¹⁰ and NHCONHR¹⁰;
R⁴ is hydrogen or C₁₋₄ alkyl;
R^{4a} is hydrogen, C₁₋₄ alkyl or a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N;
R¹⁰ is phenyl or benzyl each optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

80. A method according to claim 79 wherein the compound is a compound of the formula (I⁰): 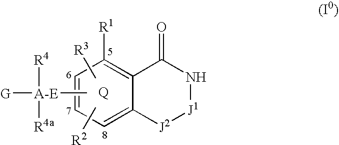
or a salt, solvate, tautomer or N-oxide thereof, wherein:
the ring Q is a benzene ring;
J² J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷; provided that when J²-J¹ is a group R^{1a}N—CO, E is other than NR⁷CO;
m and n are each 0 or 1, provided that the sum of m and n is 1 or 2;
A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; NH₂; NHCOR¹⁰ and NHCONHR¹⁰;
R⁴ is hydrogen or C₁₋₄ alkyl;
R^{4a} is hydrogen, C₁₋₄ alkyl or a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N;
R¹⁰ is phenyl or benzyl each optionally substituted by one or more substituents selected from halogen, hydroxy, trifluoromethyl, cyano, nitro, carboxy, amino, mono- or di-C₁₋₄ hydrocarbylamino; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
and provided that when A is a bond, G and E combine to form a group R⁶R⁵NC(O)NH— attached to the ring Q at the position marked with the numeral 7;
and that when G is OH, A is other than a bond and R^{4a} is R⁹.

81. A compound of the formula (Ia): 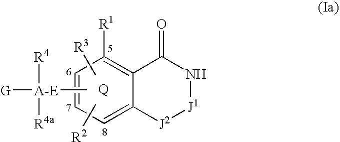
or salts, solvates, tautomers or N-oxides thereof, wherein:
the ring Q is a benzene ring;
J²-J¹ is a group N═CR⁷ or a group R^{1a}N—CO;
G is OH or NR⁵R⁶;
E is a linking atom or group selected from CONR⁷, NR⁷CO, C(R⁸)═C(R⁸), (X)ₘ(CR⁸R^{8a})ₙ where X is selected from O, S and NR⁷, whereby when J²-J¹ is a group R^{1a}N—CO, E is other than NR⁷CO;
m and n are each 0 or 1, provided that the sum of m and n is 1 or 2;
A is a bond and R⁴ and R^{4a} are absent, or A is a saturated hydrocarbon linker group containing from 1 to 7 carbon atoms, the linker group having a maximum chain length of 5 atoms extending between E and G, wherein one of the carbon atoms in the linker group A may optionally be replaced by an oxygen or nitrogen atom; and wherein the carbon atoms of the linker group A may optionally bear one or more substituents selected from oxo, fluorine and hydroxy, provided that the hydroxy group and oxo group when present are not located at a carbon atom a with respect to the group G;
the moiety A-E having a minimum chain length of 2 atoms extending between the ring Q and the nitrogen or oxygen atom of the group G;
R¹, R^{1a}, R², and R³ are each independently selected from hydrogen; halogen; C₁₋₆ hydrocarbyl optionally substituted by halogen, hydroxy or C₁₋₂ alkoxy; cyano; CONHR⁸; and NH₂; provided that when A is a bond and E is CONR⁷, R² is attached to the carbon atom designated by the numeral 8 on the benzene ring Q;
R⁴ is hydrogen or C₁₋₄ alkyl;
R^{4a} is a group R⁹;
R⁵ and R⁶ are each selected from hydrogen, a group R⁹ and C₁₋₄ hydrocarbyl optionally substituted by halogen or C₁₋₂ alkoxy or by a group R⁹; or NR⁵R⁶ forms a saturated monocyclic heterocyclic group having 4-7 ring members and optionally containing a second heteroatom ring member selected from O and N;
R⁷ is selected from hydrogen and C₁₋₄ alkyl;
R⁸ and R^{8a} are selected from hydrogen and saturated C₁₋₄ hydrocarbyl optionally substituted by one or more fluorine atoms;
R⁹ is a monocyclic or bicyclic carbocyclic or heterocyclic group containing up to 3 ring heteroatoms selected from N, O and S;
or R⁴ and R^{4a} together with the intervening atom or atoms of the group A form a saturated monocyclic heterocyclic group having 4-7 ring members and optionally containing a second heteroatom ring member selected from O and N;
or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
or R⁴ together with R⁷ or R⁸ and the intervening atoms of the groups A and E form a saturated monocyclic heterocyclic group having 4-7 ring members and optionally containing a second heteroatom ring member selected from O and N;
or one of R⁵ and R⁶ together with the nitrogen atom to which they are attached and R⁷ or R⁸ and the intervening atoms of the groups A and E form a saturated monocyclic heterocyclic group having 4-7 ring members and optionally containing a second heteroatom ring member selected from O and N;
and provided that:
(a) when J²-J¹ is a group R^{1a}N—CO, E is a linking atom or group E′ selected from CH═CH, (X′)ₘ(CH₂)ₙ where X is selected from O and S; and/or one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N;
(b) when A is a bond, G and E combine to form a group R⁶R⁵NC(O)NH— attached to the ring Q at the position marked with the numeral 7, wherein at least one of R⁵ and R⁶ is other than hydrogen;
(c) when R⁴ together with R⁷ and the intervening atoms of the groups A and E form a piperidine ring and G is NR⁵R⁶ attached directly to the 3-position of the piperidine ring, then R^{4a} is other than cycloalkyl;
(d) when J²-J¹ is a group N═C(Me), the moiety R⁶R⁶N-A(R⁴)(R^{4a})-E- is other than a 2-phenyl-3-hydroxypropyl group attached to the ring Q at the carbon atom marked by the numeral 6;
(e) when G is OH and J²-J¹ is a group N═CR⁷, then R⁷ is other than an alkyl group having three or more carbon atoms;
(f) when one of R⁵ and R⁶ together with the nitrogen atom to which they are attached and R⁷ and the intervening atoms of the groups A and E form a saturated monocyclic heterocyclic group, then J²-J¹ is other than a group HN—CO;
(g) when E is (X)ₘ(CR⁸R^{8a})ₙ, m is 0 and n is 1; then J²-J¹ is other than a group HN—CO; and
(h) when the moiety R⁶R⁵N-A(R⁴)(R^{4a})-E- is a 2-morpholinoethoxy group, then J²-J¹ is other than a group HN—CO.

82. A compound according to claim 81 wherein J²J¹ is a group N═CH or a group R^{1a}N—CO wherein R^{1a} is hydrogen or C₁₋₄ alkyl.

83. A compound according to claim 81 wherein A is a saturated hydrocarbon linker group containing from 1 to 7 carbon atoms, the linker group having a maximum chain length of 5 atoms extending between E and G.

84. A compound according to claim 81 wherein R^{4a} is a group R⁹ and the linker group has a maximum chain length of 4 atoms extending between R⁹ and G.

85. A compound according to claim 84 wherein the linker group A has a chain length of 3 atoms extending between R⁹ and G and a chain length of 3 or 4 atoms extending between E and G.

86. A compound according to claim 81 wherein G is NR⁵R⁶ and R⁵ and R⁶ are independently selected from hydrogen and saturated C₁₋₄ hydrocarbyl.

87. A compound according to claim 86 wherein R⁵ and R⁶ are independently selected from hydrogen and methyl.

88. A compound according to claim 81 wherein one of R⁵ and R⁶ together with the nitrogen atom to which they are attached and R⁴ and one or more atoms from the linker group A form a saturated monocyclic heterocyclic group having 4-7 ring members and optionally containing a second heteroatom ring member selected from O and N.

89. A compound according to claim 88 wherein the moiety E-A(R⁴)(R^{4a})-G is selected from: 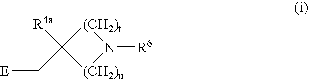
where t and u are each 0, 1, 2 or 3 provided that the sum of t and u falls within the range of 2 to 4; and 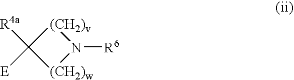
where v and w are each 0, 1, 2 or 3 provided that the sum of v and w falls within the range of 2 to 5.

90. A compound according to claim 89 wherein R^{4a} is a group R⁹.

91. A compound according to claim 81 wherein R⁴ together with R⁷ or R⁸ and the intervening atoms of the groups A and E form a saturated monocyclic heterocyclic group having 4-7 ring members and optionally containing a second heteroatom ring member selected from O and N.

92. A compound according to claim 81 wherein R⁴ is hydrogen.

93. A compound according to claim 81 wherein R⁹ is a monocyclic aryl or heteroaryl group which is unsubstituted or substituted up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy; C₁₋₄ acylamino; benzoylamino; pyrrolidinocarbonyl; piperidinocarbonyl; morpholinocarbonyl; piperazinocarbonyl; five and six membered heteroaryl groups containing one or two heteroatoms selected from N, O and S, the heteroaryl groups being optionally substituted by one or more C₁₋₄ alkyl substituents; phenyl; pyridyl; and phenoxy wherein the phenyl, pyridyl and phenoxy groups are each optionally substituted with 1, 2 or 3 substituents selected from C₁-C₂ acyloxy, fluorine, chlorine, bromine, trifluoromethyl, cyano, C₁₋₂ hydrocarbyloxy and C₁₋₂ hydrocarbyl each optionally substituted by methoxy or hydroxy.

94. A compound according to claim 93 wherein the aryl group is an optionally substituted phenyl group.

95. A compound according to claim 94 wherein the phenyl group is unsubstituted or substituted by up to 5 substituents selected from hydroxy; C₁₋₄ acyloxy; fluorine; chlorine; bromine; trifluoromethyl; cyano; C₁₋₄ hydrocarbyloxy and C₁₋₄ hydrocarbyl each optionally substituted by C₁₋₂ alkoxy or hydroxy.

96. A compound according to claim 95 wherein the phenyl group has one or two substituents selected from fluorine, chlorine, trifluoromethyl, methyl and methoxy.

97. A compound according to claim 96 wherein the phenyl group is selected from mono-chlorophenyl and dichlorophenyl.

98. A compound according to claim 81 wherein E is selected from CONR⁷ and NR⁷CO wherein R⁷ is hydrogen.

99. A compound according to claim 81 wherein R¹, R² and R³ each are hydrogen.

100. A compound according to claim 81 having the formula (II): 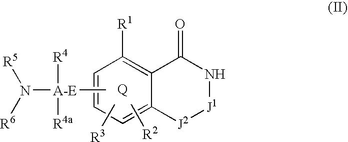
or salts, solvates, tautomers or N-oxides thereof; wherein R¹ to R⁶, A, E, J¹ and J² are as defined in claim 3.

101. A compound according to claim 100 of the formula (III): 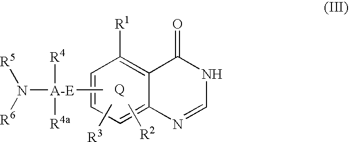
or salts, solvates, tautomers or N-oxides thereof.

102. A compound according to claim 101 of the formula (IV): 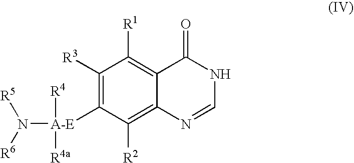
or salts, solvates, tautomers or N-oxides thereof.

103. A compound according to claim 81 selected from the group consisting of:
4-amino-2-(3,4-dichloro-phenyl)-N-(4-oxo-3,4-dihydro-quinazolin-7-yl)-butyramide;
2-(4-chloro-phenyl)-4-methylamino-N-(4-oxo-3,4-dihydro-quinazolin-7-yl)-butyramide;
4-oxo-3,4-dihydro-quinazoline-7-carboxylic acid[3-amino-1-(4-chloro-phenyl)-propyl]-amide;
4-phenyl-piperidine-4-carboxylic acid (4-oxo-3,4-dihydro-quinazolin-7-yl)-amide;
7-[4-aminomethyl-4-(4-chloro-phenyl)-piperidin-1-yl]-3H-quinazolin-4-one;
(S)-4-amino-2-(3,4-dichloro-phenyl)-N-(4-oxo-3,4-dihydro-quinazolin-7-yl)butyramide;
(R)-4-amino-2-(3,4-dichloro-phenyl)-N-(4-oxo-3,4-dihydro-quinazolin-7-yl)butyramide;
4-(4-chloro-phenyl)-piperidine-4-carboxylic acid (4-oxo-3,4-dihydro-quinazolin-7-yl)-amide;
7-[4-aminomethyl-4-(4-chloro-phenyl)-piperidin-1-yl]-2-methyl-3H-quinazolin-4-one;
7-{4-[amino-(4-chloro-phenyl)-methyl]-piperidin-1-yl}-3H-quinazolin-4-one;
7-[4-(4-chloro-phenyl)-piperidin-4-ylmethoxy]-1-methyl-1H-quinazoline-2,4-dione;
7-[4-amino-4-(4-chloro-benzyl)-piperidin-1-yl]-3H-quinazolin-4-one;
7-{2-[4-(4-chloro-phenyl)-piperidin-4-yl]-vinyl}-3H-quinazolin-4-one;
7-[4-amino-4-(4-chloro-phenyl)-piperidin-1-yl]-3H-quinazolin-4-one;
7-[4-aminomethyl-4-(4-chloro-benzyl)-piperidin-1-yl]-3H-quinazolin-4-one; and
7-[4-Aminomethyl-4-(4-chloro-benzyl)-piperidin-1-yl]-1-methyl-1H-quinazoline-2,4-dione;
or salts, solvates, tautomers or N-oxides thereof.

104. A compound according to claim 81 in the form of a salt or N-oxide.

105. A method of modulating a cellular process by inhibiting the activity of protein kinase A and/or protein kinase B using a compound of the formula set forth in claim 79.

106. A pharmaceutical composition comprising a compound of the formula set forth in claim 81 and a pharmaceutically acceptable carrier.

107. A method for the diagnosis and treatment of a disease state or condition mediated by protein kinase A and/or protein kinase B, which method comprises (i) screening a patient to determine whether a disease or condition from which the patient is or may be suffering is one which would be susceptible to treatment with a compound having activity against protein kinase A and/or protein kinase B; and (ii) where it is indicated that the disease or condition from which the patient is thus susceptible, thereafter administering to the patient a compound of the formula set forth in claim 79.

108. A process for the preparation of a compound of the formula set forth in claim 81, which process comprises:
(a) when E is CONR⁷, the reaction of a compound of the formula (X) with a compound of the formula (X¹) or an activated derivative thereof, under amide forming conditions: 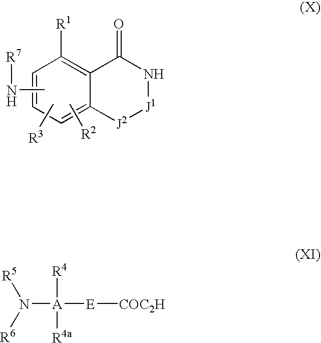
(b) when E is NR⁷CO, the reaction of a compound of the formula (XII) or an activated derivative thereof with a compound of the formula (XIII) under amide forming conditions: 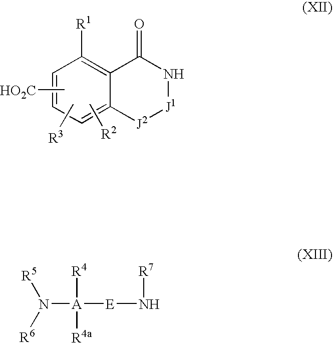
(c) when E is O or S, the reaction of a compound of the formula (XIV) or an N-protected form thereof with a compound of the formula (XV): 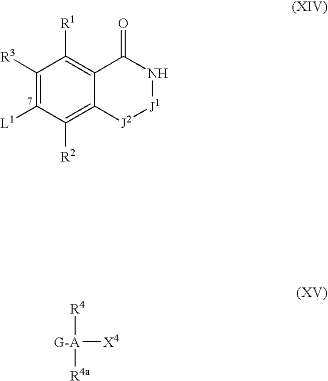
where L¹ is a leaving group or atom such as fluorine and X⁴ is OH or SH or an anion thereof in the presence of a base;
(d) when E is O or S, the reaction of a compound of the formula (XIVa) or an N-protected form thereof with a compound of the formula (XVa): 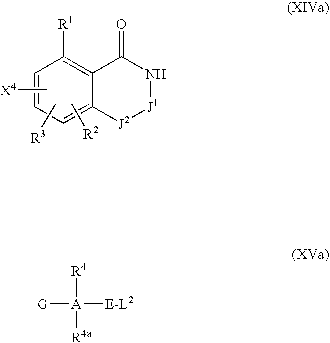
where L² is a leaving group or atom such as bromine and X⁴ is OH or SH or an anion thereof, in the presence of a base;
(e) when E is NR⁷, the reaction of a compound of the formula (XIV) with a compound of the formula (XIII), wherein (XIII) and (XIV) are as hereinbefore defined;
(f) when E is CONR⁷, A is a bond, R⁴ and R^{4a} are absent and R⁵ is hydrogen, the reaction of a compound of the formula (X) with a compound of the formula R⁶NCO under urea forming conditions;
(g) when E is CR⁸R^{8a}, the coupling of a compound of the formula (XVI) where A′ is the residue of the group A and R^{x} is hydrogen, methyl or ethyl, with a compound of the formula (XVIIa) or (XVIIb) where Hal is a halogen such as bromine, in the presence of a transition metal catalyst such as a palladium catalyst and/or a copper catalyst: 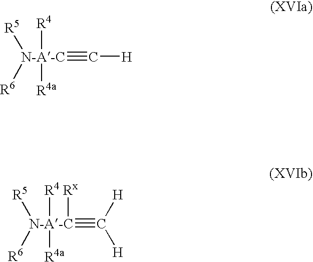 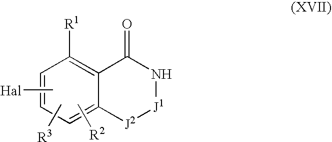
(h) when E is O, S or NR⁷, the reaction of a compound of the formula (XVII) or an N-protected derivative thereof, with a compound of the formula (XIII) or (XV) in the presence of a palladium or copper catalyst; and
(i) optionally the conversion of one compound of the formula (I) to another compound of the formula (I).
